(19) United States
(12) Patent Application Publication
(10) Pub. No.: US 2024/0252036 A1
(43) Pub. Date: Aug. 1, 2024

(54) VISION-BASED COGNITIVE IMPAIRMENT TESTING DEVICE, SYSTEM AND METHOD

(71) Applicant: EVOLUTION OPTIKS LIMITED, Christ Church (BB)

(72) Inventors: Guillaume LUSSIER, Montreal (CA); Faleh Mohammad Faleh ALTAL, Montreal (CA); Khaled EL-MONAJJED, Laval (CA); Yaiza GARCIA, Montreal (CA); Soumya Ramesh KUNDER, Montreal (CA)

(21) Appl. No.: 18/548,857

(22) PCT Filed: Mar. 3, 2022
(86) PCT No.: PCT/US2022/018791

§ 371 Date: Sep. 1, 2023

Related U.S. Application Data
(60) Provisional application No. 63/200,433, filed on Mar. 5, 2021.
(60) Provisional application No. 63/179,021, filed on Apr. 23, 2021.
(60) Provisional application No. 63/179,057, filed on Apr. 23, 2021.

(51) Int. Cl. A61B 3/032; G02B 30/10

(52) U.S. Cl. CPC A61B 3/032; G02B 30/10

(57) ABSTRACT

Described are various embodiments of a vision-based testing device for digitally implementing a vision-based test for a user using both their left and right eye simultaneously. In one embodiment, the device comprises: left and right display portions comprising respective pixel arrays; corresponding light field shaping element (LFSE) arrays of light field shaping elements respectively disposed at a distance from said display portions so to at least partially govern respective left and right light fields projected on the user's left and right eye, respectively; and a digital data processor operable on pixel data for designated visual digital test content, to simultaneously render said designated visual digital test content in accordance with the vision-based test to be simultaneously perceived by the left and right eye, respectively, to be at a common virtual position so to invoke a natural binocular eye vergence response corresponding to said common virtual position.

### FIELD OF THE DISCLOSURE

The present disclosure relates to cognitive impairment testing devices, and in particular, to a vision-based cognitive impairment testing device, system and method.

### BACKGROUND

The Centers for Disease Control estimates that more than 1.6 million people in the United States suffer a concussion—or traumatic brain injury—every year. It was once assumed that the hallmark of a concussion was a loss of consciousness. More recent evidence, however, does not support that. The majority of people diagnosed with a concussion do not experience any loss of consciousness. The most common immediate symptoms are amnesia and confusion. Since the visual system of a person is a relatively easily accessible part of the nervous system, it may be used to evaluate possible brain injury resulting from a concussion or similar. Indeed, the visual system involves half of the brain circuits and many of them are vulnerable to head injury. Traditionally, vision has not been properly used as a diagnostic tool, but a more careful analysis could provide a powerful tool to save precious time in the diagnosis and early treatment. For example, post-concussion syndrome (PCS) involves a constellation of symptoms and/or signs that commonly follow traumatic brain injury (TBI). After a concussion, the oculomotor control, or eye movement, may be disrupted. Examining the oculomotor system may thus provide valuable information in evaluating the presence or degree of cognitive impairment, for example caused by a concussion or similar.

For example, after mild TBI (concussion), common visual disorders that may ensue include convergence insufficiency (CI), accommodative insufficiency (AI), and mild saccadic dysfunction (SD). Since a mild concussion is frequently associated with abnormalities of saccades, pursuit eye movements, convergence, accommodation, and the vestibular-ocular reflex, testing or evaluating the vision system or eyes of an individual suspected of being cognitively impaired may be used to detect abnormalities in some of these aspects.

This background information is provided to reveal information believed by the applicant to be of possible relevance. No admission is necessarily intended, nor should be construed, that any of the preceding information constitutes prior art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of the embodiments of the disclosure or to delineate their scope beyond that which is explicitly or implicitly described by the following description and claims.

A need exists for a vision-based cognitive impairment testing device, system and method, that overcome some of the drawbacks of known techniques, or at least, provide a useful alternative thereto. Some aspects of disclosure provide embodiments of such systems, methods, and devices.

In accordance with one aspect, there is provided a vision-based testing device for digitally implementing a vision-based test for a user using both their left and right eye simultaneously, the device comprising: left and right display portions comprising respective pixel arrays; corresponding light field shaping element (LFSE) arrays of light field shaping elements respectively disposed at a distance from said display portions so to at least partially govern respective left and right light fields projected on the user's left and right eye, respectively, wherein perception of said respective left and right light fields is at least partially constrained to the left and right eye, respectively; and a digital data processor operable on pixel data for designated visual digital test content, to simultaneously render said designated visual digital test content via said respective pixel arrays in accordance with the vision-based test to be respectively projected toward respective user pupil locations in accordance with respective light field view zones generated via said respective pixel arrays and corresponding LFSE arrays to be simultaneously perceived by the left and right eye, respectively, to be at a common virtual position relative to the left and right eye so to invoke a natural binocular eye vergence response corresponding to said common virtual position.

In one embodiment, the common virtual position comprises a virtual depth position relative to said display portions.

In one embodiment, said left and right display portions comprise respective displays, and wherein said corresponding LFSE arrays comprise respective microlens arrays.

In one embodiment, said perception of said respective left and right light fields is at least partially constrained to the left and right eye via a physical barrier.

In one embodiment, said LFSE arrays comprise a microlens array.

In one embodiment, said common virtual position is a variable three-dimensional (3D) position that varies during execution of the vision-based test to dynamically adjust a perceived depth location of said designated visual digital test content and thereby invoke a variable binocular eye vergence response thereto.

In one embodiment, the vision-based test comprises a vergence test.

In one embodiment, said common virtual position is a variable two-dimensional (2D) location on a plane parallel to said display portions that varies during execution of the test to dynamically adjust a common perceived lateral location of said designated visual digital test content.

In one embodiment, the vision-based test comprises at least one of a saccades test or a smooth pursuit test.

In one embodiment, said designated visual digital test content comprises at least one of an optotype, a symbol, an image, a spot or a flash.

In one embodiment, said digital data processor is operable to adjust rendering of said designated visual digital test content via said corresponding LFSE arrays so to accommodate for a visual aberration in at least one of the left or right eye.

In one embodiment, said visual aberration comprises distinct respective visual aberrations for the left and right eye.

In one embodiment, the device further comprises a pupil or eye tracking interface for tracking a motion of the left and right eye during execution of the vision-based test.

In one embodiment, said digital data processor is operable on said pixel data for each of the left and right display portions, respectively, to digitally: project a given ray trace between each given pixel and a given pupil location given a direction of a light field emanated by said given pixel based on a given LFSE intersected thereby, to intersect said designated visual digital test content at said common virtual position or at its respective corresponding retinal image projections thereof; and for each said given pixel, associate a given adjusted image pixel value designated as a function of said intersection.

In one embodiment, the vision-based testing device further comprises a selectable or tunable lens to extend a dynamic range of said perceived depth location.

In one embodiment, the vision-based testing device further comprises respective selectable or tunable lenses tunable to dynamically optically force the left and right eye to accommodate such that said designated visual digital test content is simultaneously perceived by the left and right eye, respectively, to be at said common virtual position relative.

In one embodiment, the digital data processor is further operable on pixel data for said designated visual digital test content to further adjust perception thereof in dynamically optically forcing the left and right eye to accommodate such that said designated visual digital test content is simultaneously perceived by the left and right eye, respectively, to be at said common virtual position.

In one embodiment, the digital data processor is further operable on pixel data for said designated visual digital test content to accommodate for a reduced user visual acuity such that said designated visual digital test content is simultaneously perceived by the left and right eye, respectively, to be at said common virtual position relative to the left and right eye without an intervening corrective lens adapted for said reduced visual acuity.

In one embodiment, the reduced user visual acuity comprises distinct respective reduced visual acuities for each of the right and left eye.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a diagrammatical view of an electronic device having a digital display, in accordance with one embodiment;

FIGS. 2A and 2B are exploded and side views, respectively, of an assembly of a light field display for an electronic device, in accordance with one embodiment;

FIGS. 3A, 3B and 3C schematically illustrate normal vision, blurred vision, and corrected vision in accordance with one embodiment, respectively;

FIG. 4 is a schematic diagram of a single light field pixel defined by a convex lenslet or microlens overlaying an underlying pixel array and disposed at or near its focus to produce a substantially collimated beam, in accordance with one embodiment;

FIG. 5 is another schematic exploded view of an assembly of a light field display in which respective pixel subsets are aligned to emit light through a corresponding microlens or lenslet, in accordance with one embodiment;

FIG. 6 is an exemplary diagram of a light field pattern that, when properly projected by a light field display, produces a corrected image exhibiting reduced blurring for a viewer having reduced visual acuity, in accordance with one embodiment;

FIGS. 7A and 7B are photographs of a Snellen chart, as illustratively viewed by a viewer with reduced acuity without image correction (blurry image in FIG. 7A) and with image correction via a light field display (corrected image in FIG. 7B), in accordance with one embodiment;

FIG. 8 is a schematic diagram of a portion of a hexagonal lenslet array disposed at an angle relative to an underlying pixel array, in accordance with one embodiment;

FIGS. 9A and 9B are photographs as illustratively viewed by a viewer with reduced visual acuity without image correction (blurry image in FIG. 9A) and with image correction via a light field display having an angularly mismatched lenslet array (corrected image in FIG. 9B), in accordance with one embodiment;

FIGS. 10A and 10B are photographs as illustratively viewed by a viewer with reduced visual acuity without image correction (blurry image in FIG. 10A) and with image correction via a light field display having an angularly mismatched lenslet array (corrected image in FIG. 10B), in accordance with one embodiment;

FIG. 11 is a process flow diagram of an illustrative ray-tracing rendering process, in accordance with one embodiment;

FIGS. 12 and 13 are process flow diagrams of exemplary input constant parameters and variables, respectively, for the ray-tracing rendering process of FIG. 11, in accordance with one embodiment;

FIGS. 14A to 14C are schematic diagrams illustrating certain process steps of FIG. 11;

FIG. 15 is a process flow diagram of an exemplary process for computing the center position of an associated light field shaping unit in the ray-tracing rendering process of FIG. 11, in accordance with one embodiment;

FIGS. 16A and 16B are schematic diagrams illustrating an exemplary hexagonal light field shaping layer with a corresponding hexagonal tile array, in accordance with one embodiment;

FIGS. 17A and 17B are schematic diagrams illustrating overlaying a staggered rectangular tile array over the hexagonal tile array of FIGS. 16A and 16B, in accordance with one embodiment;

FIGS. 18A to 18C are schematic diagrams illustrating the associated regions of neighboring hexagonal tiles within a single rectangular tile, in accordance with one embodiment;

FIG. 19 is process flow diagram of an illustrative ray-tracing rendering process, in accordance with another embodiment;

FIGS. 20A to 20D are schematic diagrams illustrating certain process steps of FIG. 19;

FIGS. 21A and 21B are schematic diagrams illustrating pixel and subpixel rendering, respectively, in accordance with some embodiments;

FIGS. 22A and 22B are schematic diagrams of an LCD pixel array defined by respective red (R), green (G) and blue (B) subpixels, and rendering an angular image edge using pixel and subpixel rendering, respectively, in accordance with one embodiment;

FIG. 23 is a schematic diagram of one of the pixels of FIG. 22A, showing measures for independently accounting for subpixels thereof apply subpixel rendering to the display of a corrected image through a light field display, in accordance with one embodiment; and

FIG. 24 is a process flow diagram of an illustrative ray-tracing rendering process for rendering a light field originating from multiple distinct virtual image planes, in accordance with one embodiment;

FIG. 25 is a process flow diagram of an exemplary process for iterating over multiple virtual image planes in the ray-tracing rendering process of FIG. 24, in accordance with one embodiment;

FIGS. 26A to 26D are schematic diagrams illustrating certain process steps of FIG. 25;

FIG. 27 is a process flow diagram of an illustrative ray-tracing rendering process for rendering a light field originating from multiple distinct image planes, in accordance with one embodiment;

FIG. 28 is a process flow diagram of an exemplary process for iterating over multiple image planes in the ray-tracing rendering process of FIG. 27, in accordance with one embodiment;

FIGS. 29A and 29B are schematic diagrams illustrating an example of a subjective visual acuity test using the ray-tracing rendering process of FIG. 25 or FIG. 27, in accordance with one embodiment;

FIG. 30 is a schematic diagram of an exemplary vision testing system, in accordance with one embodiment;

FIGS. 31A to 31C are schematic diagrams of exemplary light field refractors/phoropters, in accordance with different embodiments;

FIG. 32 is a plot of the angular resolution of an exemplary light field display as a function of the dioptric power generated, in accordance with one embodiment;

FIGS. 33A to 33D are schematic plots of the image quality generated by a light field refractor/phoropter as a function of the dioptric power generated by using in combination with the light field display (A) no refractive component, (B) one refractive component, (C) and (D) a multiplicity of refractive components;

FIGS. 34A and 34B are perspective internal views of exemplary light field refractors/phoropters showing a casing thereof in cross-section, in accordance with one embodiment;

FIG. 35 is a perspective view of an exemplary light field refractor/phoropter combining side-by-side two of the units shown in FIGS. 34A and 34B for evaluating both eyes at the same time, in accordance with one embodiment;

FIG. 36 is a process flow diagram of an exemplary dynamic subjective vision testing method, in accordance with one embodiment;

FIG. 37 is a schematic diagram of an exemplary light field image showing two columns of optotypes at different dioptric power for the method of FIG. 36, in accordance with one embodiment;

FIG. 38 is a schematic diagram of an exemplary light field refractor/phoropter adapted for cognitive impairment detection, according to one embodiment;

FIG. 39 is a flow process diagram of an exemplary cognitive impairment detection method, according to one embodiment;

FIG. 40 is a diagram illustrating an exemplary process flow for generating gaze tracking output using a cognitive impairment assessment system, in accordance with various embodiments;

FIG. 41 is a schematic diagram illustrating various parameters used in gaze tracking analysis, in accordance with various embodiments;

FIG. 42 is a schematic diagram illustrating an exemplary oculomotor test that may be performed using a cognitive impairment assessment system, in accordance with one embodiment;

FIG. 43 is an illustrative plot of processed saccade data, in accordance with one embodiment;

FIG. 44 is an illustrative plot of processed gaze tracking data, in accordance with one embodiment;

FIG. 45 is a schematic illustrating an exemplary metric assessed using a cognitive impairment assessment system, in accordance with one embodiment;

FIG. 46 is an illustrative plot of accommodation data acquired using a cognitive impairment assessment system, in accordance with one embodiment;

FIGS. 47 to 49 are schematics illustrating exemplary oculomotor tests that may be performed using a cognitive impairment assessment system, in accordance with various embodiments;

FIG. 50A is a schematic illustrating an exemplary oculomotor test that may be performed using a cognitive impairment assessment device, and FIG. 50B is an illustrative plot of exemplary gaze tracking data acquired during the oculomotor test of FIG. 16A, in accordance with one embodiment;

FIG. 51A is a schematic illustrating an exemplary oculomotor test that may be performed using a cognitive impairment assessment device, and FIG. 51B is an illustrative plot of exemplary gaze tracking data acquired during the oculomotor test of FIG. 51A, in accordance with one embodiment;

FIG. 52A is a schematic illustrating an exemplary oculomotor test that may be performed using a cognitive impairment assessment device, and FIG. 52B is an illustrative plot of exemplary gaze tracking data acquired during the oculomotor test of FIG. 52A, in accordance with one embodiment;

FIG. 53 is a schematic of an exemplary oculomotor test that may be performed using a cognitive impairment assessment device, in accordance with one embodiment;

FIGS. 54A and 54B are schematics of an exemplary oculomotor test that may be performed using a cognitive impairment assessment device, in accordance with one embodiment;

FIGS. 55A and 55B are schematics of an exemplary oculomotor test that may be performed using a cognitive impairment assessment device, in accordance with one embodiment;

FIGS. 56A and 56B are schematics illustrating exemplary oculomotor tests that may be performed using a cognitive impairment assessment device, and FIG. 56C is an exemplary plot of OKN versus time, in accordance with various embodiments;

FIG. 57 is an exemplary plot of eye movement versus time acquired during a cognitive impairment assessment system, in accordance with one embodiment;

FIGS. 58A and 58B are flow diagrams illustrated additional steps used to enable stereoscopic vision for methods 1100 and 2400 (58A) or methods 1900 and 2700 (58B), in accordance with one embodiment;

FIGS. 59 to 61 are schematic diagrams illustrating various implementations of light field rendering for a binocular device, in accordance with multiple embodiments.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

The systems and methods described herein provide, in accordance with different embodiments, different examples of a light field vision testing device, such as a light field refractor and/or refractor, and an adjusted pixel rendering method therefor, which can be further or alternatively used as a cognitive impairment testing device or system. For example, different vision or visual system testing tools may rely on the herein described solutions to provide a fast and reliable response when a head injury happens. For example, such tools may be highly beneficial, in some embodiments or applications, for a quick evaluation, assessment or screening (e.g. in a clinical environment, in the field and/or through other direct/remote configurations), especially when it may differentiate between mild and no concussion. Most people with visual complaints after a concussion have 20/20 distance visual acuity so more specific testing of near acuity, convergence amplitudes, ocular motility, and peripheral vision can be done.

The light field rendering and vision testing tools described below may be used to implement the required tests to evaluate some of the signs and symptoms of TBI. These and other such applications will be described in further detail below.

As noted above, the devices, displays and methods described herein may allow a user's perception of one or more input images (or input image portions), where each image or image portion is virtually located at a distinct image plane/depth location, to be adjusted or altered using the light field display. These may be used, as described below, to provide vision correction for a user viewing digital displays, but the same light field displays and rendering technology, as detailed below and according to different embodiments, may equally be used or be implemented in a refractor or phoropter-like device to test, screen, diagnose and/or deduce a patient's reduced visual acuity, or again, to conduct one or more vision-based cognitive impairment tests.

In accordance with some embodiments, different vision testing devices and systems as described herein may be contemplated so to replace or complement traditional vision testing devices such as refractors and/or phoropters, in which traditional devices different optotypes are shown to a user in sequence via changing and/or compounding optical elements (lenses, prisms, etc.) so to identify an optical combination that best improves the user's perception of these displayed optotypes. As will be described in greater detail below, embodiments as described herein introduce lightfield display technologies and image rendering techniques, alone or in combination with complementary optical elements such as refractive lens, prisms, etc., to provide, amongst other benefits, for greater vision testing versatility, compactness, portability, range, precision, and/or other benefits as will be readily appreciated by the skilled artisan. Accordingly, while the terms lightfield refractor or phoropters will be used interchangeably herein to reference the implementation of different embodiments of a more generally defined lightfield vision testing device and system, the person of ordinary skill in the art will appreciate the versatility of the herein described implementation of light field rendering techniques, and ray tracing approaches detailed herein with respect to some embodiments, in the provision of effective lightfield vision and/or cognitive impairment testing devices and systems in general.

As noted above, some of the herein described embodiments provide for digital display devices, or devices encompassing such displays, for use by users having reduced visual acuity, whereby images ultimately rendered by such devices can be dynamically processed to accommodate the user's reduced visual acuity so that they may consume rendered images without the use of corrective eyewear, as would otherwise be required. Accordingly, such embodiments can be dynamically controlled to progressively adjust a user's perception of rendered images or image portions (e.g. optotype within the context of a blur test for example) until an optimized correction is applied that optimizes the user's perception. Perception adjustment parameters used to achieve this optimized perception can then be translated into a proposed vision correction prescription to be applied to corrective eyewear. Conversely, a user's vision correction eyewear prescription can be used as input to dictate selection of applied vision correction parameters and related image perception adjustment, to validate or possibly further fine tune the user's prescription, for example, and progressively adjusting such correction parameters to test for the possibility of a further improvement. As noted above, embodiments are not to be limited as such as the notions and solutions described herein may also be applied to other technologies in which a user's perception of an input image to be displayed can be altered or adjusted via the light field display. However, for the sake of illustration, a number of the herein described embodiments will be described as allowing for implementation of digitally adaptive vision tests such that individuals with such reduced visual acuity can be exposed to distinct perceptively adjusted versions of an input image(s) (e.g. optotypes) to subjectively ascertain a potentially required or preferred vision correction.

In accordance with various embodiments, a light field-based cognitive assessment may take advantage of the presentation of content to the subject in accordance with a perception adjustment designated so to accommodate a reduced visual acuity of the subject as mentioned above. That is, a conventional cognitive assessment targeting the oculomotor system may comprise presenting content (e.g. a test for assessing saccadic movement, smooth pursuit, etc.) at a fixed distance from the subject's eye(s) (e.g. from a 2D tablet screen or computer monitor), requiring a subject having a reduced visual acuity (e.g. farsighted, nearsighted, or the like) to wear prescriptive lenses to properly view the content. Conversely, various embodiments herein described relate to the operation of a light field assessment system for the presentation of content having a dioptric correction or optotype applied thereto (e.g. +3.0 D, −4.25 D, etc.). Accordingly, various embodiments allow the subject to properly view content without glasses or another form of corrective lenses, which would otherwise hinder the assessment by, for instance, interfering with eye tracking, inhibiting proper alignment of the device on the subject's face, or the like. Such content adjustments may be presented in addition to, for instance, dioptric corrections or image depth plane adjustments inherent in, for instance, a near point of accommodation or vergence assessment.

Similarly, and in accordance with various embodiments, a cognitive assessment system may be operable to render content in accordance with different dioptric corrections in different viewing regions (e.g. different screens) corresponding to respective eyes of the subject. For example, if a subject has eyes of differing visual acuity (e.g. prescriptions of +1.25 D for the right eye and +2.5 for the left eye), different dioptric shifts or perception adjustments may be rendered by the respective screen(s) corresponding to each eye of the subject. In accordance with different embodiments, such respective perception adjustments for each eye may be applied for either monocular or binocular assessments.

It will further be appreciated that while the application of such dioptric corrections may improve a quality or outcome of cognitive assessment tests, the dioptric correction required for a subject to clearly see displayed content may itself constitute a diagnostic test, in accordance with one embodiment. For example, a cognitive impairment assessment device may be operable to assess the visual acuity of a user through, for instance, the display of different optotypes. If a subject is observed to not exhibit a prior baseline of visual acuity, they may be exhibiting signs of a cognitive impairment.

Generally, digital displays as considered herein will comprise a set of image rendering pixels and a corresponding set of light field shaping elements that at least partially govern a light field emanated thereby to produce a perceptively adjusted version of the input image, notably distinct perceptively adjusted portions of an input image or input scene, which may include distinct portions of a same image, a same 2.5D/3D scene, or distinct images (portions) associated with different image depths, effects and/or locations and assembled into a combined visual input. For simplicity, the following will generally consider distinctly addressed portions or segments as distinct portions of an input image, whether that input image comprises a singular image having distinctly characterized portions, a digital assembly of distinctly characterized images, overlays, backgrounds, foregrounds or the like, or any other such digital image combinations.

In some examples, light field shaping elements may take the form of a light field shaping layer or like array of optical elements to be disposed relative to the display pixels in at least partially governing the emanated light field. As described in further detail below, such light field shaping layer elements may take the form of a microlens and/or pinhole array, or other like arrays of optical elements, or again take the form of an underlying light shaping layer, such as an underlying array of optical gratings or like optical elements operable to produce a directional pixelated output.

Within the context of a light field shaping layer, as described in further detail below in accordance with some embodiments, the light field shaping layer can be disposed at a pre-set distance from the pixelated display so to controllably shape or influence a light field emanating therefrom. For instance, each light field shaping layer can be defined by an array of optical elements centered over a corresponding subset of the display's pixel array to optically influence a light field emanating therefrom and thereby govern a projection thereof from the display medium toward the user, for instance, providing some control over how each pixel or pixel group will be viewed by the viewer's eye(s). As will be further detailed below, arrayed optical elements may include, but are not limited to, lenslets, microlenses or other such diffractive optical elements that together form, for example, a lenslet array; pinholes or like apertures or windows that together form, for example, a parallax or like barrier; concentrically patterned barriers, e.g. cut outs and/or windows, such as a to define a Fresnel zone plate or optical sieve, for example, and that together form a diffractive optical barrier (as described, for example, in Applicant's co-pending U.S. application Ser. No. 15/910,908, the entire contents of which are hereby incorporated herein by reference); and/or a combination thereof, such as for example, a lenslet array whose respective lenses or lenslets are partially shadowed or barriered around a periphery thereof so to combine the refractive properties of the lenslet with some of the advantages provided by a pinhole barrier.

In operation, the display device will also generally invoke a hardware processor operable on image pixel (or subpixel) data for an image to be displayed to output corrected or adjusted image pixel data to be rendered as a function of a stored characteristic of the light field shaping elements and/or layer (e.g. layer distance from display screen, distance between optical elements (pitch), absolute relative location of each pixel or subpixel to a corresponding optical element, properties of the optical elements (size, diffractive and/or refractive properties, etc.), or other such properties, and a selected vision correction or adjustment parameter related to the user's reduced visual acuity or intended viewing experience. While light field display characteristics will generally remain static for a given implementation (i.e. a given shaping element and/or layer will be used and set for each device irrespective of the user), image processing can, in some embodiments, be dynamically adjusted as a function of the user's visual acuity or intended application so to actively adjust a distance of a virtual image plane, or perceived image on the user's retinal plane given a quantified user eye focus or like optical aberration(s), induced upon rendering the corrected/adjusted image pixel data via the static optical layer and/or elements, for example, or otherwise actively adjust image processing parameters as may be considered, for example, when implementing a viewer-adaptive pre-filtering algorithm or like approach (e.g. compressive light field optimization), so to at least in part govern an image perceived by the user's eye(s) given pixel or subpixel-specific light visible thereby through the layer.

Accordingly, a given device may be adapted to compensate for different visual acuity levels and thus accommodate different users and/or uses. For instance, a particular device may be configured to implement and/or render an interactive graphical user interface (GUI) that incorporates a dynamic vision correction scaling function that dynamically adjusts one or more designated vision correction parameter(s) in real-time in response to a designated user interaction therewith via the GUI. For example, a dynamic vision correction scaling function may comprise a graphically rendered scaling function controlled by a (continuous or discrete) user slide motion or like operation, whereby the GUI can be configured to capture and translate a user's given slide motion operation to a corresponding adjustment to the designated vision correction parameter(s) scalable with a degree of the user's given slide motion operation. These and other examples are described in Applicant's co-pending U.S. patent application Ser. No. 15/246,255, the entire contents of which are hereby incorporated herein by reference.

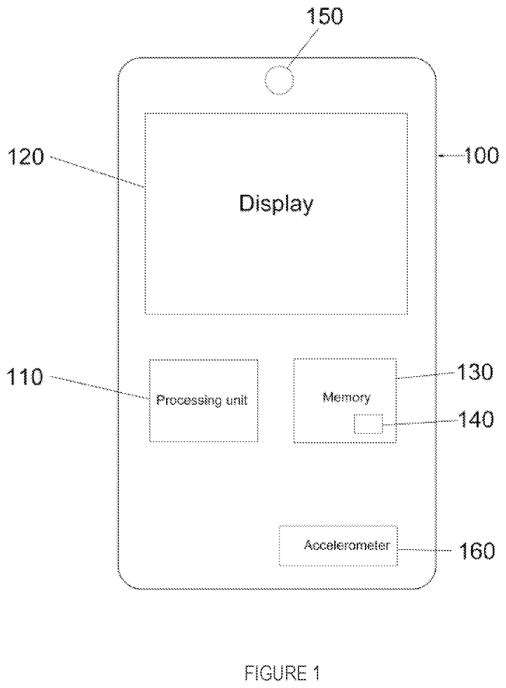

With reference to FIG. 1, and in accordance with one embodiment, a digital display device, generally referred to using the numeral 100, will now be described. In this example, the device 100 is generally depicted as a smartphone or the like, though other devices encompassing a graphical display may equally be considered, such as tablets, e-readers, watches, televisions, GPS devices, laptops, desktop computer monitors, televisions, smart televisions, handheld video game consoles and controllers, vehicular dashboard and/or entertainment displays, and the like.

In the illustrated embodiment, the device 100 comprises a processing unit 110, a digital display 120, and internal memory 130. Display 120 can be an LCD screen, a monitor, a plasma display panel, an LED or OLED screen, or any other type of digital display defined by a set of pixels for rendering a pixelated image or other like media or information. Internal memory 130 can be any form of electronic storage, including a disk drive, optical drive, read-only memory, random-access memory, or flash memory, to name a few examples. For illustrative purposes, memory 130 has stored in it vision correction application 140, though various methods and techniques may be implemented to provide computer-readable code and instructions for execution by the processing unit in order to process pixel data for an image to be rendered in producing corrected pixel data amenable to producing a corrected image accommodating the user's reduced visual acuity (e.g. stored and executable image correction application, tool, utility or engine, etc.). Other components of the electronic device 100 may optionally include, but are not limited to, one or more rear and/or front-facing camera(s) 150, an accelerometer 160 and/or other device positioning/orientation devices capable of determining the tilt and/or orientation of electronic device 100, and the like.

For example, the electronic device 100, or related environment (e.g. within the context of a desktop workstation, vehicular console/dashboard, gaming or e-learning station, multimedia display room, etc.) may include further hardware, firmware and/or software components and/or modules to deliver complementary and/or cooperative features, functions and/or services. For example, in some embodiment, and as will be described in greater detail below, a pupil/eye tracking system may be integrally or cooperatively implemented to improve or enhance corrective image rending by tracking a location of the user's eye(s)/pupil(s) (e.g. both or one, e.g. dominant, eye(s)) and adjusting light field corrections accordingly. For instance, the device 100 may include, integrated therein or interfacing therewith, one or more eye/pupil tracking light sources, such as one or more infrared (IR) or near-IR (NIR) light source(s) to accommodate operation in limited ambient light conditions, leverage retinal retro-reflections, invoke corneal reflection, and/or other such considerations. For instance, different IR/NIR pupil tracking techniques may employ one or more (e.g. arrayed) directed or broad illumination light sources to stimulate retinal retro-reflection and/or corneal reflection in identifying a tracking a pupil location. Other techniques may employ ambient or IR/NIR light-based machine vision and facial recognition techniques to otherwise locate and track the user's eye(s)/pupil(s). To do so, one or more corresponding (e.g. visible, IR/NIR) cameras may be deployed to capture eye/pupil tracking signals that can be processed, using various image/sensor data processing techniques, to map a 3D location of the user's eye(s)/pupil(s). In the context of a mobile device, such as a mobile phone, such eye/pupil tracking hardware/software may be integral to the device, for instance, operating in concert with integrated components such as one or more front facing camera(s), onboard IR/NIR light source(s) and the like. In other user environments, such as in a vehicular environment, eye/pupil tracking hardware may be further distributed within the environment, such as dash, console, ceiling, windshield, mirror or similarly-mounted camera(s), light sources, etc.

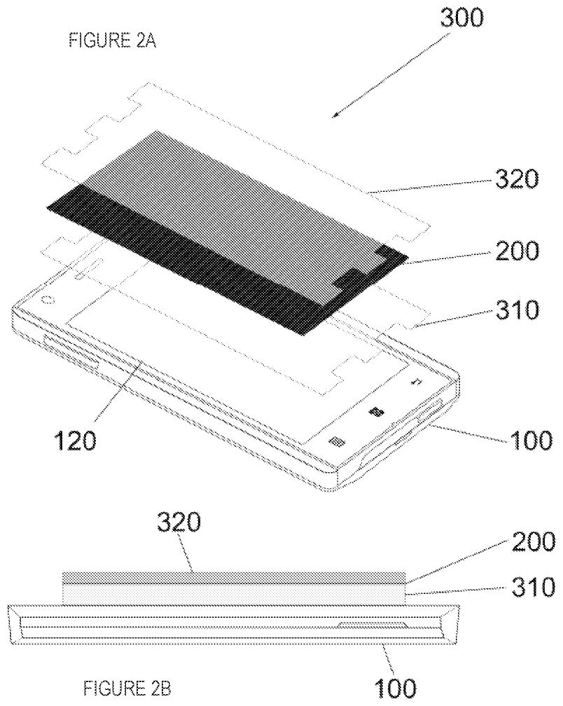

With reference to FIGS. 2A and 2B, the electronic device 100, such as that illustrated in FIG. 1, is further shown to include a light field shaping layer (LFSL) 200 overlaid atop a display 120 thereof and spaced therefrom via a transparent spacer 310 or other such means as may be readily apparent to the skilled artisan. An optional transparent screen protector 320 is also included atop the layer 200.

For the sake of illustration, the following embodiments will be described within the context of a light field shaping layer defined, at least in part, by a lenslet array comprising an array of microlenses (also interchangeably referred to herein as lenslets) that are each disposed at a distance from a corresponding subset of image rendering pixels in an underlying digital display. It will be appreciated that while a light field shaping layer may be manufactured and disposed as a digital screen overlay, other integrated concepts may also be considered, for example, where light field shaping elements are integrally formed or manufactured within a digital screen's integral components such as a textured or masked glass plate, beam-shaping light sources (e.g. directional light sources and/or backlit integrated optical grating array) or like component.

Accordingly, each lenslet will predictively shape light emanating from these pixel subsets to at least partially govern light rays being projected toward the user by the display device. As noted above, other light field shaping layers may also be considered herein without departing from the general scope and nature of the present disclosure, whereby light field shaping will be understood by the person of ordinary skill in the art to reference measures by which light, that would otherwise emanate indiscriminately (i.e. isotropically) from each pixel group, is deliberately controlled to define predictable light rays that can be traced between the user and the device's pixels through the shaping layer.

For greater clarity, a light field is generally defined as a vector function that describes the amount of light flowing in every direction through every point in space. In other words, anything that produces or reflects light has an associated light field. The embodiments described herein produce light fields from an object that are not “natural” vector functions one would expect to observe from that object. This gives it the ability to emulate the “natural” light fields of objects that do not physically exist, such as a virtual display located far behind the light field display, which will be referred to now as the ‘virtual image’. As noted in the examples below, in some embodiments, light field rendering may be adjusted to effectively generate a virtual image on a virtual image plane that is set at a designated distance from an input user pupil location, for example, so to effectively push back, or move forward, a perceived image relative to the display device in accommodating a user's reduced visual acuity (e.g. minimum or maximum viewing distance). In yet other embodiments, light field rendering may rather or alternatively seck to map the input image on a retinal plane of the user, taking into account visual aberrations, so to adaptively adjust rendering of the input image on the display device to produce the mapped effect. Namely, where the unadjusted input image would otherwise typically come into focus in front of or behind the retinal plane (and/or be subject to other optical aberrations), this approach allows to map the intended image on the retinal plane and work therefrom to address designated optical aberrations accordingly. Using this approach, the device may further computationally interpret and compute virtual image distances tending toward infinity, for example, for extreme cases of presbyopia. This approach may also more readily allow, as will be appreciated by the below description, for adaptability to other visual aberrations that may not be as readily modeled using a virtual image and image plane implementation. In both of these examples, and like embodiments, the input image is digitally mapped to an adjusted image plane (e.g. virtual image plane or retinal plane) designated to provide the user with a designated image perception adjustment that at least partially addresses designated visual aberrations. Naturally, while visual aberrations may be addressed using these approaches, other visual effects may also be implemented using similar techniques.

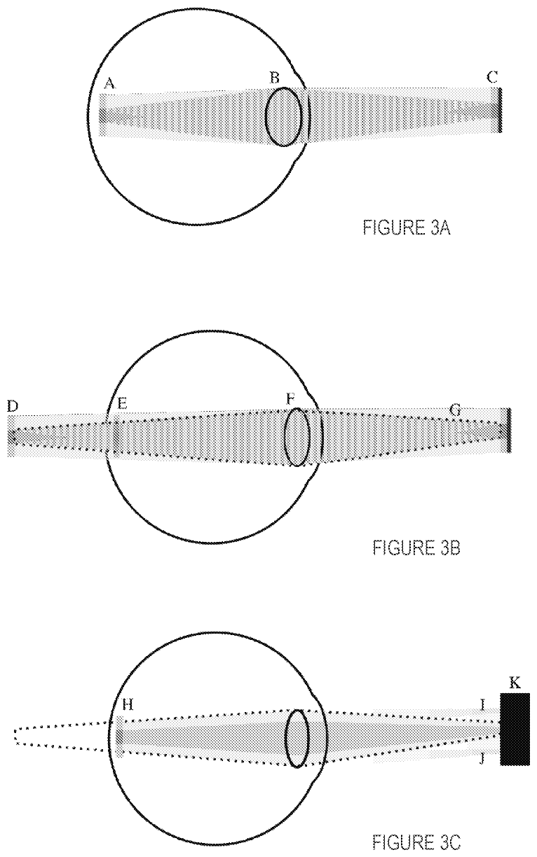

In one example, to apply this technology to vision correction, consider first the normal ability of the lens in an eye, as schematically illustrated in FIG. 3A, where, for normal vision, the image is to the right of the eye (C) and is projected through the lens (B) to the retina at the back of the eye (A). As comparatively shown in FIG. 3B, the poor lens shape and inability to accommodate (F) in presbyopia causes the image to be focused past the retina (D) forming a blurry image on the retina (E). The dotted lines outline the path of a beam of light (G). Naturally, other optical aberrations present in the eye will have different impacts on image formation on the retina. To address these aberrations, a light field display (K), in accordance with some embodiments, projects the correct sharp image (H) on the retina for an eye with a crystalline lens which otherwise could not accommodate sufficiently to produce a sharp image. The other two light field pixels (I) and (J) are drawn lightly, but would otherwise fill out the rest of the image.

As will be appreciated by the skilled artisan, a light field as seen in FIG. 3C cannot be produced with a ‘normal’ two-dimensional display because the pixels' light field emits light isotropically. Instead it is necessary to exercise tight control on the angle and origin of the light emitted, for example, using a microlens array or other light field shaping layer such as a parallax barrier, or combination thereof.

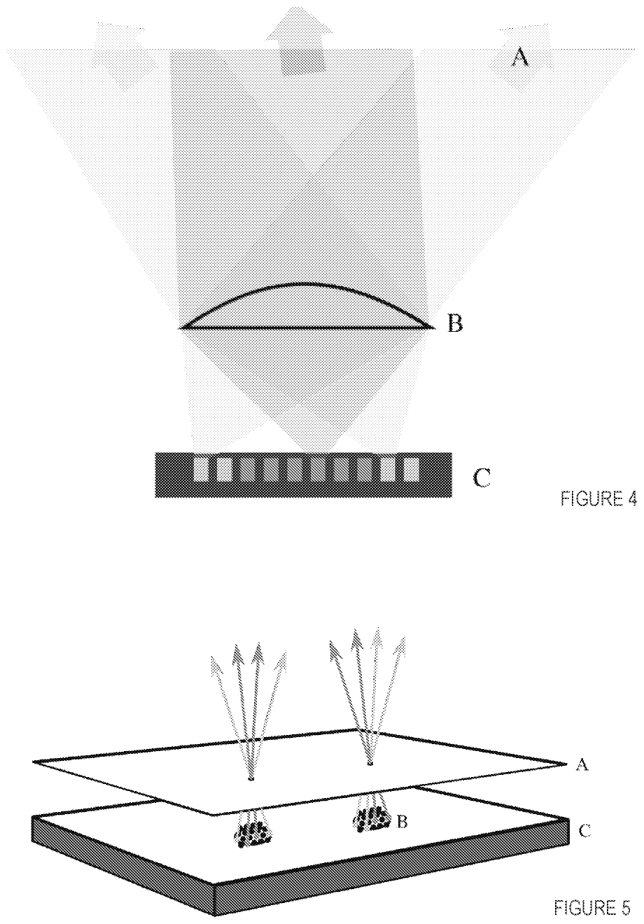

Following with the example of a microlens array, FIG. 4 schematically illustrates a single light field pixel defined by a convex microlens (B) disposed at its focus from a corresponding subset of pixels in an LCD display (C) to produce a substantially collimated beam of light emitted by these pixels, whereby the direction of the beam is controlled by the location of the pixel(s) relative to the microlens. The single light field pixel produces a beam similar to that shown in FIG. 3C where the outside rays are lighter and the majority inside rays are darker. The LCD display (C) emits light which hits the microlens (B) and it results in a beam of substantially collimated light (A).

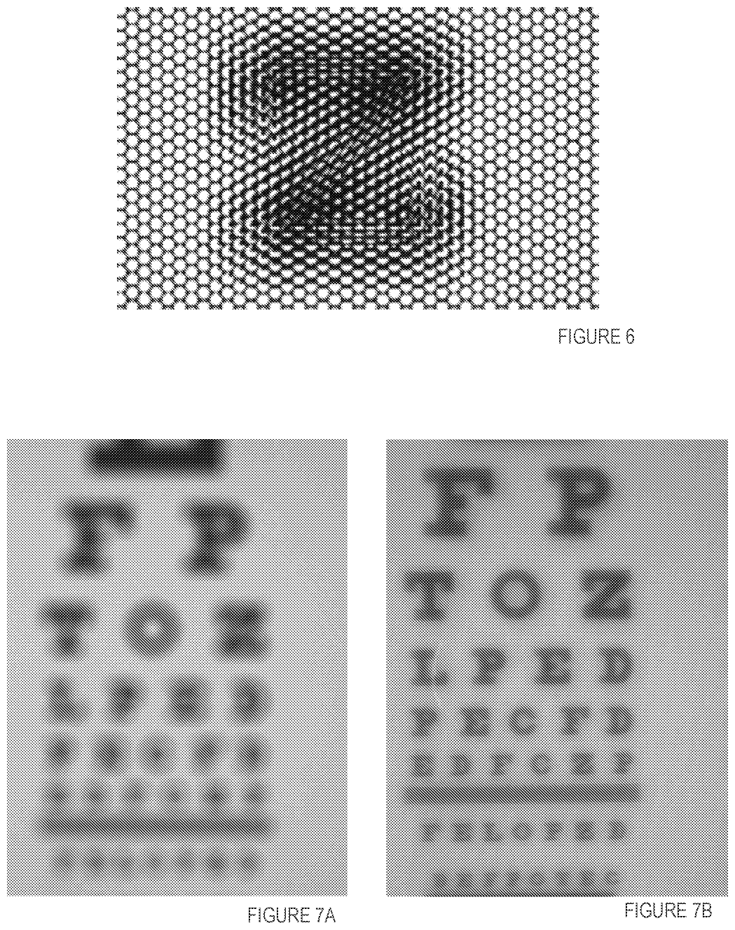

Accordingly, upon predictably aligning a particular microlens array with a pixel array, a designated “circle” of pixels will correspond with each microlens and be responsible for delivering light to the pupil through that lens. FIG. 5 schematically illustrates an example of a light field display assembly in which a microlens array (A) sits above an LCD display on a cellphone (C) to have pixels (B) emit light through the microlens array. A ray-tracing algorithm can thus be used to produce a pattern to be displayed on the pixel array below the microlens in order to create the desired virtual image that will effectively correct for the viewer's reduced visual acuity. FIG. 6 provides an example of such a pattern for the letter “Z”. Examples of such ray-tracing algorithms are discussed below.

As will be detailed further below, the separation between the microlens array and the pixel array as well as the pitch of the lenses can be selected as a function of various operating characteristics, such as the normal or average operating distance of the display, and/or normal or average operating ambient light levels.

Further, as producing a light field with angular resolution sufficient for accommodation correction over the full viewing ‘zone’ of a display would generally require an astronomically high pixel density, instead, a correct light field can be produced, in some embodiments, only at or around the location of the user's pupils. To do so, the light field display can be paired with pupil tracking technology to track a location of the user's eyes/pupils relative to the display. The display can then compensate for the user's eye location and produce the correct virtual image, for example, in real time.

In some embodiments, the light field display can render dynamic images at over 30 frames per second on the hardware in a smartphone.

In some embodiments, the light field display can display a virtual image at optical infinity, meaning that any level of accommodation-based presbyopia (e.g. first order) can be corrected for.

In some further embodiments, the light field display can both push the image back or forward, thus allowing for selective image corrections for both hyperopia (far-sightedness) and myopia (nearsightedness). This will be further discussed below in the context of a light field vision testing (e.g. refractor/phoropter) device using the light field display.

In order to demonstrate a working light field solution, and in accordance with one embodiment, the following test was set up. A camera was equipped with a simple lens, to simulate the lens in a human eye and the aperture was set to simulate a normal pupil diameter. The lens was focused to 50 cm away and a phone was mounted 25 cm away. This would approximate a user whose minimal seeing distance is 50 cm and is attempting to use a phone at 25 cm.

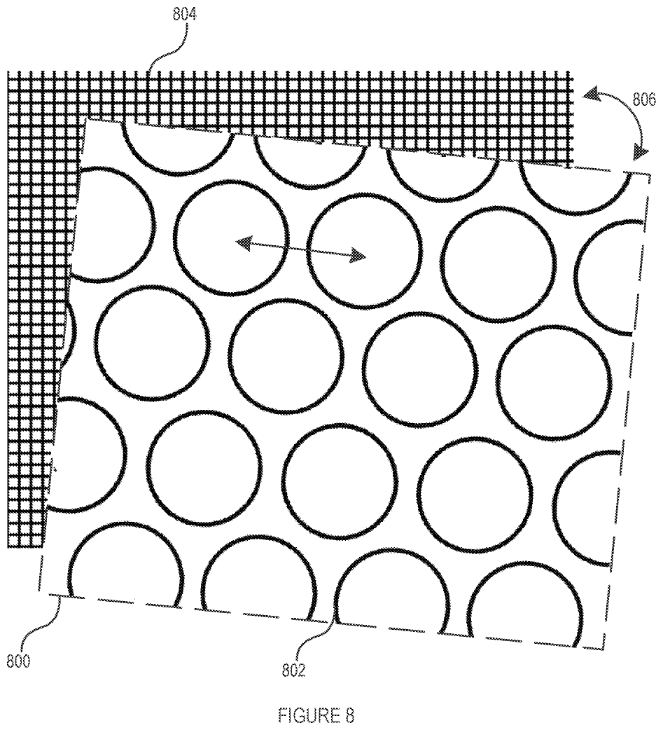

With reading glasses, +2.0 diopters would be necessary for the vision correction. A scaled Snellen chart was displayed on the cellphone and a picture was taken, as shown in FIG. 7A. Using the same cellphone, but with a light field assembly in front that uses that cellphone's pixel array, a virtual image compensating for the lens focus is displayed. A picture was again taken, as shown in FIG. 7B, showing a clear improvement.

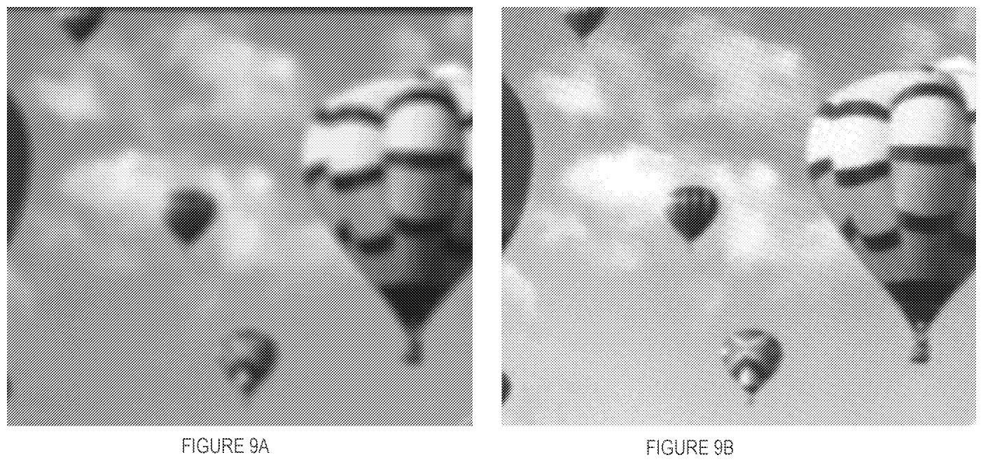

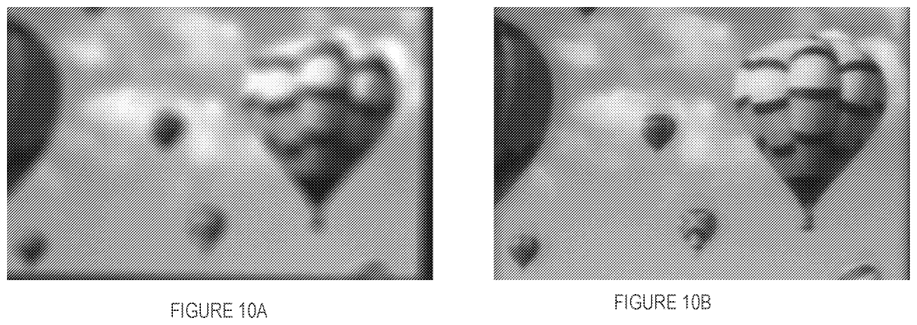

FIGS. 9A and 9B provide another example of results achieved using an exemplary embodiment, in which a colour image was displayed on the LCD display of a Sony™ Xperia™ XZ Premium phone (reported screen resolution of 3840×2160 pixels with 16:9 ratio and approximately 807 pixel-per-inch (ppi) density) without image correction (FIG. 9A) and with image correction through a square fused silica microlens array set at a 2 degree angle relative to the screen's square pixel array and defined by microlenses having a 7.0 mm focus and 200 μm pitch. In this example, the camera lens was again focused at 50 cm with the phone positioned 30 cm away. Another microlens array was used to produce similar results, and consisted of microlenses having a 10.0 mm focus and 150 μm pitch.

FIGS. 10A and 10B provide yet another example or results achieved using an exemplary embodiment, in which a colour image was displayed on the LCD display of a the Sony™ Xperia™ XZ Premium phone without image correction (FIG. 10A) and with image correction through a square fused silica microlens array set at a 2 degree angle relative to the screen's square pixel array and defined by microlenses having a 10.0 mm focus and 150 μm pitch. In this example, the camera lens was focused at 66 cm with the phone positioned 40 cm away.

Accordingly, a display device as described above and further exemplified below, can be configured to render a corrected image via the light field shaping layer that accommodates for the user's visual acuity. By adjusting the image correction in accordance with the user's actual predefined, set or selected visual acuity level, different users and visual acuity may be accommodated using a same device configuration. That is, in one example, by adjusting corrective image pixel data to dynamically adjust a virtual image distance below/above the display as rendered via the light field shaping layer, different visual acuity levels may be accommodated.

As will be appreciated by the skilled artisan, different image processing techniques may be considered, such as those introduced above and taught by Pamplona and/or Huang, for example, which may also influence other light field parameters to achieve appropriate image correction, virtual image resolution, brightness and the like.

With reference to FIG. 8, and in accordance with one embodiment, a microlens array configuration will now be described, in accordance with another embodiment, to provide light field shaping elements in a corrective light field implementation. In this embodiment, the microlens array 800 is defined by a hexagonal array of microlenses 802 disposed so to overlay a corresponding square pixel array 804. In doing so, while each microlens 802 can be aligned with a designated subset of pixels to produce light field pixels as described above, the hexagonal-to-square array mismatch can alleviate certain periodic optical artifacts that may otherwise be manifested given the periodic nature of the optical elements and principles being relied upon to produce the desired optical image corrections. Conversely, a square microlens array may be favoured when operating a digital display comprising a hexagonal pixel array.

In some embodiments, as illustrated in FIG. 8, the microlens array 800 may further or alternatively overlaid at an angle 806 relative to the underlying pixel array, which can further or alternatively alleviate period optical artifacts.

In yet some further or alternative embodiments, a pitch ratio between the microlens array and pixel array may be deliberately selected to further or alternatively alleviate periodic optical artifacts. For example, a perfectly matched pitch ratio (i.e. an exact integer number of display pixels per microlens) is most likely to induce periodic optical artifacts, whereas a pitch ratio mismatch can help reduce such occurrences. Accordingly, in some embodiments, the pitch ratio will be selected to define an irrational number, or at least, an irregular ratio, so to minimize periodic optical artifacts. For instance, a structural periodicity can be defined so to reduce the number of periodic occurrences within the dimensions of the display screen at hand, e.g. ideally selected so to define a structural period that is greater than the size of the display screen being used.

While this example is provided within the context of a microlens array, similar structural design considerations may be applied within the context of a parallax barrier, diffractive barrier or combination thereof.

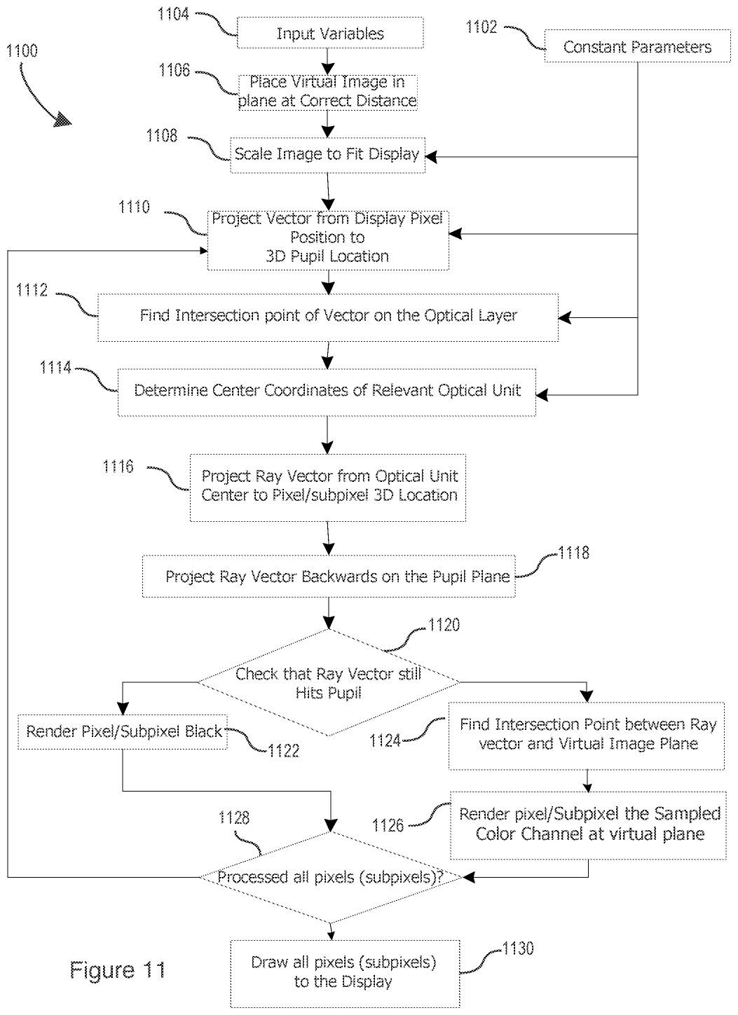

With reference to FIGS. 11 to 13, and in accordance with one embodiment, an exemplary computationally implemented ray-tracing method for rendering an adjusted image via an array of light field shaping elements, in this example provided by a light field shaping layer (LFSL) disposed relative to a set of underlying display pixels, that accommodates for the user's reduced visual acuity will now be described. In this example, for illustrative purposes, adjustment of a single image (i.e. the image as whole) is being implemented without consideration for distinct image portions. Further examples below will specifically address modification of the following example for adaptively adjusting distinct image portions.

In this exemplary embodiment, a set of constant parameters 1102 may be pre-determined. These may include, for example, any data that are not expected to significantly change during a user's viewing session, for instance, which are generally based on the physical and functional characteristics of the display for which the method is to be implemented, as will be explained below. Similarly, every iteration of the rendering algorithm may use a set of input variables 1104 which are expected to change either at each rendering iteration or at least between each user's viewing session.

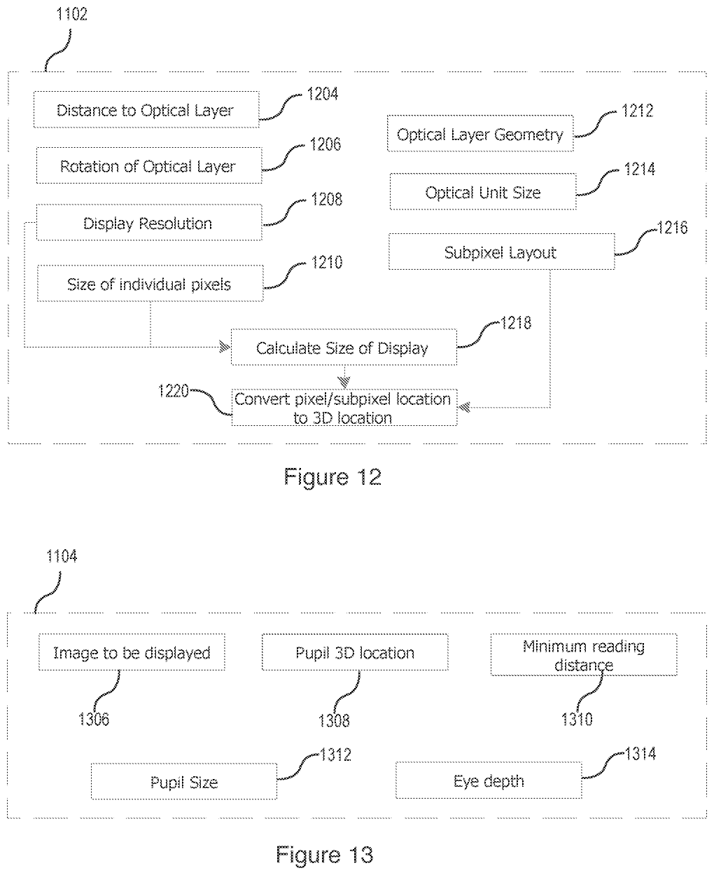

As illustrated in FIG. 12, the list of constant parameters 1102 may include, without limitations, the distance 1204 between the display and the LFSL, the in-plane rotation angle 1206 between the display and LFSL frames of reference, the display resolution 1208, the size of each individual pixel 1210, the optical LFSL geometry 1212, the size of each optical element 1214 within the LFSL and optionally the subpixel layout 1216 of the display. Moreover, both the display resolution 1208 and the size of each individual pixel 1210 may be used to pre-determine both the absolute size of the display in real units (i.e. in mm) and the three-dimensional position of each pixel within the display. In some embodiments where the subpixel layout 1216 is available, the position within the display of each subpixel may also be pre-determined. These three-dimensional location/positions are usually calculated using a given frame of reference located somewhere within the plane of the display, for example a corner or the middle of the display, although other reference points may be chosen. Concerning the optical layer geometry 1212, different geometries may be considered, for example a hexagonal geometry such as the one shown in FIG. 8. Finally, by combining the distance 1204, the rotation angle 1206, and the geometry 1212 with the optical element size 1214, it is possible to similarly pre-determine the three-dimensional location/position of each optical element center with respect to the display's same frame of reference.

FIG. 13 meanwhile illustratively lists an exemplary set of input variables 1104 for method 1100, which may include any input data fed into method 1100 that may reasonably change during a user's single viewing session, and may thus include without limitation: the image(s) to be displayed 1306 (e.g. pixel data such as on/off, colour, brightness, etc.), the three-dimensional pupil location 1308 (e.g. in embodiments implementing active eye/pupil tracking methods) and/or pupil size 1312 and the minimum reading distance 1310 (e.g. one or more parameters representative of the user's reduced visual acuity or condition). In some embodiments, the eye depth 1314 may also be used. The image data 1306, for example, may be representative of one or more digital images to be displayed with the digital pixel display. This image may generally be encoded in any data format used to store digital images known in the art. In some embodiments, images 1306 to be displayed may change at a given framerate.

The pupil location 1308, in one embodiment, is the three-dimensional coordinates of at least one the user's pupils' center with respect to a given reference frame, for example a point on the device or display. This pupil location 1308 may be derived from any eye/pupil tracking method known in the art. In some embodiments, the pupil location 1308 may be determined prior to any new iteration of the rendering algorithm, or in other cases, at a lower framerate. In some embodiments, only the pupil location of a single user's eye may be determined, for example the user's dominant eye (i.e. the one that is primarily relied upon by the user). In some embodiments, this position, and particularly the pupil distance to the screen may otherwise or additionally be rather approximated or adjusted based on other contextual or environmental parameters, such as an average or preset user distance to the screen (e.g. typical reading distance for a given user or group of users; stored, set or adjustable driver distance in a vehicular environment; etc.).

In the illustrated embodiment, the minimum reading distance 1310 is defined as the minimal focus distance for reading that the user's eye(s) may be able to accommodate (i.e. able to view without discomfort). In some embodiments, different values of the minimum reading distance 1310 associated with different users may be entered, for example, as can other adaptive vision correction parameters be considered depending on the application at hand and vision correction being addressed. In some embodiments, minimum reading distance 1310 may be derived from an eye prescription (e.g. glasses prescription or contact prescription) or similar. It may, for example, correspond to the near point distance corresponding to the uncorrected user's eye, which can be calculated from the prescribed corrective lens power assuming that the targeted near point was at 25 cm.

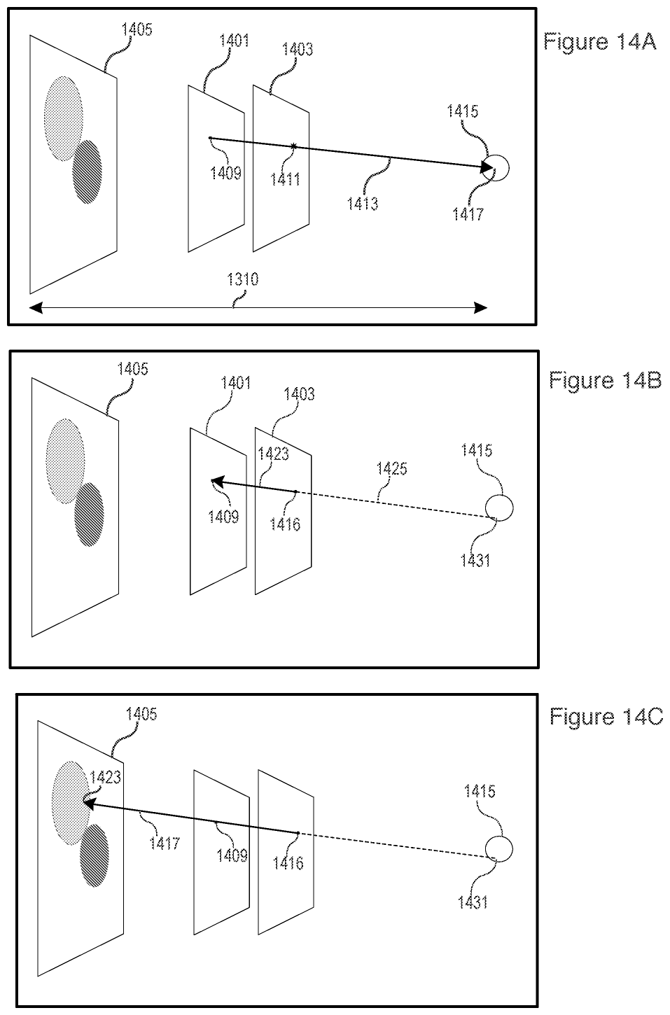

With added reference to FIGS. 14A to 14C, once parameters 1102 and variables 1104 have been set, the method of FIG. 11 then proceeds with step 1106, in which the minimum reading distance 1310 (and/or related parameters) is used to compute the position of a virtual (adjusted) image plane 1405 with respect to the device's display, followed by step 1108 wherein the size of image 1306 is scaled within the image plane 1405 to ensure that it correctly fills the pixel display 1401 when viewed by the distant user. This is illustrated in FIG. 14A, which shows a diagram of the relative positioning of the user's pupil 1415, the light field shaping layer 1403, the pixel display 1401 and the virtual image plane 1405. In this example, the size of image 1306 in image plane 1405 is increased to avoid having the image as perceived by the user appear smaller than the display's size.

An exemplary ray-tracing methodology is described in steps 1110 to 1128 of FIG. 11, at the end of which the output color of each pixel of pixel display 1401 is known so as to virtually reproduce the light field emanating from an image 1306 positioned at the virtual image plane 1405. In FIG. 11, these steps are illustrated in a loop over each pixel in pixel display 1401, so that each of steps 1110 to 1126 describes the computations done for each individual pixel. However, in some embodiments, these computations need not be executed sequentially, but rather, steps 1110 to 1128 may be executed in parallel for each pixel or a subset of pixels at the same time. Indeed, as will be discussed below, this exemplary method is well suited to vectorization and implementation on highly parallel processing architectures such as GPUs.

As illustrated in FIG. 14A, in step 1110, for a given pixel 1409 in pixel display 1401, a trial vector 1413 is first generated from the pixel's position to the center position 1417 of pupil 1415. This is followed in step 1112 by calculating the intersection point 1411 of vector 1413 with the LFSL 1403.

The method then finds, in step 1114, the coordinates of the center 1416 of the LFSL optical element closest to intersection point 1411. This step may be computationally intensive and will be discussed in more depth below. Once the position of the center 1416 of the optical element is known, in step 1116, a normalized unit ray vector is generated from drawing and normalizing a vector 1423 drawn from center position 1416 to pixel 1409. This unit ray vector generally approximates the direction of the light field emanating from pixel 1409 through this particular light field element, for instance, when considering a parallax barrier aperture or lenslet array (i.e. where the path of light travelling through the center of a given lenslet is not deviated by this lenslet). Further computation may be required when addressing more complex light shaping elements, as will be appreciated by the skilled artisan. The direction of this ray vector will be used to find the portion of image 1306, and thus the associated color, represented by pixel 1409. But first, in step 1118, this ray vector is projected backwards to the plane of pupil 1415, and then in step 1120, the method verifies that the projected ray vector 1425 is still within pupil 1415 (i.e. that the user can still “see” it). Once the intersection position, for example location 1431 in FIG. 14B, of projected ray vector 1425 with the pupil plane is known, the distance between the pupil center 1417 and the intersection point 1431 may be calculated to determine if the deviation is acceptable, for example by using a pre-determined pupil size and verifying how far the projected ray vector is from the pupil center.

If this deviation is deemed to be too large (i.e. light emanating from pixel 1409 channeled through optical element 1416 is not perceived by pupil 1415), then in step 1122, the method flags pixel 1409 as unnecessary and to simply be turned off or render a black color. Otherwise, as shown in FIG. 14C, in step 1124, the ray vector is projected once more towards virtual image plane 1405 to find the position of the intersection point 1423 on image 1306. Then in step 1126, pixel 1409 is flagged as having the color value associated with the portion of image 1306 at intersection point 1423.

In some embodiments, method 1100 is modified so that at step 1120, instead of having a binary choice between the ray vector hitting the pupil or not, one or more smooth interpolation function (i.e. linear interpolation, Hermite interpolation or similar) are used to quantify how far or how close the intersection point 1431 is to the pupil center 1417 by outputting a corresponding continuous value between 1 or 0. For example, the assigned value is equal to 1 substantially close to pupil center 1417 and gradually change to 0 as the intersection point 1431 substantially approaches the pupil edges or beyond. In this case, the branch containing step 1122 is ignored and step 1220 continues to step 1124. At step 1126, the pixel color value assigned to pixel 1409 is chosen to be somewhere between the full color value of the portion of image 1306 at intersection point 1423 or black, depending on the value of the interpolation function used at step 1120 (1 or 0).

In yet other embodiments, pixels found to illuminate a designated area around the pupil may still be rendered, for example, to produce a buffer zone to accommodate small movements in pupil location, for example, or again, to address potential inaccuracies, misalignments or to create a better user experience.

In some embodiments, steps 1118, 1120 and 1122 may be avoided completely, the method instead going directly from step 1116 to step 1124. In such an exemplary embodiment, no check is made that the ray vector hits the pupil or not, but instead the method assumes that it always does.

Once the output colors of all pixels have been determined, these are finally rendered in step 1130 by pixel display 1401 to be viewed by the user, therefore presenting a light field corrected image. In the case of a single static image, the method may stop here. However, new input variables may be entered and the image may be refreshed at any desired frequency, for example because the user's pupil moves as a function of time and/or because instead of a single image a series of images are displayed at a given framerate.

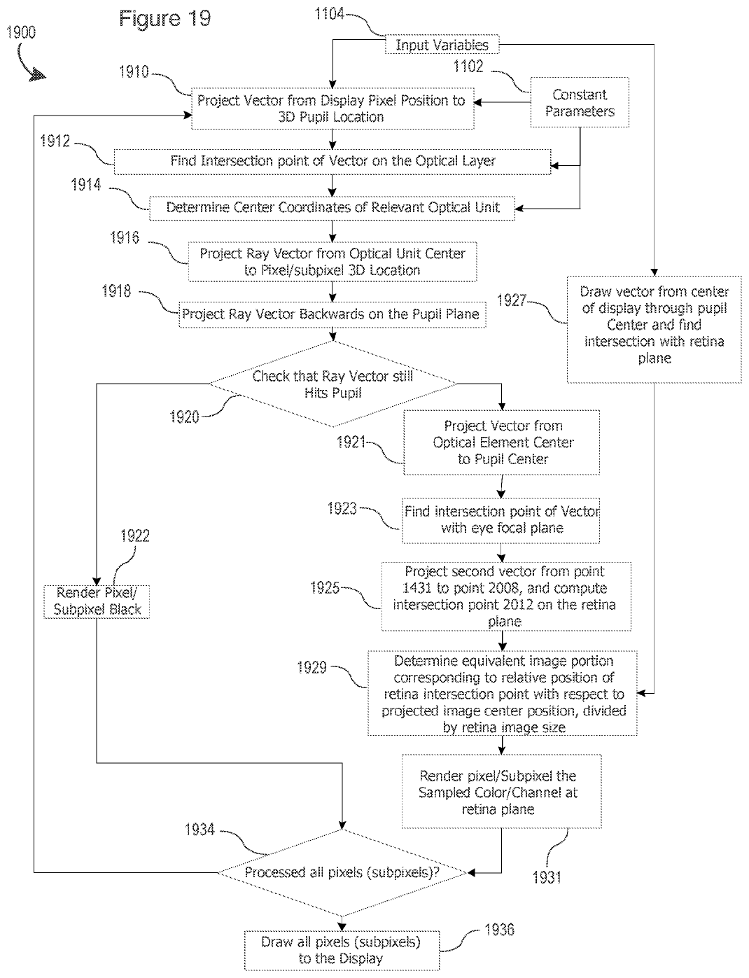

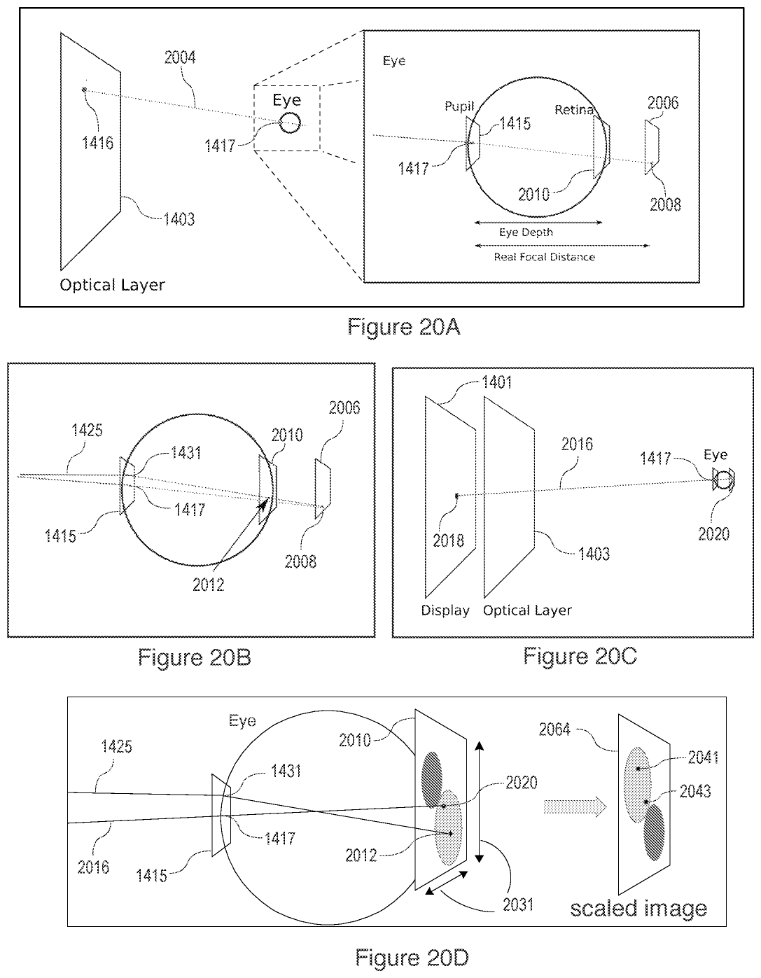

With reference to FIGS. 19 and 20A to 20D, and in accordance with one embodiment, another exemplary computationally implemented ray-tracing method for rendering an adjusted image via the light field shaping layer (LFSL) that accommodates for the user's reduced visual acuity, for example, will now be described. Again, for illustrative purposes, in this example, adjustment of a single image (i.e. the image as whole) is being implemented without consideration for distinct image portions. Further examples below will specifically address modification of the following example for adaptively adjusting distinct image portions.

In this embodiment, the adjusted image portion associated with a given pixel/subpixel is computed (mapped) on the retina plane instead of the virtual image plane considered in the above example, again in order to provide the user with a designated image perception adjustment. Therefore, the currently discussed exemplary embodiment shares some steps with the method of FIG. 11. Indeed, a set of constant parameters 1102 may also be pre-determined. These may include, for example, any data that are not expected to significantly change during a user's viewing session, for instance, which are generally based on the physical and functional characteristics of the display for which the method is to be implemented, as will be explained below. Similarly, every iteration of the rendering algorithm may use a set of input variables 1104 which are expected to change either at each rendering iteration or at least between each user viewing session. The list of possible variables and constants is substantially the same as the one disclosed in FIGS. 12 and 13 and will thus not be replicated here.

Once parameters 1102 and variables 1104 have been set, this second exemplary ray-tracing methodology proceeds from steps 1910 to 1936, at the end of which the output color of each pixel of the pixel display is known so as to virtually reproduce the light field emanating from an image perceived to be positioned at the correct or adjusted image distance, in one example, so to allow the user to properly focus on this adjusted image (i.e. having a focused image projected on the user's retina) despite a quantified visual aberration. In FIG. 19, these steps are illustrated in a loop over each pixel in pixel display 1401, so that each of steps 1910 to 1934 describes the computations done for each individual pixel. However, in some embodiments, these computations need not be executed sequentially, but rather, steps 1910 to 1934 may be executed in parallel for each pixel or a subset of pixels at the same time. Indeed, as will be discussed below, this second exemplary method is also well suited to vectorization and implementation on highly parallel processing architectures such as GPUs.

Referencing once more FIG. 14A, in step 1910 (as in step 1110), for a given pixel in pixel display 1401, a trial vector 1413 is first generated from the pixel's position to pupil center 1417 of the user's pupil 1415. This is followed in step 1912 by calculating the intersection point of vector 1413 with optical layer 1403.

From there, in step 1914, the coordinates of the optical element center 1416 closest to intersection point 1411 are determined. This step may be computationally intensive and will be discussed in more depth below. As shown in FIG. 14B, once the position of the optical element center 1416 is known, in step 1916, a normalized unit ray vector is generated from drawing and normalizing a vector 1423 drawn from optical element center 1416 to pixel 1409. This unit ray vector generally approximates the direction of the light field emanating from pixel 1409 through this particular light field element, for instance, when considering a parallax barrier aperture or lenslet array (i.e. where the path of light travelling through the center of a given lenslet is not deviated by this lenslet). Further computation may be required when addressing more complex light shaping elements, as will be appreciated by the skilled artisan. In step 1918, this ray vector is projected backwards to pupil 1415, and then in step 1920, the method ensures that the projected ray vector 1425 is still within pupil 1415 (i.e. that the user can still “see” it). Once the intersection position, for example location 1431 in FIG. 14B, of projected ray vector 1425 with the pupil plane is known, the distance between the pupil center 1417 and the intersection point 1431 may be calculated to determine if the deviation is acceptable, for example by using a pre-determined pupil size and verifying how far the projected ray vector is from the pupil center.

Now referring to FIGS. 20A to 20D, steps 1921 to 1929 of method 1900 will be described. Once optical element center 1416 of the relevant optical unit has been determined, at step 1921, a vector 2004 is drawn from optical element center 1416 to pupil center 1417. Then, in step 1923, vector 2004 is projected further behind the pupil plane onto eye focal plane 2006 (location where any light rays originating from optical layer 1403 would be focused by the eye) to locate focal point 2008. For a user with perfect vision, focal plane 2006 would be located at the same location as retina plane 2010, but in this example, focal plane 2006 is located behind retina plane 2010, which would be expected for a user with some form of farsightedness. The position of focal plane 2006 may be derived from the user's minimum reading distance 1310, for example, by deriving therefrom the focal length of the user's eye. Other manually input or computationally or dynamically adjustable means may also or alternatively be considered to quantify this parameter.

The skilled artisan will note that any light ray originating from optical element center 1416, no matter its orientation, will also be focused onto focal point 2008, to a first approximation. Therefore, the location 2012 on retina plane 2010 onto which light entering the pupil at intersection point 1431 will converge may be approximated by drawing a straight line between intersection point 1431 where ray vector 1425 hits the pupil 1415 and focal point 2008 on focal plane 2006. The intersection of this line with retina plane 2010 (retina image point 2012) is thus the location on the user's retina corresponding to the image portion that will be reproduced by corresponding pixel 1409 as perceived by the user. Therefore, by comparing the relative position of retina point 2012 with the overall position of the projected image on the retina plane 2010, the relevant adjusted image portion associated with pixel 1409 may be computed.

To do so, at step 1927, the corresponding projected image center position on retina plane 2010 is calculated. Vector 2016 is generated originating from the center position of display 1401 (display center position 2018) and passing through pupil center 1417. Vector 2016 is projected beyond the pupil plane onto retina plane 2010, wherein the associated intersection point gives the location of the corresponding retina image center 2020 on retina plane 2010. The skilled technician will understand that step 1927 could be performed at any moment prior to step 1929, once the relative pupil center location 1417 is known in input variables step 1904. Once image center 2020 is known, one can then find the corresponding image portion of the selected pixel/subpixel at step 1929 by calculating the x/y coordinates of retina image point 2012 relative to retina image center 2020 on the retina, scaled to the x/y retina image size 2031.

This retina image size 2031 may be computed by calculating the magnification of an individual pixel on retina plane 2010, for example, which may be approximately equal to the x or y dimension of an individual pixel multiplied by the eye depth 1314 and divided by the absolute value of the distance to the eye (i.e. the magnification of pixel image size from the eye lens). Similarly, for comparison purposes, the input image is also scaled by the image x/y dimensions to produce a corresponding scaled input image 2064. Both the scaled input image and scaled retina image should have a width and height between −0.5 to 0.5 units, enabling a direct comparison between a point on the scaled retina image 2010 and the corresponding scaled input image 2064, as shown in FIG. 20D.

From there, the image portion position 2041 relative to retina image center position 2043 in the scaled coordinates (scaled input image 2064) corresponds to the inverse (because the image on the retina is inverted) scaled coordinates of retina image point 2012 with respect to retina image center 2020. The associated color with image portion position 2041 is therefrom extracted and associated with pixel 1409.

In some embodiments, method 1900 may be modified so that at step 1920, instead of having a binary choice between the ray vector hitting the pupil or not, one or more smooth interpolation function (i.e. linear interpolation, Hermite interpolation or similar) are used to quantify how far or how close the intersection point 1431 is to the pupil center 1417 by outputting a corresponding continuous value between 1 or 0. For example, the assigned value is equal to 1 substantially close to pupil center 1417 and gradually change to 0 as the intersection point 1431 substantially approaches the pupil edges or beyond. In this case, the branch containing step 1122 is ignored and step 1920 continues to step 1124. At step 1931, the pixel color value assigned to pixel 1409 is chosen to be somewhere between the full color value of the portion of image 1306 at intersection point 1423 or black, depending on the value of the interpolation function used at step 1920 (1 or 0).

In yet other embodiments, pixels found to illuminate a designated area around the pupil may still be rendered, for example, to produce a buffer zone to accommodate small movements in pupil location, for example, or again, to address potential inaccuracies or misalignments.

Once the output colors of all pixels in the display have been determined (check at step 1934 is true), these are finally rendered in step 1936 by pixel display 1401 to be viewed by the user, therefore presenting a light field corrected image. In the case of a single static image, the method may stop here. However, new input variables may be entered and the image may be refreshed at any desired frequency, for example because the user's pupil moves as a function of time and/or because instead of a single image a series of images are displayed at a given framerate.

As will be appreciated by the skilled artisan, selection of the adjusted image plane onto which to map the input image in order to adjust a user perception of this input image allows for different ray tracing approaches to solving a similar challenge, that is of creating an adjusted image using the light field display that can provide an adjusted user perception, such as addressing a user's reduce visual acuity. While mapping the input image to a virtual image plane set at a designated minimum (or maximum) comfortable viewing distance can provide one solution, the alternate solution may allow accommodation of different or possibly more extreme visual aberrations. For example, where a virtual image is ideally pushed to infinity (or effectively so), computation of an infinite distance becomes problematic. However, by designating the adjusted image plane as the retinal plane, the illustrative process of FIG. 19 can accommodate the formation of a virtual image effectively set at infinity without invoking such computational challenges. Likewise, while first order aberrations are illustratively described with reference to FIG. 19, higher order or other optical anomalies may be considered within the present context, whereby a desired retinal image is mapped out and traced while accounting for the user's optical aberration(s) so to compute adjusted pixel data to be rendered in producing that image. These and other such considerations should be readily apparent to the skilled artisan.

While the computations involved in the above described ray-tracing algorithms (steps 1110 to 1128 of FIG. 11 or steps 1920 to 1934 of FIG. 19) may be done on general CPUs, it may be advantageous to use highly parallel programming schemes to speed up such computations. While in some embodiments, standard parallel programming libraries such as Message Passing Interface (MPI) or OPENMP may be used to accelerate the light field rendering via a general-purpose CPU, the light field computations described above are especially tailored to take advantage of graphical processing units (GPU), which are specifically tailored for massively parallel computations. Indeed, modern GPU chips are characterized by the very large number of processing cores, and an instruction set that is commonly optimized for graphics. In typical use, each core is dedicated to a small neighborhood of pixel values within an image, e.g., to perform processing that applies a visual effect, such as shading, fog, affine transformation, etc. GPUs are usually also optimized to accelerate exchange of image data between such processing cores and associated memory, such as RGB frame buffers. Furthermore, smartphones are increasingly being equipped with powerful GPUs to speed the rendering of complex screen displays, e.g., for gaming, video, and other image-intensive applications. Several programming frameworks and languages tailored for programming on GPUs include, but are not limited to, CUDA, OpenCL, OpenGL Shader Language (GLSL), High-Level Shader Language (HLSL) or similar. However, using GPUs efficiently may be challenging and thus require creative steps to leverage their capabilities, as will be discussed below.

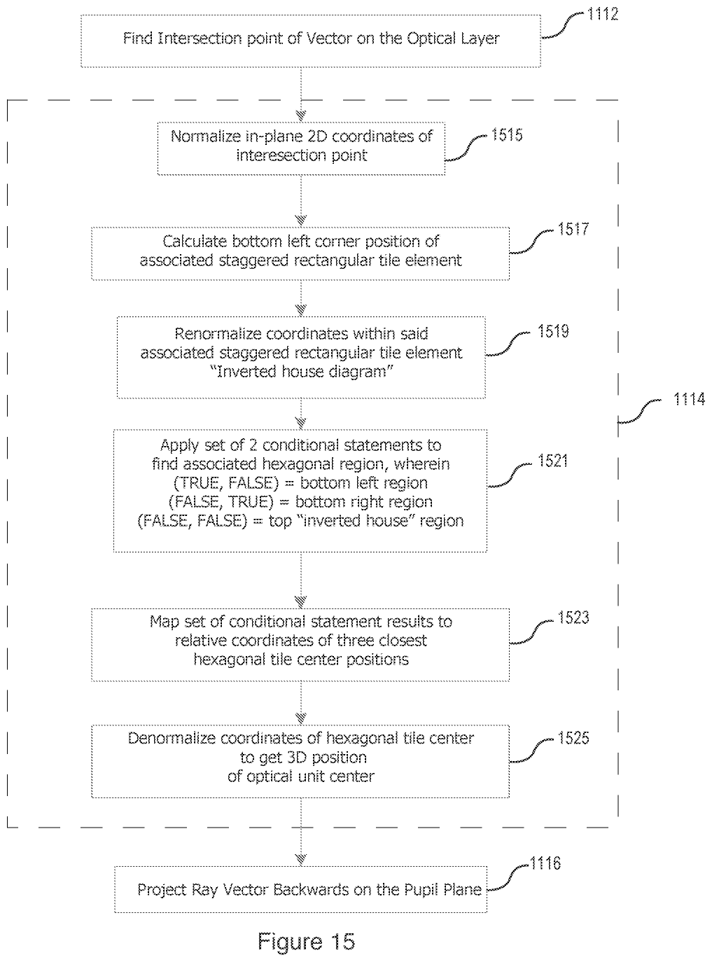

With reference to FIGS. 15 to 18C and in accordance with one exemplary embodiment, an exemplary process for computing the center position of an associated light field shaping element in the ray-tracing process of FIG. 11 (or FIG. 19) will now be described. The series of steps are specifically tailored to avoid code branching, so as to be increasingly efficient when run on GPUs (i.e. to avoid so called “warp divergence”). Indeed, with GPUs, because all the processors must execute identical instructions, divergent branching can result in reduced performance.

With reference to FIG. 15, and in accordance with one embodiment, step 1114 of FIG. 11 is expanded to include steps 1515 to 1525. A similar discussion can readily be made in respect of step 1914 of FIG. 19, and thus need not be explicitly detailed herein. The method receives from step 1112 the 2D coordinates of the intersection point 1411 (illustrated in FIG. 14A) of the trial vector 1413 with optical layer 1403. As discussed with respect to the exemplary embodiment of FIG. 8, there may be a difference in orientation between the frames of reference of the optical layer (hexagonal array of microlenses 802 in FIG. 8, for example) and of the corresponding pixel display (square pixel array 804 in FIG. 8, for example). This is why, in step 1515, these input intersection coordinates, which are initially calculated from the display's frame of reference, may first be rotated to be expressed from the light field shaping layer's frame of reference and optionally normalized so that each individual light shaping element has a width and height of 1 unit. The following description will be equally applicable to any light field shaping layer having a hexagonal geometry like the exemplary embodiment of FIG. 8. Note however that the method steps 1515 to 1525 described herein may be equally applied to any kind of light field shaping layer sharing the same geometry (i.e. not only a microlens array, but pinhole arrays as well, etc.). Likewise, while the following example is specific to an exemplary hexagonal array of LFSL elements definable by a hexagonal tile array of regular hexagonal tiles, other geometries may also benefit from some or all of the features and/or advantages of the herein-described and illustrated embodiments. For example, different hexagonal LFSL element arrays, such as stretched/elongated, skewed and/or rotated arrays may be considered, as can other nestled array geometries in which adjacent rows and/or columns of the LFSL array at least partially “overlap” or inter-nest. For instance, as will be described further below, hexagonal arrays and like nestled array geometries will generally provide for a commensurately sized rectangular/square tile of an overlaid rectangular/square array or grid to naturally encompass distinct regions as defined by two or more adjacent underlying nestled array tiles, which can be used to advantage in the examples provided below. In yet other embodiments, the processes discussed herein may be applied to rectangular and/or square LFSL element arrays. Other LFSL element array geometries may also be considered, as will be appreciated by the skilled artisan upon reading of the following example, without departing from the general scope and nature of the present disclosure.

For hexagonal geometries, as illustrated in FIGS. 16A and 16B, the hexagonal symmetry of the light field shaping layer 1403 may be represented by drawing an array of hexagonal tiles 1601, each centered on their respective light field shaping element, so that the center of a hexagonal tile element is more or less exactly the same as the center position of its associated light field shaping element. Thus, the original problem is translated to a slightly similar one whereby one now needs to find the center position 1615 of the associated hexagonal tile 1609 closest to the intersection point 1411, as shown in FIG. 16B.

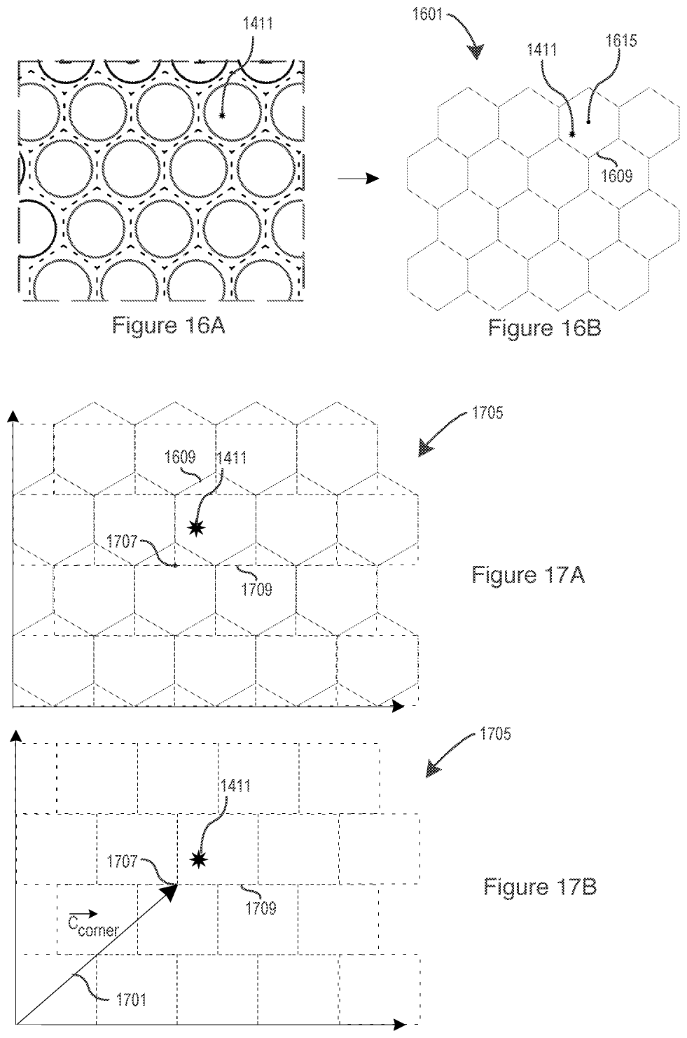

To solve this problem, the array of hexagonal tiles 1601 may be superimposed on or by a second array of staggered rectangular tiles 1705, in such a way as to make an “inverted house” diagram within each rectangle, as clearly illustrated in FIG. 17A, namely defining three linearly segregated tile regions for each rectangular tile, one region predominantly associated with a main underlying hexagonal tile, and two other opposed triangular regions associated with adjacent underlying hexagonal tiles. In doing so, the nestled hexagonal tile geometry is translated to a rectangular tile geometry having distinct linearly segregated tile regions defined therein by the edges of underlying adjacently disposed hexagonal tiles. Again, while regular hexagons are used to represent the generally nestled hexagonal LFSL element array geometry, other nestled tile geometries may be used to represent different nestled element geometries. Likewise, while a nestled array is shown in this example, different staggered or aligned geometries may also be used, in some examples, in some respects, with reduced complexity, as further described below.

Furthermore, while this particular example encompasses the definition of linearly defined tile region boundaries, other boundary types may also be considered provided they are amenable to the definition of one or more conditional statements, as illustrated below, that can be used to output a corresponding set of binary or Boolean values that distinctly identify a location of a given point within one or another of these regions, for instance, without invoking, or by limiting, processing demands common to branching or looping decision logics/trees/statements/etc.

Following with hexagonal example, to locate the associated hexagon tile center 1615 closest to the intersection point 1411, in step 1517, the method first computes the 2D position of the bottom left corner 1707 of the associated (normalized) rectangular tile element 1709 containing intersection point 1411, as shown in FIG. 17B, which can be calculated without using any branching statements by the following two equations (here in normalized coordinates wherein each rectangle has a height and width of one unit):

$$\vec{t} = ( floor ( {uv}_{y} ) , 0 )$$

$${\vec{C}}_{corner} = ( \vec{u v} + \vec{t} ) - \vec{t}$$

where \vec{uv} is the position vector of intersection point 1411 in the common frame of reference of the hexagonal and staggered rectangular tile arrays, and the floor( ) function returns the greatest integer less than or equal to each of the xy coordinates of \vec{uv}.

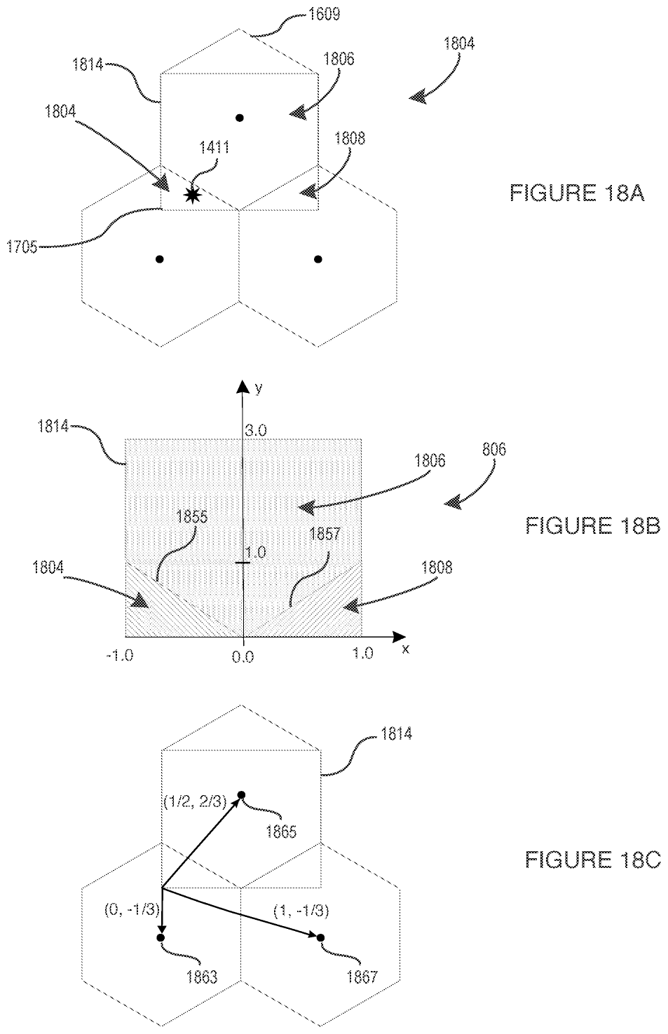

Once the position of lower left corner [symbol] 1707, indicated by vector \vec{C}_{corner} 1701, of the associated rectangular element 1814 containing the intersection point 1411 is known, three regions 1804, 1806 and 1807 within this rectangular element 1814 may be distinguished, as shown in FIGS. 18A to 18C. Each region is associated with a different hexagonal tile, as shown in FIG. 18A, namely, each region is delineated by the linear boundaries of adjacent underlying hexagonal tiles to define one region predominantly associated with a main hexagonal tile, and two opposed triangular tiles defined by adjacent hexagonal tiles on either side of this main tile. As will be appreciated by the skilled artisan, different hexagonal or nestled tile geometries will result in the delineation of different rectangular tile region shapes, as will different boundary profiles (straight vs. curved) will result in the definition of different boundary value statements, defined further below.

Continuing with the illustrated example, in step 1519, the coordinates within associated rectangular tile 1814 are again rescaled, as shown on the axis of FIG. 18B, so that the intersection point's location, within the associated rectangular tile, is now represented in the rescaled coordinates by a vector d where each of its x and y coordinates are given by:

$${d}_{x} = 2 * ( u {v}_{x} - {C}_{{corner}_{x}} ) - 1$$

$${d}_{y} = 3 * ( u {v}_{y} - {C}_{{corner}_{y}} ) .$$

Thus, the possible x and y values of the position of intersection point 1411 within associated rectangular tile 1814 are now contained within −1<x<1 and 0<y<3. This will make the next step easier to compute.

To efficiently find the region encompassing a given intersection point in these rescaled coordinates, the fact that, within the rectangular element 1814, each region is separated by a diagonal line is used. For example, this is illustrated in FIG. 18B, wherein the lower left region 1804 is separated from the middle “inverted house” region 1806 and lower right region 1808 by a downward diagonal line 1855, which in the rescaled coordinates of FIG. 18B, follows the simple equation y=−x. Thus, all points where x<−y are located in the lower left region. Similarly, the lower right region 1808 is separated from the other two regions by a diagonal line 1857 described by the equation y<x. Therefore, in step 1521, the associated region containing the intersection point is evaluated by using these two simple conditional statements. The resulting set of two Boolean values will thus be specific to the region where the intersection point is located. For example, the checks (caseL=x<y, caseR=y<x) will result in the values (caseL=true, caseR=false), (caseL=false, caseR=true) and (caseL=false, caseR=false) for intersection points located in the lower left region 1804, lower right region 1808 and middle region 1806, respectively. One may then convert these Boolean values to floating points values, wherein usually in most programming languages true/false Boolean values are converted into 1.0/0.0 floating point values. Thus, one obtains the set (caseL, caseR) of values of (1.0, 0.0), (0.0, 1.0) or (0.0, 0.0) for each of the described regions above.

To finally obtain the relative coordinates of the hexagonal center associated with the identified region, in step 1523, the set of converted Boolean values may be used as an input to a single floating point vectorial function operable to map each set of these values to a set of xy coordinates of the associated element center. For example, in the described embodiment and as shown in FIG. 18C, one obtains the relative position vectors of each hexagonal center r with the vectorial function:

$$\vec{r} = ( {r}_{x} , {r}_{y} ) = ( 0.5 + 0.5 * ( c a s e R - c a s e L ) , \frac{2}{3} - ( c a s e R - c a s e L ) )$$

thus, the inputs of (1.0, 0.0), (0.0, 1.0) or (0.0, 0.0) map to the positions (0.0, −⅓), (0.5, ⅔), and (1.0, −⅓), respectively, which corresponds to the shown hexagonal centers 1863, 1865 and 1867 shown in FIG. 18C, respectively, in the rescaled coordinates.

Now back to FIG. 15, we may proceed with the final step 1525 to translate the relative coordinates obtained above to absolute 3D coordinates with respect to the display or similar (i.e. in mm). First, the coordinates of the hexagonal tile center and the coordinates of the bottom left corner are added to get the position of the hexagonal tile center in the optical layer's frame of reference. As needed, the process may then scale back the values into absolute units (i.e. mm) and rotate the coordinates back to the original frame of reference with respect to the display to obtain the 3D positions (in mm) of the optical layer element's center with respect to the display's frame of reference, which is then fed into step 1116.

The skilled artisan will note that modifications to the above-described method may also be used. For example, the staggered grid shown in FIG. 17A may be translated higher by a value of ⅓ (in normalized units) so that within each rectangle the diagonals separating each region are located on the upper left and right corners instead. The same general principles described above still applies in this case, and the skilled technician will understand the minimal changes to the equations given above will be needed to proceed in such a fashion. Furthermore, as noted above, different LFSL element geometries can result in the delineation of different (normalized) rectangular tile regions, and thus, the formation of corresponding conditional boundary statements and resulting binary/Boolean region-identifying and center-locating coordinate systems/functions.

In yet other embodiments, wherein a rectangular and/or square microlens array is used instead of a nestled (hexagonal) array, a slightly different method may be used to identify the associated LFSL element (microlens) center (step 1114). Herein, the microlens array is represented by an array of rectangular and/or square tiles. The method, as previously described, goes through step 1515, where the x and y coordinates are rescaled (normalized) with respect to a microlens x and y dimension (henceforth giving each rectangular and/or square tile a width and height of 1 unit). However, at step 1517, the floor( ) function is used directly on each x and y coordinates of \vec{uv} (the position vector of intersection point 1411) to find the coordinates of the bottom left corner associated with the corresponding square/rectangular tile. Therefrom, the relative coordinates of the tile center from the bottom left corner are added directly to obtain the final scaled position vector:

$$\vec{r} = ( {r}_{x} , {r}_{y} ) = ( floor ( {uv}_{x} ) + 0.5 , floor ( {uv}_{y} ) + 0.5 )$$

Once this vector is known, the method goes directly to step 1525 where the coordinates are scaled back into absolute units (i.e. mm) and rotated back to the original frame of reference with respect to the display to obtain the 3D positions (in mm) of the optical layer element's center with respect to the display's frame of reference, which is then fed into step 1116.

The light field rendering methods described above (from FIGS. 11 to 20D) may also be applied, in some embodiments, at a subpixel level in order to achieve an improved light field image resolution. Indeed, a single pixel on a color subpixelated display is typically made of several color primaries, typically three colored elements—ordered (on various displays) either as blue, green and red (BGR) or as red, green and blue (RGB). Some displays have more than three primaries such as the combination of red, green, blue and yellow (RGBY) or red, green, blue and white (RGBW), or even red, green, blue, yellow and cyan (RGBYC). Subpixel rendering operates by using the subpixels as approximately equal brightness pixels perceived by the luminance channel. This allows the subpixels to serve as sampled image reconstruction points as opposed to using the combined subpixels as part of a “true” pixel. For the light field rendering methods as described above, this means that the center position of a given pixel (e.g. pixel 1401 in FIG. 14) is replaced by the center positions of each of its subpixel elements. Therefore, the number of color samples to be extracted is multiplied by the number of subpixels per pixel in the digital display. The methods may then follow the same steps as described above and extract the associated image portions of each subpixel individually (sequentially or in parallel).

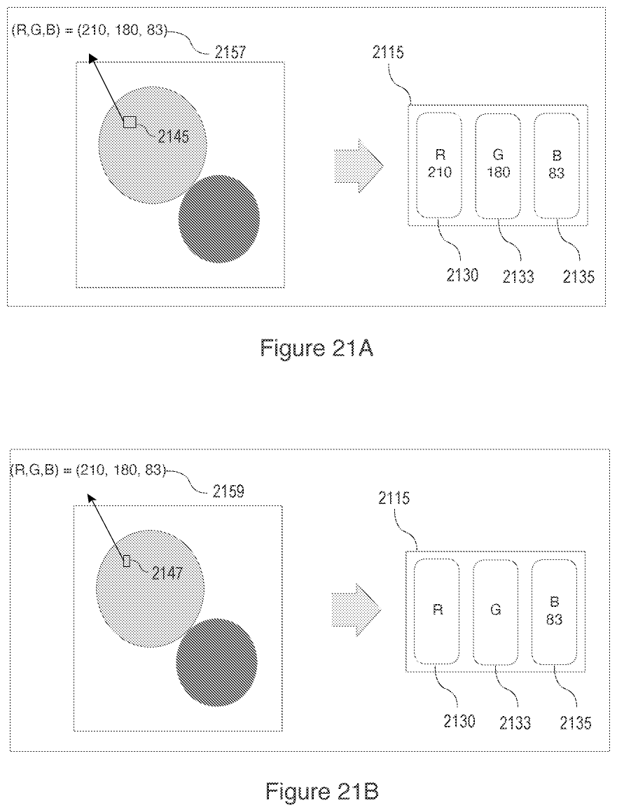

In FIG. 21A, an exemplary pixel 2115 is comprised of three RBG subpixels (2130 for red. 2133 for green and 2135 for blue). Other embodiments may deviate from this color partitioning, without limitation. When rendering per pixel, as described in FIG. 11 or in FIG. 19, the image portion 2145 associated with said pixel 2115 is sampled to extract the luminance value of each RGB color channels 2157, which are then all rendered by the pixel at the same time. In the case of subpixel rendering, as illustrated in FIG. 21B, the methods find the image portion 2147 associated with blue subpixel 2135. Therefore, only the subpixel channel intensity value of RGB color channels 2159 corresponding to the target subpixel 2135 is used when rendering (herein the blue subpixel color value, the other two values are discarded). In doing so, a higher adjusted image resolution may be achieved for instance, by adjusting adjusted image pixel colours on a subpixel basis, and also optionally discarding or reducing an impact of subpixels deemed not to intersect or to only marginally intersect with the user's pupil.

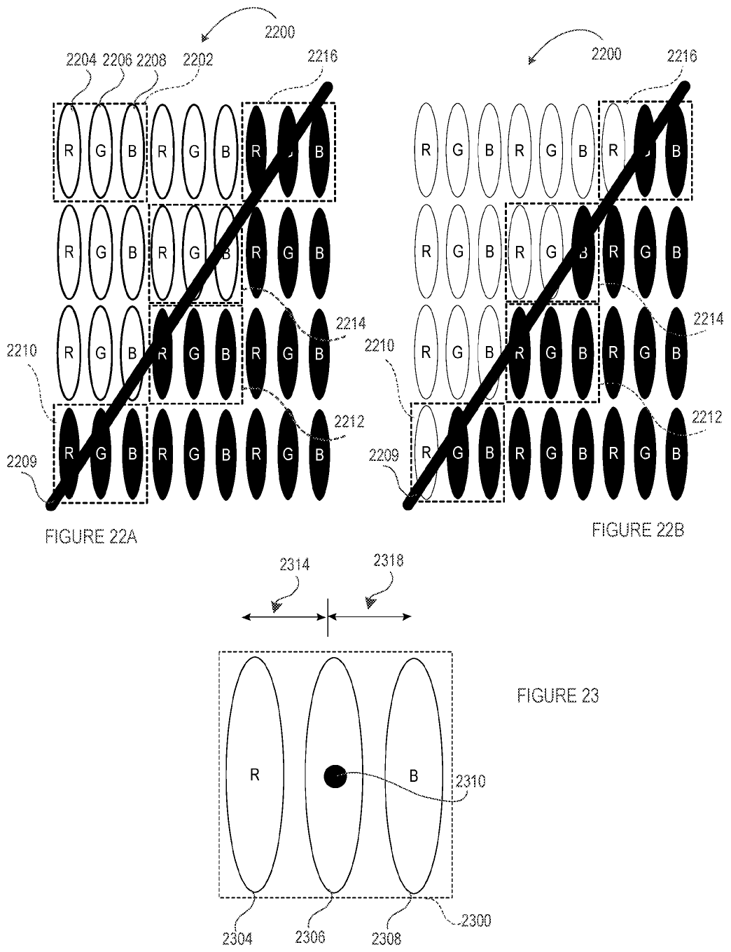

To further illustrate embodiments making use of subpixel rendering, with reference to FIGS. 22A and 22B, a (LCD) pixel array 2200 is schematically illustrated to be composed of an array of display pixels 2202 each comprising red (R) 2204, green (G) 2206, and blue (B) 2208 subpixels. As with the examples provided above, to produce a light field display, a light field shaping layer, such as a microlens array, is to be aligned to overlay these pixels such that a corresponding subset of these pixels can be used to predictably produce respective light field rays to be computed and adjusted in providing a corrected image. To do so, the light field ray ultimately produced by each pixel can be calculated knowing a location of the pixel (e.g. x,y coordinate on the screen), a location of a corresponding light field element through which light emanating from the pixel will travel to reach the user's eye(s), and optical characteristics of that light field element, for example. Based on those calculations, the image correction algorithm will compute which pixels to light and how, and output subpixel lighting parameters (e.g. R. G and B values) accordingly. As noted above, to reduce computation load, only those pixels producing rays that will interface with the user's eyes or pupils may be considered, for instance, using a complementary eye tracking engine and hardware, though other embodiments may nonetheless process all pixels to provide greater buffer zones and/or a better user experience.

In the example shown in FIG. 22A, an angular edge 2209 is being rendered that crosses the surfaces of affected pixels 2210, 2212, 2214 and 2216. Using standard pixel rendering, each affected pixel is either turned on or off, which to some extent dictates a relative smoothness of the angular edge 2209.

In the example shown in FIG. 22B, subpixel rendering is instead favoured, whereby the red subpixel in pixel 2210, the red and green subpixels in pixel 2214 and the red subpixel in pixel 2216 are deliberately set to zero (0) to produce a smoother representation of the angular edge 2209 at the expense of colour trueness along that edge, which will not be perceptible to the human eye given the scale at which these modifications are being applied. Accordingly, image correction can benefit from greater subpixel control while delivering sharper images.

In order to implement subpixel rendering in the context of light field image correction, in some embodiments, ray tracing calculations must be executed in respect of each subpixel, as opposed to in respect of each pixel as a whole, based on a location (x,y coordinates on the screen) of each subpixel. Beyond providing for greater rendering accuracy and sharpness, subpixel control and ray tracing computations may accommodate different subpixel configurations, for example, where subpixel mixing or overlap is invoked to increase a perceived resolution of a high resolution screen and/or where non-uniform subpixel arrangements are provided or relied upon in different digital display technologies.

In some embodiments, however, in order to avoid or reduce a computation load increase imparted by the distinct consideration of each subpixel, some computation efficiencies may be leveraged by taking into account the regular subpixel distribution from pixel to pixel, or in the context of subpixel sharing and/or overlap, for certain pixel groups, lines, columns, etc. With reference to FIG. 23, a given pixel 2300, much as those illustrated in FIGS. 22A and 22B, is shown to include horizontally distributed red (R) 2304, green (G) 2306, and blue (B) 2308 subpixels. Using standard pixel rendering and ray tracing, light emanating from this pixel can more or less be considered to emanate from a point located at the geometric center 2310 of the pixel 2300. To implement subpixel rendering, ray tracing could otherwise be calculated in triplicate by specifically addressing the geometric location of each subpixel. Knowing the distribution of subpixels within each pixel, however, calculations can be simplified by maintaining pixel-centered computations and applying appropriate offsets given known geometric subpixel offsets (i.e. negative horizontal offset 2314 for the red subpixel 2304, a zero offset for the green 2306 and a positive horizontal offset 2318 for the blue subpixel 2308). In doing so, light field image correction can still benefit from subpixel processing without significantly increased computation load.

While this example contemplates a linear (horizontal) subpixel distribution, other 2D distributions may also be considered without departing from the general scope and nature of the present disclosure. For example, for a given digital display screen and pixel and subpixel distribution, different subpixel mappings can be determined to define respective pixel subcoordinate systems that, when applied to standard pixel-centric ray tracing and image correction algorithms, can allow for subpixel processing and increase image correction resolution and sharpness without undue processing load increases.

In some embodiments, additional efficiencies may be leveraged on the GPU by storing the image data, for example image 1306, in the GPU's texture memory. Texture memory is cached on chip and in some situations is operable to provide higher effective bandwidth by reducing memory requests to off-chip DRAM. Specifically, texture caches are designed for graphics applications where memory access patterns exhibit a great deal of spatial locality, which is the case of the steps 1110-1126 of method 1100. For example, in method 1100, image 1306 may be stored inside the texture memory of the GPU, which then greatly improves the retrieval speed during step 1126 where the color channel associated with the portion of image 1306 at intersection point 1423 is determined.

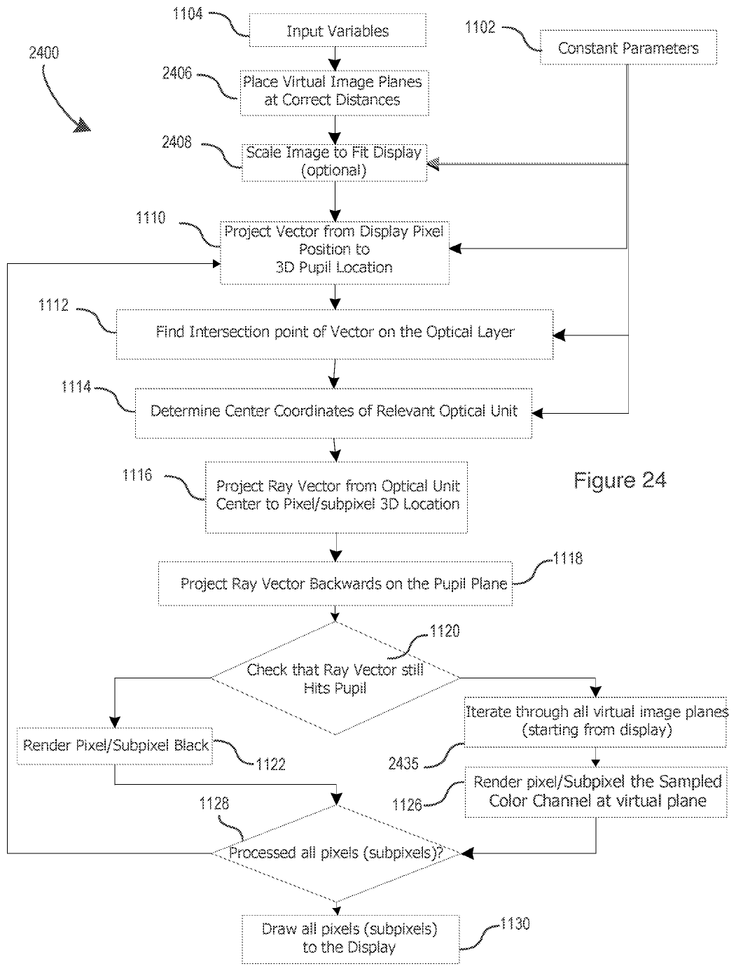

With reference to FIGS. 24 to 26D, and in accordance with one embodiment, an exemplary computationally implemented ray-tracing method for rendering multiple images or image portions on multiple adjusted distinct image planes simultaneously via an array of light field shaping elements, or light field shaping layer (LFSL) thereof, will now be described. The previous above-described embodiments were directed to correcting a single image by directly or indirectly modifying the location of the virtual image plane and/or eye focal plane. In contrast, the below-described embodiments are directed to a light field display which is generally operable to display multiple image planes at different locations/depths simultaneously. In some embodiments, distinct image planes may be juxtaposed such that different sides or quadrants of an image, for example, may be perceived at different depths. In such embodiments, a different effective vision correction parameter (e.g. diopter), or depth, may be applied, to each portion or quadrant. While this approach may result in some distortions or artefacts at the edges of the areas or quadrants, depending on the image date to be rendered along these edges, such artefacts may be negligible if at all perceivable. In other embodiments, however, different image portions may be at least partially superimposed such that portions at different depths, when viewed from particular perspectives, may indeed appear to overlap. This enables a user to focus on each plane individually, thus creating a 2.5D effect. Thus, a portion of an image may mask or obscure a portion of another image located behind it depending on the location of the user's pupil (e.g. on an image plane perceived to be located at an increased distance from the display than the one of the first image portion). Other effects may include parallax motion between each image plane when the user moves. The following provides a more detailed description of an embodiment in which overlapping portions may be addressed via an applicable transparency parameter resolved by processing each virtual image portion layer by layer.

Method 2400 of FIG. 24 substantially mirrors method 1100 of FIG. 11, but generalizes it to include multiple distinct virtual image planes. Thus, new steps 2406, 2408, and 2435 have been added, while steps 1110 to 1122, and 1126 to 1130 are the same as already described above. Meanwhile, when considering a fixed refractor installation, the input of constant parameters 1102 may, in such cases, be fixed and integrally designed within operation of the device/system.

For example, to account for multiple distinct image planes, image data 1306 of input variables 1104 may also include depth information. Thus, any image or image portion may have a respective depth indicator. Thus, at step 2406, a set of multiple virtual image planes may be defined. On these planes, images or image portions may be present. Areas around these images may be defined as transparent or see-through, meaning that a user would be able to view through that virtual image plane and see, for example, images or image portions located behind it. At step 2408, any image or image portion on these virtual image planes may be optionally scaled to fit the display.

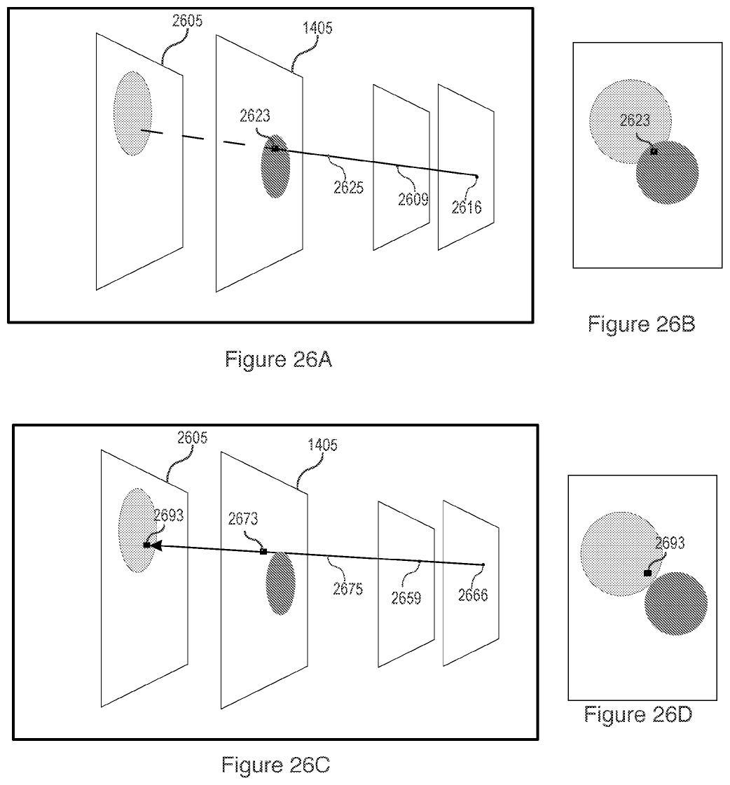

As an example, in the previous example of FIGS. 14A-14C, a single virtual image plane 1405, showing two circles, was shown. In contrast, FIGS. 26A and 26B show an example wherein each circle is located on its own image plane (e.g. original virtual plane 1405 with new virtual image plane 2605). The skilled technician will understand that two planes are shown only as an example and that the method described herein applies equally well to any number of virtual planes. The only effect of having more planes is a larger computational load.

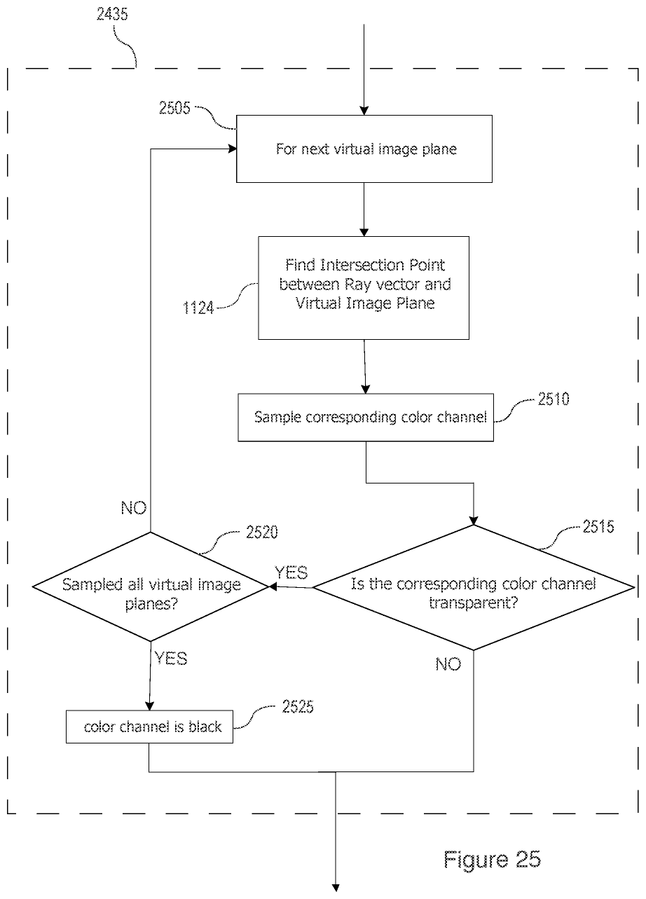

Going back to FIG. 24, steps 1110 to 1122 occur similarly to the ones described in FIG. 11. However, step 1124 has been included and expanded upon in Step 2435, which is described in FIG. 25. In step 2435, an iteration is done over the set of virtual image planes to compute which image portion from which virtual image plane is seen by the user. Thus, at step 2505 a virtual image plane is selected, starting from the plane located closest to the user. Then step 1124 proceeds as described previously for that selected virtual plane. At step 2510 the corresponding color channel of the intersection point identified at step 1124 is sampled. Then at step 2515, a check is made to see if the color channel is transparent. If this is not the case, then the sampled color channel is sent to step 1126 of FIG. 24, which was already described and where the color channel is rendered by the pixel/subpixel. An example of this is illustrated in FIGS. 26A and 26B, wherein a user is located so that a ray vector 2625 computed passing through optical element 2616 and pixel/subpixel 2609 intersects virtual image plane 1405 at location 2623. Since this location is non-transparent, this is the color channel that will be assigned to the pixel/subpixel. However, as this example shows, this masks or hides parts of the image located on virtual image plane 2605. Thus, an example of the image perceived by the user is shown in FIG. 26B.

Going back to FIG. 25, at step 2515 if the color channel is transparent, then another check is made at step 2520 to see if all virtual image planes have been iterated upon. If this is the case, then that means that no image or image portion is seen by the user and at step 2525, for example, the color channel is set to black (or any other background colour), before proceeding to step 1126. If however at least one more virtual image plane is present, then the method goes back to step 2505 and selects that next virtual image plane and repeats steps 1124, 2510 and 2515. An example of this is illustrated in FIG. 26C, wherein a user is located so that a distinct ray vector 2675 computed passing through optical element 2666 and pixel/subpixel 2659 first intersects at location 2673 of virtual image plane 1405. This location is defined to be transparent, so the method checks for additional virtual image planes (here plane 2605) and computes the intersection point 2693, which is non-transparent, and thus the corresponding color channel is selected. An example of the image perceived by the user is shown in FIG. 26D.

Going back to FIG. 24, once the pixel/subpixel has been assigned the correct color channel at step 1126, the method proceeds as described previously at steps 1128 and 1130.

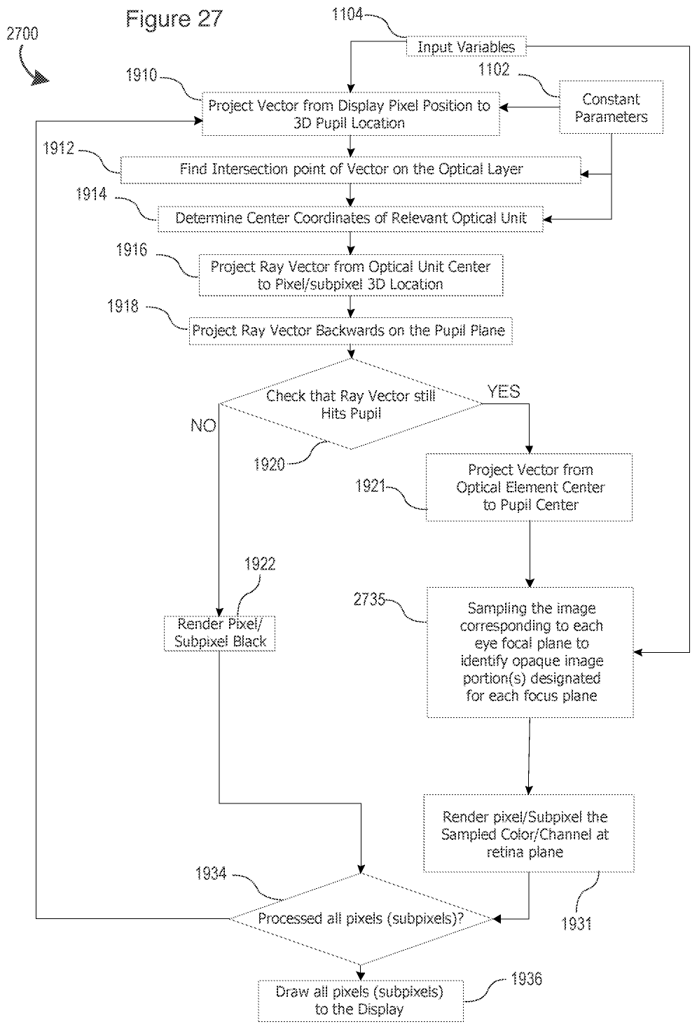

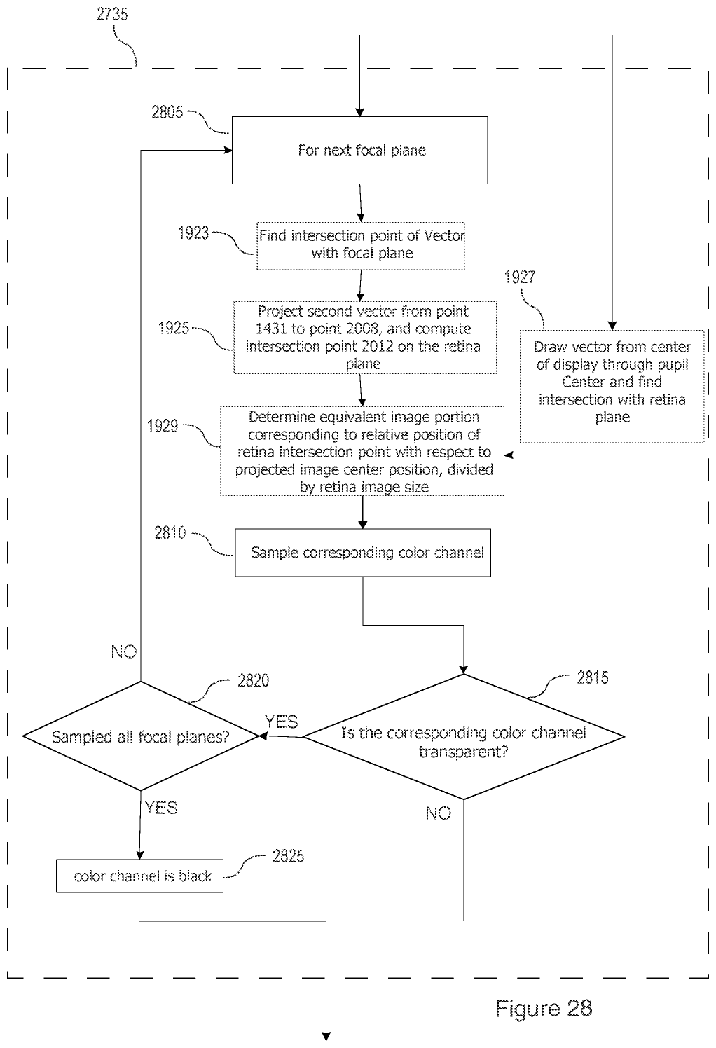

Similarly, method 2700 of FIG. 27 substantially mirrors method 1900 of FIG. 19 but also generalizes it to include multiple distinct eye focal planes (each corresponding with a virtual image plane, including infinity, as explained above). Thus, in method 2700, steps 1910 to 1921 and 1931 to 1936 are the same as described for method 1900. The difference comes from new step 2735 which includes and expands upon steps 1921 to 1929, as shown in FIG. 28. There, we see that the method iterates over all designated image planes, each corresponding with a different eye focal plane, starting from the plane corresponding to an image located closest to the user. Thus, a new eye focal plane is selected at step 2805, which is used for steps 1923 to 1929 already described above. Once the corresponding image portion is located at step 1929, at step 2810, the corresponding pixel/subpixel color channel is sampled. Then at step 2815, if the color channel is non-transparent, then the method goes back to step 1931 of FIG. 27, wherein the pixel/subpixel is assigned that color channel. However, if the image portion is transparent, then the method iterates to the eye focal plane corresponding to the next designated image plane. Before this is done, the method checks at step 2820 if all the eye focal planes have been iterated upon. If this is the case, then no image portion will be selected and at step 2825 the color channel is set to black, for example, before exiting to step 1931. If other eye focal planes are still available, then the method goes back to step 2805 to select the next eye focal plane and the method iterates once more.

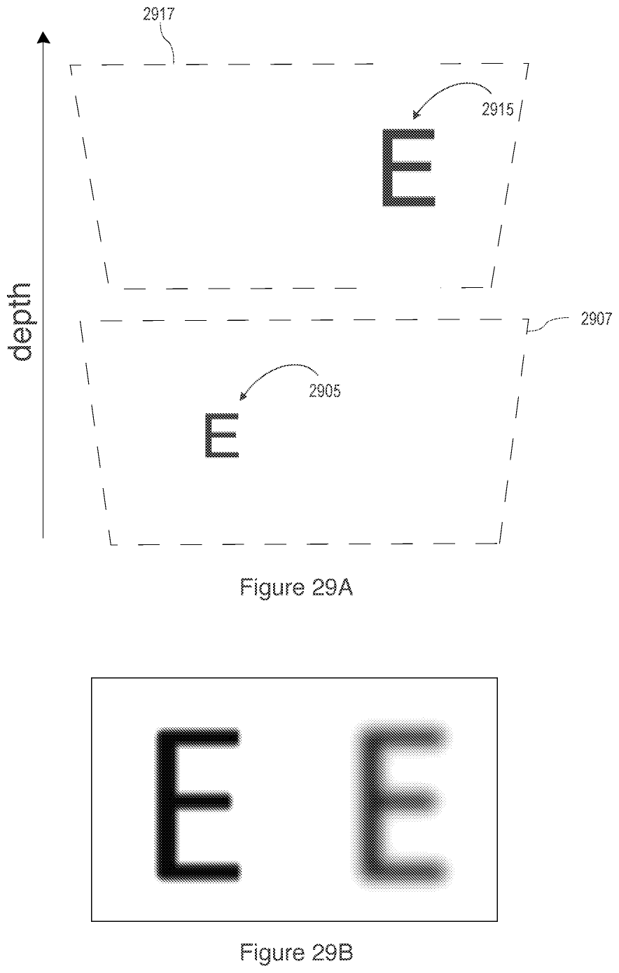

In some embodiments, methods 2400 or 2700 may be used to implement a phoropter/refractor device to do subjective visual acuity evaluations. For example, as illustrated in FIGS. 29A and 29B, different optotypes (e.g. letters, symbols, etc.) may be displayed simultaneously but at different perceived depths, to simulate the effect of adding a refractive optical component (e.g. change in focus/optical power). In FIG. 29A, two images of the same optotype (e.g. letter E) are displayed, each on their own designated image plane (e.g. here illustrated as virtual image planes as an example only). In this example, image 2905 is located on designated image plane 2907 while image 2915 is located on designated image plane 2917, which is located further away. Optionally, as illustrated herein, the size of the image may be increased with increased depth so that all images displayed are perceived to be of a similar relative size by the user. In FIG. 29B, we see an example of the perception of both images as perceived by a user with reduced visual acuity (e.g. myopia), for example, wherein the image closest to the user is seen to be clearer. Thus, a user could be presented with multiple images (e.g. 2 side-by-side, 4, 6 or 9 in a square array, etc.) and indicate which image is clearer and/or most comfortable to view. An eye prescription may then be derived from this information. Moreover, in general, both spherical and cylindrical power may be induced by the light field display.

Accordingly, it can be observed that the ray-tracing methods 2400 and 2700 noted above, and related light field display solutions, can be equally applied to image perception adjustment solutions for visual media consumption, as they can for subjective vision testing solutions, or other technologically related fields of endeavour. As alluded to above, the light field display and rendering/ray-tracing methods discussed above may all be used to implement, according to various embodiments, a subjective vision testing device or system such as a phoropter or refractor. Indeed, a light field display may replace, at least in part, the various refractive optical components usually present in such a device. Thus, the vision correction light field ray tracing methods 1100, 1900, 2400, or 2700 discussed above may equally be applied to render optotypes at different dioptric power or refractive correction by generating vision correction for hyperopia (far-sightedness) and myopia (nearsightedness), as was described above in the general case of a vision correction display. Light field systems and methods described herein, according to some embodiments, may be applied to create the same capabilities as a traditional instrument and to open a spectrum of new features, all while improving upon many other operating aspects of the device. For example, the digital nature of the light field display enables continuous changes in dioptric power compared to the discrete change caused by switching or changing a lens or similar; displaying two or more different dioptric corrections seamlessly at the same time; and, in some embodiments, the possibility of measuring higher-order aberrations and/or to simulate them for different purposes such as, deciding for free-form lenses, cataract surgery operation protocols, IOL choice, etc.

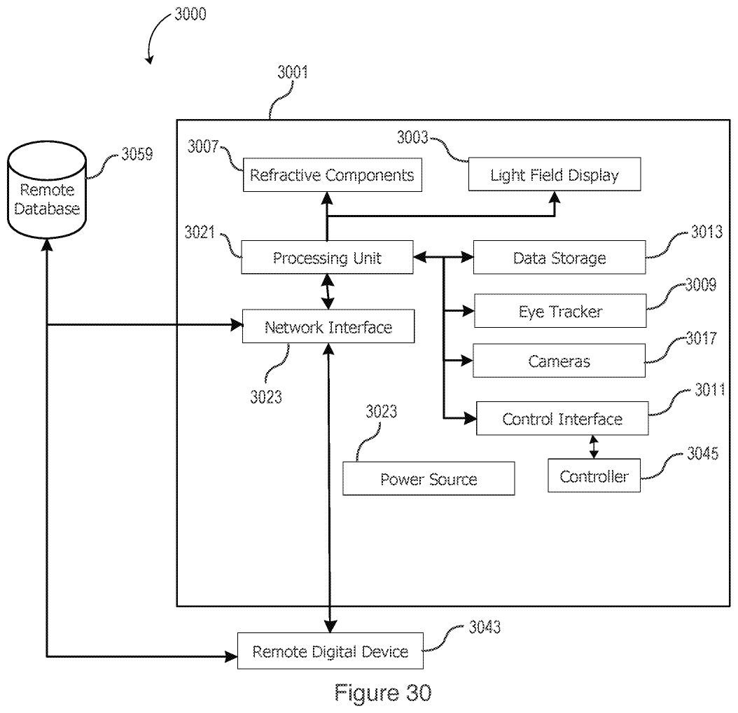

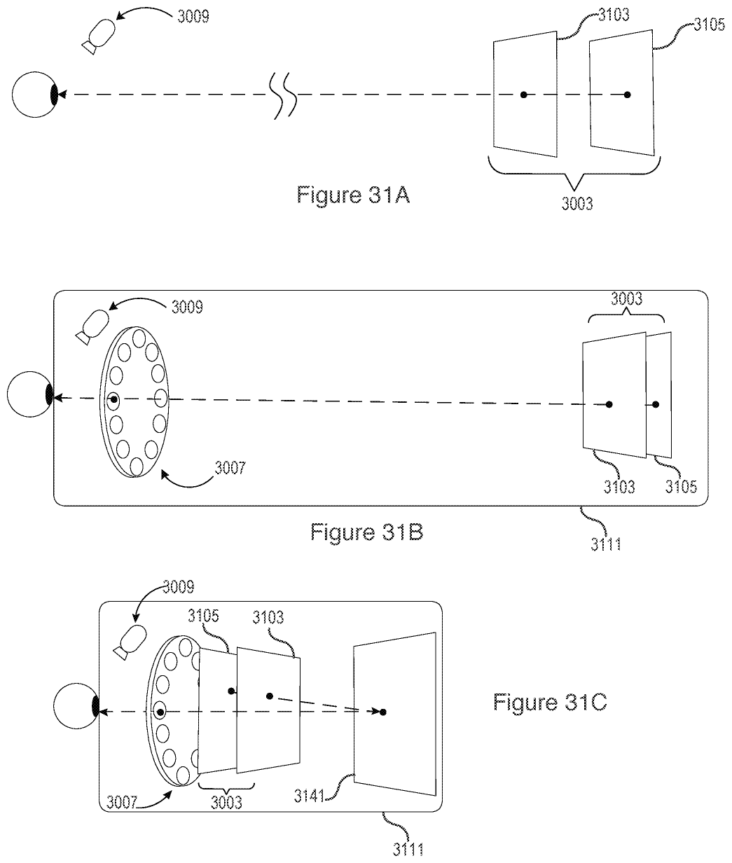

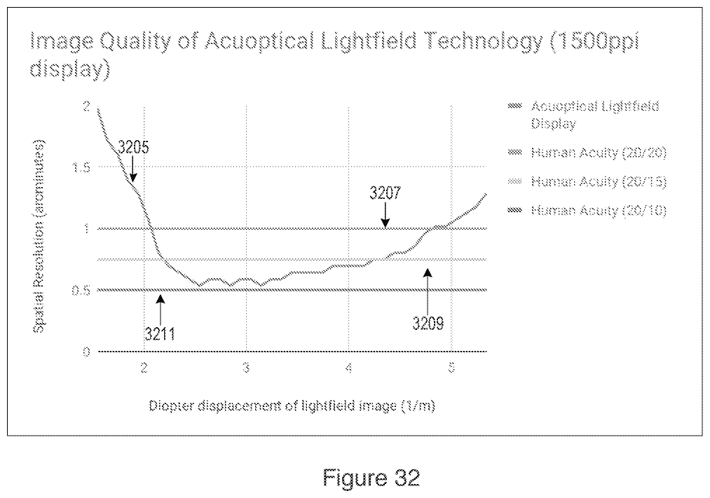

With reference to FIGS. 30, and 31A to 31C, and in accordance with different embodiments, an exemplary subjective vision testing system, generally referred to using the numeral 3000, will now be described. At the heart of this system is a light field vision testing device such as a light field refractor or phoropter 3001. Generally, the light field phoropter 3001 is a device comprising, at least in part, a light field display 3003 and which is operable to display or generate one or more optotypes to a patient having his/her vision acuity (e.g. refractive error) tested. In some embodiments, the light field phoropter may comprise an eye tracker 3009 (such as a near-IR camera or other as discussed above) that may be used to determine the pupil center position in real-time or near real-time, for accurately locating the patient's pupil, as explained above with regard to the ray-tracing methods 1100, 1900, 2400, or 2700. Indeed, FIG. 32 shows a plot of the angular resolution (in arcminutes) of an exemplary light field display comprising a 1500 ppi digital pixel display as a function of the dioptric power of the light field image (in diopters). We clearly see that, in this particular example, the light field display is able to generate displacements (line 3205) in diopters that have higher resolution corresponding to 20/20 vision (line 3207) or better (e.g. 20/15-line 3209) and close to (20/10—line 3211)), here within a dioptric power range of 2 to 2.5 diopters. Thus, the light field displays and ray-tracing methods described above, according to different embodiments, may be used to replace, at least in part, traditional optical components. In some embodiments, a head-rest, eyepiece or similar (not shown) may be used to keep the patient's head still and in the same location, thus in such examples, foregoing the general utility of a pupil tracker or similar techniques by substantially fixing a pupil location relative to this headrest. In some embodiments, phoropter 3001 may comprise a network interface 3023 for communicating via network to a remote database or server 3059.

For example, in one embodiment and as illustrated in FIG. 31A, the light field phoropter 3001 may comprise light field display 3003 (herein comprising a MLA 3103 and a digital pixel display 3105) located relatively far away (e.g. one or more meters) from the patient′ eye currently being diagnosed. Note that the pointed line is used to schematically illustrate the direction of the light rays emitted by the display 3105. Also illustrated is the eye-tracker 3009, which may be provided as a physically separate element, for example, installed in at a given location in a room or similar. In some embodiments, the noted eye/pupil tracker may include the projection of IR markers/patterns to help align the patient's eye with the light field display. In some embodiments, a tolerance window (e.g. “eye box”) may be considered to limit the need to refresh the ray-tracing iteration. An exemplary value of the size of the eye box, in some embodiments, is around 6 mm, though smaller (e.g. 4 mm) or larger eye boxes may alternatively be set to impact image quality, stability or like operational parameters.

Going back to FIG. 30, light field phoropter 3001 may also comprise, according to different embodiments and as will be further discussed below, one or more refractive optical components 3007, a processing unit 3021, a data storage unit or internal memory 3013, a network interface 3023, one or more cameras 3017 and a power module 3023.

In some embodiments, power module 3023 may comprise, for example, a rechargeable Li-ion battery or similar. In some embodiments, it may comprise an additional external power source, such as, for example, a USB-C external power supply. It may also comprise a visual indicator (screen or display) for communicating the device's power status, for example whether the device is on/off or recharging.

In some embodiments, internal memory 3013 may be any form of electronic storage, including a disk drive, optical drive, read-only memory, random-access memory, or flash memory, to name a few examples. In some embodiments, a library of chart patterns (Snellen charts, prescribed optotypes, forms, patterns, or other) may be located in internal memory 3013 and/or retrievable from remote server 3059.

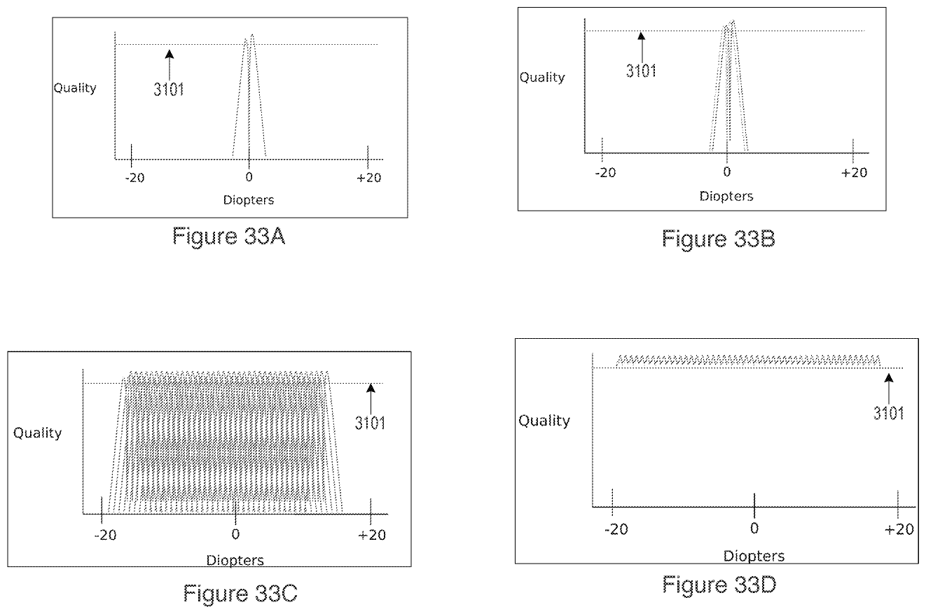

In some embodiments, one or more optical components 3007 can be used in combination with the light field display 3003, for example to shorten the device's dimensions and still offer an acceptable range in dioptric power. The general principle is schematically illustrated in the plots of FIGS. 33A to 33D. In these plots, the image quality (e.g. inverse of the angular resolution, higher is better) at which optotypes are small enough to be useful for vision testing in this plot is above horizontal line 3101 which represents typical 20/20 vision. FIG. 33A shows the plot for the light field display only, where we see the characteristic two peaks corresponding to the smallest resolvable point, one of which was plotted in FIG. 32 (here inverted and shown as a peak instead of a basin), and where each region above the line may cover a few diopters of dioptric power, according to some embodiments. While the dioptric range may, in some embodiments, be more limited than needed when relying only on the light field display, it is possible to shift this interval by adding one or more refractive optical components. This is shown in FIG. 33B where the regions above the line 3101 is shifted to the left (negative diopters) by adding a single lens in the optical path.

Thus, by using a multiplicity of refractive optical components or by alternating sequentially between different refractive components of increasing or decreasing dioptric power, it is possible to shift the center of the light field diopter range to any required value, as shown in FIG. 33C, and thus the image quality may be kept above line 3101 for any required dioptric power as shown in FIG. 33D. In some embodiments, a range of 30 diopters from +10 to −20 may be covered for example. In the case of one or more reels of lenses, the lens may be switched for a given larger dioptric power increment, and the light field display would be used to provide a finer continuous change to accurately pin-point the required total dioptric power required to compensate for the patient's reduced visual acuity. This would still result in light field phoropter 3001 having a reduced number of refractive optical components compared to the number of components needed in a traditional phoropter, while drastically enhancing the overall fine-tuning ability of the device.

One example, according to one embodiment, of such a light field phoropter 3001 is schematically illustrated in FIG. 31B, wherein the light field display 3003 (herein shown again comprising MLA 3103 and digital pixel display 3105) is combined with a multiplicity of refractive components 3007 (herein illustrate as a reel of lenses as an example only). By changing the refractive component used in combination with the light field display, a larger dioptric range may be covered. This may also provide means to reduce the device's dimension, making it in some embodiments more portable, and encompass all its internal components into a shell, housing or casing 3111. In some embodiments, the light field phoropter may comprise a durable ABS housing and may be shock and harsh-environment resistant. In some embodiments, the light field phoropter 3001 may comprise a telescopic feel for fixed or portable usage; optional mounting brackets, and/or a carrying case. In some embodiments, all components may be internally protected and sealed from the elements.

In some embodiments, the casing may further comprise an eye piece or similar that the patient has to look through, which may limit movement of the patient's eye during diagnostic and/or indirectly provide a pupil location to the light field renderer.

In some embodiments, it may also be possible to further reduce the size of the device by adding, for example, a mirror or any device which may increase the optical path. This is illustrated in FIG. 31C where the length of the device was reduced by adding a mirror 3141. This is shown schematically by the pointed arrow which illustrates the light being emitted from pixel display 3105 travelling through MLA 3103 before being reflected by mirror 3141 back through refractive components 3007 and ultimately hitting the eye.

The skilled technician will understand that different examples of refractive components 3007 may include, without limitation, one or more lenses, sometimes arranged in order of increasing dioptric power in one or more reels of lenses similar to what is typically found in traditional phoropters; an electrically controlled fluid lens; active Fresnel lens; and/or Spatial Light Modulators (SLM). In some embodiments, additional motors and/or actuators may be used to operate refractive components 3007. These may be communicatively linked to processing unit 3021 and power module 3023, and operate seamlessly with light display 3003 to provide the required dioptric power.

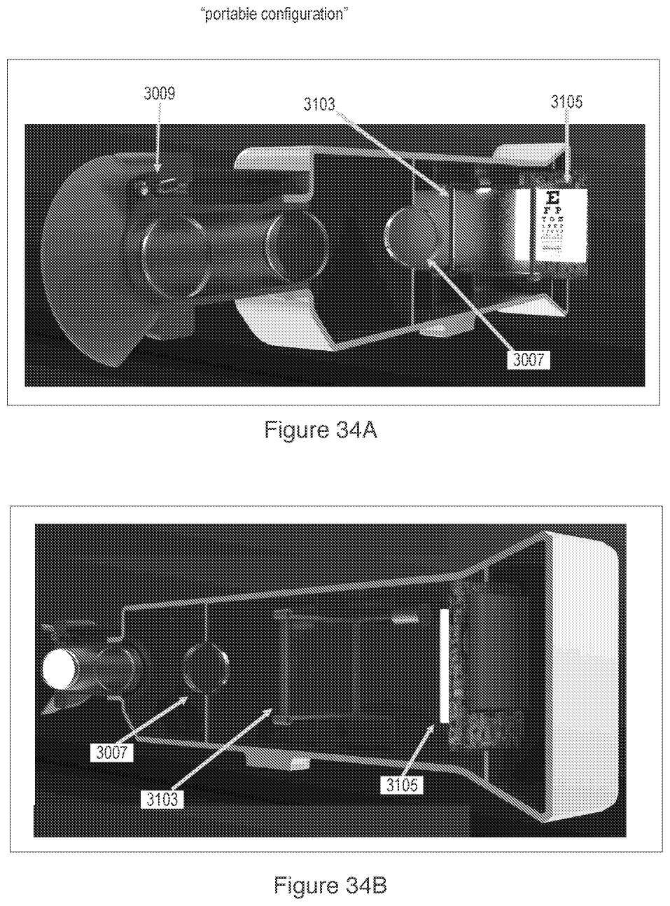

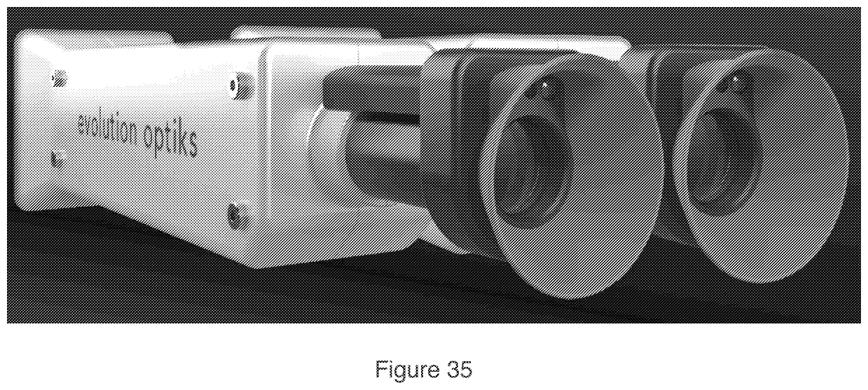

For example, FIGS. 34A and 34B show a perspective view of an exemplary light field phoropter 3001 similar to the one of FIG. 31B, but wherein the refractive component 3007 is an electrically tunable liquid lens. Thus, in this particular embodiment, no mechanical or moving component are used, which may result in the device being more robust. In some embodiments, the electrically tunable lens may have a range of +13 diopters.

In one illustrative embodiment, a 1000 dpi display is used with a MLA having a 65 mm focal distance and 1000 μm pitch with the user's eye located at a distance of about 26 cm. A similar embodiment uses the same MLA and user distance with a 3000 dpi display.

Other displays having resolutions including 750 dpi, 1000 dpi, 1500 dpi and 3000 dpi were also tested or used, as were MLAs with a focal distance and pitch of 65 mm and 1000 μm, 43 mm and 525 μm, 65 mm and 590 μm, 60 mm and 425 μm, 30 mm and 220 μm, and 60 mm and 425 μm, respectively, and user distances of 26 cm, 45 cm or 65 cm.

Going back to FIG. 30, in some embodiments, eye-tracker 3009 may be a digital camera, in which case it may be used to further acquire images of the patient's eye to provide further diagnostics, such as pupillary reflexes and responses during testing for example. In other embodiments, one or more additional cameras 3017 may be used to acquire these images instead. In some embodiments, light field phoropter 3001 may comprise built-in stereoscopic tracking cameras.

In some embodiments, feedback and/or control of the vision test being administered may be given via a control interface 3011. In some embodiments, the control interface 3011 may comprise a dedicated handheld controller-like device 3045. This controller 3045 may be connected via a cable or wirelessly, and may be used by the patient directly and/or by an operator like an eye professional. In some embodiments, both the patient and operator may have their own dedicated controller. In some embodiments, the controller may comprise digital buttons, analog thumbstick, dials, touch screens, and/or triggers.

In some embodiments, control interface 3011 may comprise a digital screen or touch screen, either on the phoropter device itself or on an external module. In other embodiments, the control interface may let other remote devices control the light field phoropter via the network interface. For example, remote digital device 3043 may be connected to light field phoropter by a cable (e.g. USB cable, etc.) or wirelessly (e.g. via Bluetooth or similar) and interface with the light field phoropter via a dedicated application, software or website. Such a dedicated application may comprise a graphical user interface (GUI), and may also be communicatively linked to remote database 3059.

In some embodiments, the patient may give feedback verbally and the operator may control the vision test as a function of that verbal feedback. In some embodiments, phoropter 3001 may comprise a microphone to record the patient's verbal communications, either to communicate them to a remote operator via network interface 3023 or to directly interact with the device (e.g. via speech recognition or similar).

In some embodiments, processing unit 3021 may be communicatively connected to data storage 3013, eye tracker 3009, light field display 3003 and refractive components 3007. Processing unit 3021 may be responsible for rendering one or more optotypes via light field display 3003 and, in some embodiments, jointly control refractive components 3007 to achieve a required total dioptric power. It may also be operable to send and receive data to internal memory 3013 or to/from remote database 3059.

In some embodiments, diagnostic data may be automatically transmitted/communicated to remote database 3059 or remote digital device 3043 via network interface 3023 through the use of a wired or wireless network connection. The skilled artisan will understand that different means of connecting electronic devices may be considered herein, such as, but not limited to, Wi-Fi, Bluetooth, NFC, Cellular, 2G, 3G, 4G, 5G or similar. In some embodiments, the connection may be made via a connector cable (e.g. USB including microUSB, USB-C, Lightning connector, etc.). In some embodiments, remote digital device 3043 may be located in a different room, building or city.

In some embodiments, two light field phoropters 3001 may be combined side-by-side to independently measure the visual acuity of both left and right eye at the same time. An example is shown in FIG. 35, where two units corresponding to the embodiment of FIGS. 34A and 34B (used as an example only) are placed side-by-side or fused into a single device.

In some embodiments, a dedicated application, software or website may provide integration with third party patient data software. In some embodiments, the phoropter's software may be updated on-the-fly via a network connection and/or be integrated with the patient's smartphone app for updates and reminders.

In some embodiments, the dedicated application, software or website may further provide a remote, real-time collaboration platform between the eye professional and patient, and/or between different eye professionals. This may include interaction between different participants via video chat, audio chat, text messages, etc.

In some embodiments, light field phoropter 3001 may be self-operated or operated by an optometrist, ophthalmologist or other certified eye-care professional. For example, in some embodiments, a patient could use phoropter 3001 in the comfort of his/her own home.

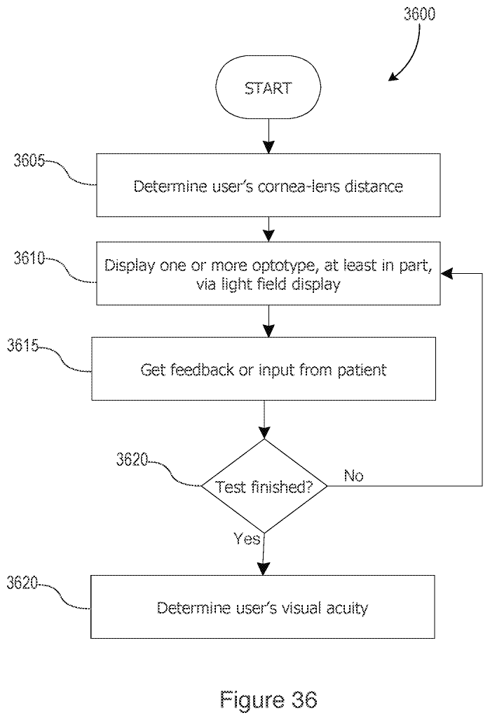

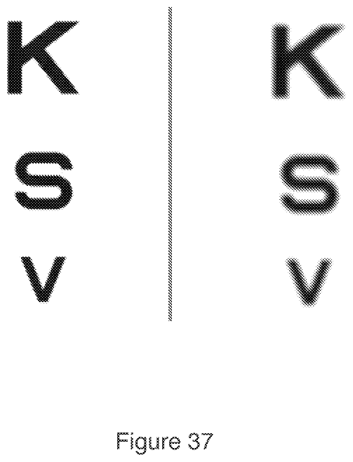

With reference to FIG. 36 and in accordance with different exemplary embodiments, a dynamic subjective vision testing method using vision testing system 3000, generally referred to using the numeral 3600, will now be described. As mentioned above, the use of a light field display enables phoropter 3001 of vision testing system 3000 to provide more dynamic and/or more modular vision tests than what is generally possible with traditional phoropters. Generally, method 3600 seeks to diagnose a patient's reduced visual acuity and produce therefrom, in some embodiments, an eye prescription or similar.

In some embodiments, eye prescription information may include, for each eye, one or more of: distant spherical, cylindrical and/or axis values, and/or a near (spherical) addition value.

In some embodiments, the eye prescription information may also include the date of the eye exam and the name of the eye professional that performed the eye exam. In some embodiments, the eye prescription information may also comprise a set of vision correction parameter(s) 201 used to operate any vision correction light field displays using the systems and methods described above. In some embodiments, the eye prescription may be tied to a patient profile or similar, which may contain additional patient information such as a name, address or similar. The patient profile may also contain additional medical information about the user. All information or data (i.e. set of vision correction parameter(s) 201, user profile data, etc.) may be kept on remote database 3059. Similarly, in some embodiments, the user's current vision correction parameter(s) may be actively stored and accessed from external database 3059 operated within the context of a server-based vision correction subscription system or the like, and/or unlocked for local access via the client application post user authentication with the server-based system.

Phoropter 3001 being, in some embodiments, portable, a large range of environment may be chosen to deliver the vision test (home, eye practitioner's office, etc.). At the start, the patient's eye may be placed at the required location. This is usually by placing his/her head on a headrest or by placing the objective (eyepiece) on the eye to be diagnosed. As mentioned above, the vision test may be self-administered or partially self-administered by the patient. For example, the operator (e.g. eye professional or other) may have control over the type of test being delivered, and/or be the person who generates or helps generate therefrom an eye prescription, while the patient may enter inputs dynamically during the test (e.g. by choosing or selecting an optotype, etc.).

As discussed above, the light field rendering method 3600 generally requires an accurate location of the patient's pupil center. Thus, at step 3605, such a location is acquired. In some embodiments, such a pupil location may be acquired via eye tracker 3009, either once, at intervals, or continuously. In other embodiments, the location may be derived from the device or system's dimension. For example, in some embodiments, the use an eye-piece or similar provides an indirect means of deriving the pupil location. In some embodiments, the phoropter 3001 may be self-calibrating and not require any additional external configuration or manipulation from the patient or the practitioner before being operable to start a vision test.

At step 3610, one or more optotypes is/are displayed to the patient, at one or more dioptric power (e.g. in sequence, side-by-side, or in a grid pattern/layout). The use of light field display 3003 offers multiple possibilities regarding how the optotypes are presented, and at which dioptric power each may be rendered. The optotypes may be presented sequentially at different dioptric power, via one or more dioptric power increments. In some embodiments, the patient and/or operator may control the speed and size of the dioptric power increments.

In some embodiments, optotypes may also be presented, at least in part, simultaneously on the same image but rendered at a different dioptric power (via ray-tracing methods 2400, or 2700, for example). For example, FIG. 37 shows an example of how different optotypes may be displayed to the patient but rendered with different dioptric power simultaneously. These may be arranged in columns or in a table or similar. In FIG. 37, we see two columns of three optotypes (K, S, V), varying in size, as they are perceived by a patient, each column being rendered at different degrees of refractive correction (e.g. dioptric power). In this specific example, the optotypes on the right are being perceived as blurrier than the optotypes on the left.

Thus, at step 3615, the patient would communicate/verbalize this information to the operator or input/select via control interface 3011 the left column as the one being clearer. Thus, in some embodiments, method 3600 may be configured to implement dynamic testing functions that dynamically adjust one or more displayed optotype's dioptric power in real-time in response to a designated input, herein shown by the arrow going back from step 3620 to step 3610. In the case of sequentially presented optotypes, the patient may indicate when the optotypes shown are clearer. In some embodiments, the patient may control the sequence of optotypes shown (going back and forth as needed in dioptric power), and the speed and increment at which these are presented, until he/she identifies the clearest optotype. In some embodiments, the patient may indicate which optotype or which group of optotypes is the clearest by moving an indicator icon or similar within the displayed image.

In some embodiments, the optotypes may be presented via a video feed or similar.

In some embodiments, when using a reel of lenses or similar, discontinuous changes in dioptric power may be unavoidable. For example, the reel of lenses may be used to provide a larger increment in dioptric power, as discussed above. Thus, step 3610 may in this case comprise first displaying larger increments of dioptric power by changing lens as needed, and when the clearest or less blurry optotypes are identified, fine-tuning with continuous or smaller increments in dioptric power using the light field display. In the case of optotypes presented simultaneously, the refractive components 3007 may act on all optotypes at the same time, and the change in dioptric power between them may be controlled only by the light display 3003. In some embodiments, for example when using an electrically tunable fluid lens or similar, the change in dioptric power may be continuous.

In some embodiments, eye images may be recorded during steps 3610 to 3620 and analyzed to provide further diagnostics. For example, eye images may be compared to a bank or database of proprietary eye exam images and analyzed, for example via an artificial intelligence (AI) or Machine-learning (ML) system or similar. This analysis may be done by phoropter 3001 locally or via a remote server or database 3059.

Once the correct dioptric power needed to correct for the patient's reduced visual acuity is defined at step 3625, an eye prescription or vision correction parameter(s) may be derived from the total dioptric power used to display the best perceived optotypes.

In some embodiments, the patient, an optometrist or other eye-care professional may be able to transfer the patient's eye prescription directly and securely to his/her user profile store on said server or database 3059. This may be done via a secure website, for example, so that the new prescription information is automatically uploaded to the secure user profile on remote database 3059. In some embodiments, the eye prescription may be sent remotely to a lens specialist or similar to have prescription glasses prepared.

In some embodiments, the vision testing system 3000 may also or alternatively be used to simulate compensation for higher-order aberrations. Indeed, the light field rendering methods 1100, 1900, 2400, or 2700 described above may be used to compensation for higher order aberrations (HOA), and thus be used to validate externally measured or tested HOA via method 3600, in that a measured, estimated or predicted HOA can be dynamically compensated for using the system described herein and thus subjectively visually validated by the viewer in confirming whether the applied HOA correction satisfactory addresses otherwise experienced vision deficiencies. In one such embodiment, a HOA correction preview can be rendered, for example, in enabling users to appreciate the impact HOA correction (e.g. HOA compensating eyewear or contact lenses, intraocular lenses (IOL), surgical procedures, etc.), or different levels or precisions thereof, could have on their visual acuity. Alternatively, HOA corrections once validated can be applied on demand to provide enhanced vision correction capabilities to consumer displays.

Higher-order aberrations can be defined in terms of Zernike polynomials, and their associated coefficients. In some embodiments, the light field phoropter may be operable to help validate or confirm measured higher-order aberrations, or again to provide a preview of how certain HOA corrections may lead to different degrees of improved vision. To do so, in some embodiments, the ray-tracing methods 1100, 1900, 2400, or 2700 may be modified to account for the wavefront distortion causing the HOA which are characterized by a given set of values of the Zernike coefficients. Such an approach may include, in some embodiments, extracting or deriving a set of light rays corresponding to a given wavefront geometry. Thus, the light field display may be operable to compensate for the distortion by generating an image corresponding to an “opposite” wavefront aberration. In some embodiments, the corresponding total aberration values may be normalized for a given pupil size of circular shape. Moreover, in some embodiments, the wavefront may be scaled, rotated and transformed to account for the size and shape of the view zones. This may include concentric scaling, translation of pupil center, and rotation of the pupil, for example.

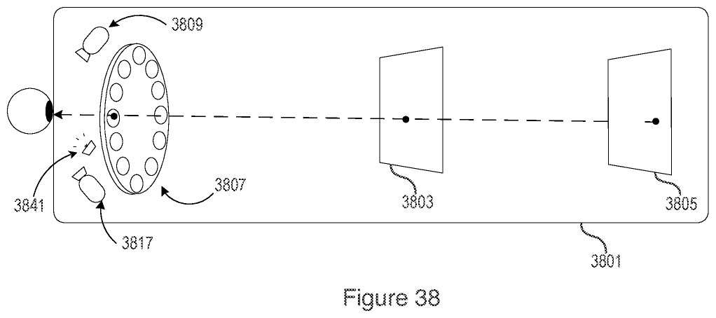

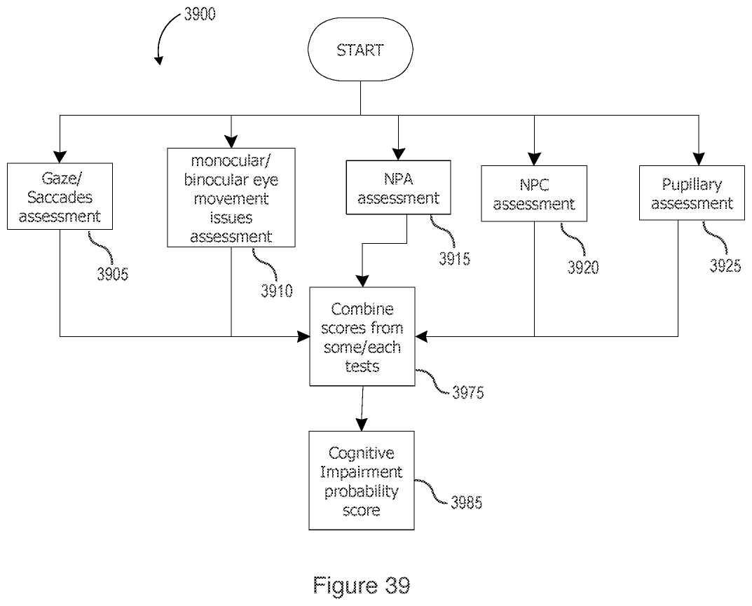

With reference to FIGS. 38 and 39 and in accordance with different exemplary embodiments, a cognitive impairment detection or testing method, generally referred to using with the numeral 3900, will now be described. In some embodiments, refractor 3001 described above, or a similar device, may be used to detect cognitive impairment in a patient. Indeed, cognitive impairment (e.g. caused for example by concussion) may be detectable by assessing the visual system of a patient. For example, in some cases mild traumatic brain injury (i.e. concussion) may cause common visual disorders like convergence insufficiency (CI), accommodative insufficiency (AI), and mild saccadic dysfunction (SD) to name just a few. Thus, a refractor as described herein may be leveraged via its light field imaging modalities to diagnose mild concussions or similar events causing cognitive impairment. An exemplary embodiment of a refractor 3801 configured for cognitive impairment detection is shown in the schematic diagram of FIG. 38, again illustratively comprising a MLA 3803 and a digital pixel display 3805. This schematic diagram is adapted from FIG. 31B and shows explicitly one or more cameras 3817 already discussed above and a one or more light sources 3841. Indeed, in some embodiments, some of these tests or assessments discussed below may require in some cases that an image or video of the user's eye to be recorded/acquired. This may be done for example using one or more cameras 3817 as discussed above, which may double as or for eye or pupil tracking system 3809. In some embodiments, machine vision methods may be used to facilitate the extraction of different features in real-time, for example and without limitation pupil size and/or saccade frequency or occurrences, etc. As shown in FIG. 38, in some embodiments, refractor 3801 may comprise one or more light source 3841 configured to shine or project light into the user's eye being tested. In some embodiments, light source 3841 may be communicatively linked to a processing unit so as to be controlled (i.e. be turned on/off or blink) either by the patient and/or operator, for example via a control interface (not shown), or according to a pre-programmed pattern. In some embodiments, a pre-programmed pattern may be synchronized with images or optotypes shown in a testing sequence. In some embodiments, light source 3841 may be a LED light source or similar. In some embodiments, one or more light sources 3841 may be movable (i.e. translated and/or rotated), for example via one or more actuators or similar.

The exemplary flow diagram of FIG. 39 illustrates how a variety of tests or assessments may be administered, according to one embodiment. These may include, as an example only and without limitation: eye movement tracking addressing an abnormality in the saccades 3905, gross motor monocular/binocular eye movement issues assessment 3910, near point of accommodation (NPA) assessment 3915, near point of convergence NPC 3920, pupillary assessment 3925.

Thus, in different embodiments, the detection of a cognitive impairment in a patient may be based on running or executing a series of tests or assessments (for example assessments 3905 to 3925) on refractor 3001 and associating with each individual assessment a quantitative value or “score” based on the degree of departure from a known baseline which would correspond to the values expected from a “normal” or cognitively unimpaired individual. The skilled technician will understand that different ways to quantify such a departure may be used, without limiting the scope of method 3900. Moreover, the baseline for each assessment or test may be derived either individually (e.g. measured from the same patient prior to a possible cognitive impairment event), derived from a group of individuals (e.g. by averaging measurements made on two or more healthy patients prior to any of them having experienced a possible cognitive impairment event), or from a more general pre-defined value based on the known medical literature. In some embodiments, a pre-recorded baseline assessment may be stored on remote server or database 3059 and retrieved at a later time when another series of assessments are made.

In some embodiments, gross motor monocular/binocular eye movement issues may be evaluated by using light field display 3003 to display moving an image or target and recording the user's response in real-time via one or more camera 3841.

In some embodiments, the near point of accommodation (NPA) may be evaluated (NPA assessment 3915). In some embodiments, a traditional “push” test may be performed monocularly, wherein optotypes or images are generated with the appropriate size for near vision testing (for example 0.4 M or 0.5 M) and are then virtually or perceptively moved towards the eyes using corresponding light field display pixel adjustments until they are perceived as blurry by the user. In some embodiments, a binocular embodiment of refractor 3001 (for example the embodiment of FIG. 35 or similar) may be used to evaluate both eyes one after the other. In some embodiments, such a test may be performed with phoropter 3001 using the best-known vision correction parameters for the patient and adding an additional dioptric power (e.g. +3D or else), for example via refractive components 3807, such as an electrically tunable lens or similar.

In some embodiments, near point of convergence NPC may be evaluated (NPC assessment 3920). This test may be used to measure the distance from the eyes for which both eyes may focus without double vision occurring. Thus, using refractor 3001 an image may be perceptively moved towards the user via light field display 3003 and/or refractive component 3007. In some embodiments, a binocular embodiment of refractor 3001 may be used to test both eyes simultaneously. In some embodiments, a pupillary assessment may be done (e.g. pupillary assessment 3925). In some embodiments, the pupil may be evaluated while the user is looking at light source 3841. In other embodiments, an image composed of several pixels of display 3003 may be lit up and moved around instead. The pupillary assessment may include pupil assessment data such as the size, shape of the pupil and/or static and dynamic aspects of the pupillary light reflex. In some embodiments, pupillary assessment data acquisitions may be done sequentially in both eyes.

In some embodiments, at step 3975, one or more of these quantitative scores may be combined into a single global cognitive impairment detection or testing score 3995 which may be indicative or suggestive that the patient is cognitively impaired. In some embodiments, score 3995 may be in the form of a binary score (e.g. yes or no), based for example on one or more thresholds defined at step 3975 for each test. In some embodiments, score 3995 may instead confer a degree of certainty quantifier such as a probability or similar. Finally, the score may be communicated to the patient or operator and/or recorded to be referred to at a later date.

While some tests may comprise a comparison with baseline values, various embodiments relate to the performance of tests without a baseline. While further description of selected tests will be discussed below, various embodiments relate to systems operable to perform tests related to oculomotor dysfunction/visual axis alignment, saccades and predictive saccades, smooth pursuit, range of motion, visual oculomotor reflex (VOR), pupillary response, comfort/discomfort level, sensitivity, or tolerance threshold, amplitude of accommodation, convergence insufficiency/excess, optokinetic nystagmus, visual midline shift, auditory speech-in-noise assessments, and the like, with tests and/or test sequences that are customisable and/or upgradable over-the-air (i.e. wirelessly).

The following description will provide an overview of additional illustrative tests that may be performed using a portable cognitive impairment assessment device. In accordance with the flexibility and customisability of the embodiments herein disclosed, the systems and methods herein contemplated may make use of any number or combination of the following tests; however, it will be appreciated that following tests are not an exhaustive list of examinations that may be performed. Indeed, other assessments not herein explicitly described and known in the art as relevant to the evaluation of a possible cognitive impairment, and enabled by a light field-based eye-tracking and/or physiology sensing apparatus as herein disclosed, are also herein contemplated. Further it will be understood that such tests may be included, programmed, downloaded, or the like, as individual tests that may be performed using the systems and methods herein described, or as elements of more comprehensive or customised assessment routines that may be configured for particular applications. For example, a testing routine specific to the evaluation of F1 drivers may comprise a designated and/or customisable subset of the following, or other, available assessments. It will be understood that such a subset may be different from one that is typically applied with, for instance, a device typically used in an ambulance.

Non-limiting examples of tests that may be performed by the light field-based eye tracking assessment systems and methods herein described may include the following list of assessments. The skilled artisan will appreciate that various tests may be related to individual eyes or to both eyes of a user, and may or may not be self-paced, as preferred on a test-by-test basis or for a particular application. Further description of these exemplary tests will be provided below.
Test 1: Gaze-based Saccades Horizontal. Example: two white dots fixed in space on a black background that form a horizontal line if connected. (self-paced)
Test 2: Gaze-based Saccades Vertical. Example: two white dots fixed in space on a black background that form a vertical line if connected. (self-paced)
Test 3: Gaze-based Saccades Oblique. Example: two white dots fixed in space on a black background that form a diagonal line if connected.
Test 4: Random Saccades. Example: a white dot that appears at a random location on a 2D plane (or in a 3D volume) on a black background for designated duration (e.g. 1 s) which then disappears and reappears after a consistent or random duration (e.g. every 1 to 3 s).
Test 5: Predictive Smooth Pursuit. Example: the user follows a white dot as it moves along the contour of a visible circular shape/trajectory, the white dot lighting up a length of the contour as it moves. (point-guided, not self-paced)
Test 6: Non-Predictive Smooth Pursuit. Example: the user follows a white dot as it moves along an invisible pre-defined trajectory (non-predictive, as compared to Test 5 in which the trajectory is visible).
Test 7: Photophobia and Phonophobia. Example: presentation of light and sound at varying intensities.
Test 8: Alternating display of a narrow beam of light on each eye.
Test 9: Accommodation. Example: readable target approaching from far viewing until blurriness is reported.
Test 10: Near Point Convergence. Example: moving target approaching the patient from optical infinite until double vision is reported.

Further tests that may be performed using embodiments described herein may include, but are not limited to, Optokinetic Nystagmus Horizontal, Optokinetic Nystagmus Vertical, Spontaneous and Gaze-Evoked Nystagmus Vertical, Spontaneous and Gaze-Evoked Nystagmus Horizontal, Subjective Visual Vertical, Subjective Visual Horizontal, Positioning Nystagmus, Positional Nystagmus, and/or Caloric assessments.

In accordance with various embodiments, testing routines may be created or executed as means of rapidly assessing various metrics relevant to a particular application or activity. Table 1 provides a non-exhaustive list of various metrics that may be ascertained (optionally on an eye-by-eye basis), as well tests from the abovementioned list that may be useful in determining the metric or a value related to the metric. It will further be appreciated that, in accordance with various embodiments, the metrics and tests herein outlined may be achieved with or without spatial or temporal calibration. For example, raw data may be used in assessment in consideration of the time domain, for instance when using a 500 kHz resolution eye tracker, as described in, for instance, Samadani U. “A new tool for monitoring brain function: eye tracking goes beyond assessing attention to measuring central nervous system physiology”, Neural Regen Res. 2015; 10(8):1231-1233. Further, it will be understood that the various metrics, descriptions, units, and related tests are presented as examples, only. For instance, a unit of m/s may similarly be measures and/or reported as °/s, or similar. Similarly, a list of tests corresponding to a metric is not exhaustive, and it will be understood that a test may further measure other metrics than those for which it is listed in Table 1 below.

TABLE 1
Exemplary metrics of interest.

| Metric | Description | Units | Test |
|---|---|---|---|
| Fixation Duration | Length of fixation | ms | 1, 2, 3, 4 |
| Number of | How many fixations were recorded during | Integer | 1, 2, 3, 4 |
| Fixations | the test | value | |
| Saccadic Amplitude | Magnitude of the saccade recorded | ° or | 1, 2, 3, 4 |
| | | arcmin | |
| Saccadic Accuracy | Accuracy of eye movements relative to the | ° or | 1, 2, 3, 4 |
| | displacement of the stimuli | arcmin | |
| Saccadic Peak | Highest velocity reached during the task | °/s or | 1, 2, 3, 4 |
| Velocity | | arcmin/s | |
| Saccadic Latency | Difference in time between the appearance | ms | 4 |
| (or Catch-up | of the target to the beginning of the | | |
| Saccade) | saccade | | |
| Catch-up Saccade | Amplitude of error of position and velocity | % | 4 |
| Amplitude | of the eye with respect to target as sampled | | |
| | 120 ms prior to saccade occurrence | | |
| Saccadic Overshoot | Dispersion of the measured point when it | % | 1, 2, 3, 4 |
| or Undershoot | reaches the target point (e.g. too far/not far | | |
| | enough, and by how much) | | |
| Main Sequence | Relationship between saccadic duration | ms | 1, 2, 3, 4 |
| | and amplitude | [fn(°)] | |
| Initial Acceleration | Rate of change of eye velocity within 80 | m/s² | 6 |
| | ms of target moving from a static position | | |
| Pursuit Velocity at | Velocity at set time points relative to the | m/s | 5, 6 |
| Set Time Points | target or pursuit initiation | | |
| Pursuit Gain | Peak pursuit velocity relative to target | % | 5, 6 |
| | velocity | | |
| Pursuit Latency | Time between the target appearance to the | ms | 6 |
| | beginning of the pursuit | | |
| Pupil Diameter | Diameter of the pupil | mm | 1, 2, 3, 4, 5 |
| Pupil Gaze | Difference between the two eyes when | mm | 1, 2, 3, 4, 5 |
| Variation | plotting the gaze of a followed object | | |
| Eye Skewness | Right eye skewness a possible biomarker | ° | 2 |
| | for concussion/post-concussion. | | |
| Degree of Line | Straightness of tracked line when gaze | ° | 1, 2, 3, 4 |
| Straightness | alternated between two fixed points | | |
| Amplitude of | Range of optical power | diopters | |
| Accommodation | | | |
| Accommodative | Speed of reaction for eye to accommodate | cycles/ | |
| Facility | | min | |
| Accommodation | Stamina of accommodation to repetitive | slope | |
| Fatigue | changes | | |
| Light Sensitivity | Threshold of user to brightness | % | 7 |
| Sound Sensitivity | Comfort threshold of user to sound | % | 7 |
| | amplitude | | |
| Slow Component | Velocity of the eye during the slow phase | ° /s | |
| Velocity | of the eye motion | | |
| OKN Gain | Gain of SCV | — | |

In the context of assessing an individual for a potential TBI, testing, in accordance with some of the systems and methods herein described, may generally be divided into two steps: identifying eye movements by filtering gaze data of the user's response to different stimuli, and computing metrics (e.g. those described above in Table 1) based on the administered test to ascertain a quantitative set of values that may be compared with clinically validated benchmarks for clinician assessment.

To date, a large body of research has been dedicated to the identification of appropriate algorithms for assessing eye movements. For instance, Salvucci and Goldberg (Salvucci, D. D., Goldberg, J. H., “Identifying Fixations and Saccades in Eye-Tracking Protocols. Proceedings of the 2000 Symposium on Eye Tracking Research and Applications, 2000, pp. 71-78) have described five different algorithms in a taxonomy for comparative purposes. IV-T, I-HMM, I-DT, I-MST and I-AOI were compared and described based on spatial/temporal capabilities. The taxonomy concluded that I-HMM (velocity-based) and I-DT (dispersion-based) provided accurate and robust fixation identification which were locally adaptive (temporally sensitive). Hence, various embodiments herein described may relate to one or both of these algorithms, although embodiments will be understood to not be so limited.

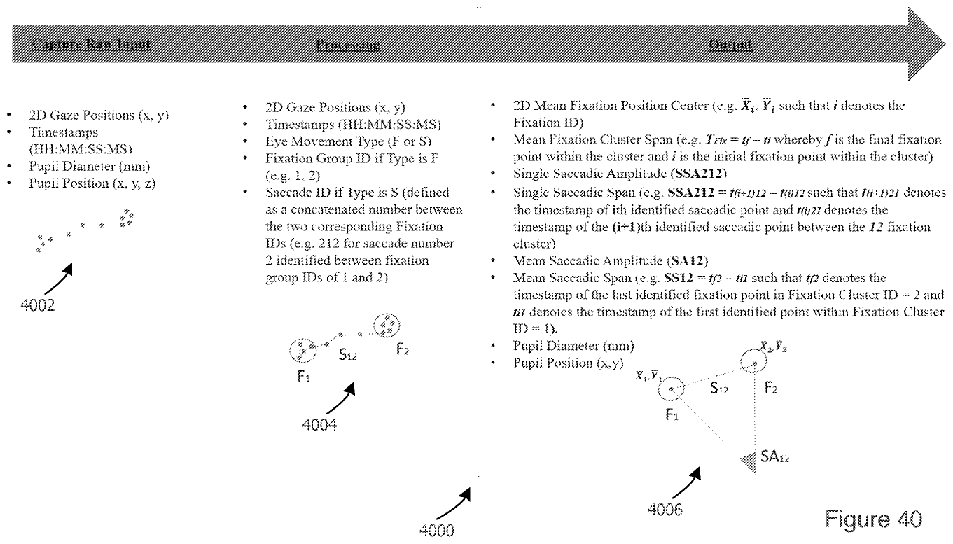

In accordance with various embodiments, gaze tracking data points acquired by an eye tracking system may first be identified as fixations and saccades, from which various metrics may be then be calculated. FIG. 40 shows an exemplary process flow 4000 summarising the identification of data points 4002 and exemplary associated output. Once a fixation cluster has been identified (e.g. F₁ and F₂ in FIG. 40), the individual points within that cluster, in some embodiments, may be augmented to or otherwise represented by a single mean value. Between two fixation clusters F₁ and F₂, a saccade S₁₂ (measured in, for example, degrees or arcminutes) may be identified, as schematically shown by processed data points 4004. In accordance with some embodiments, individual points may be recorded in accordance with an eye tracker's frame rate. As such, while a saccade may be considered as eye movement that is defined by two consecutive fixation points, it may also comprise several points. These points may, in comparison to fixation, be used to measure instantaneous velocities and acceleration variations. Hence, some embodiments relate to distinguishing between single and mean saccadic points. The sequential nature of data point acquisition and their spatial and temporal characteristics may enable such identification, and be used in the provision of output data 4006.

Various metrics, including, but not limited to, those listed in Table 1, may exhibit variations among patients suffering from, for instance, different grades of concussion or post-concussion syndrome. The thresholds and distinctions among these grades may be identified using both or either of established literature on the subject or clinical trials. In comparison with pre-conceived clinically validated data, a one-way univariate analysis of variance may be utilised. The following description accordingly provides various exemplary means of calculating the metrics presented in Table 1, which may, at least in part, be employed in such analysis, upon, for instance, execution of a process flow similar to that of FIG. 40.

Fixation Duration (FD): For or each of the identified fixation groups, the time between the first identified gaze point (1) and the last point (n^{th}) in, for example, milliseconds, may be calculated as:

$$F D = {T}_{n} - {T}_{1}$$

Number of Fixations (NF): Identification of the largest Fixation Group ID.

Saccadic Amplitude (SA): For each pair of the identified fixation clusters, the Euclidean distance (S) may be calculated as:

$$S = \sqrt{{( {\overline{X}}_{2} - {\overline{X}}_{1} )}^{2} + {( {\overline{Y}}_{2} - {\overline{Y}}_{1} )}^{2}}$$

where the saccadic distance measured in millimeters may be then be converted to degrees or arcminutes of the visual angle:

$$S {A}_{i j} = 2 * arctan ( \frac{S}{2 D} )$$

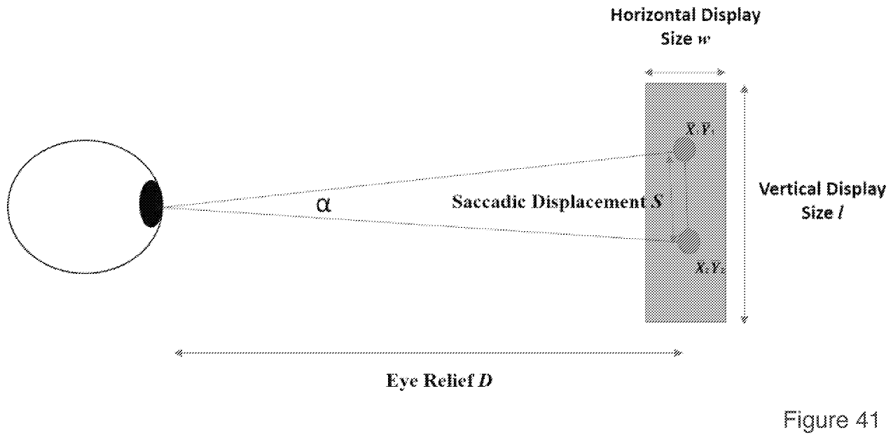

In some embodiments, distance measurements may be reported in pixels rather than in millimeters. In such cases, the saccadic amplitude in pixels may be calculated as:

$$Degree per Pixel = \frac{{tan}^{- 1} ( \frac{0.5 * l}{D} )}{0.5 * r}$$

$$Size in degrees = Size in Pixels * Degree per Pixel$$

where l is the vertical length of the display in millimeters, D is the eye relief, and r is the vertical resolution, as schematically depicted in FIG. 41. In accordance with one embodiment, an exemplary set of specifications for a cognitive assessment system may comprise a resolution r corresponding to a screen pixel resolution of 3840×2160 pixels, l=120.96 mm, and D=23 mm, yielding a conversion factor of 0.036031°/px (or 2.161 arcmin/px). The skilled artisan will appreciate that such calculations or analogues thereof may apply to either mean values, or to single saccadic recordings, for further analysis of saccadic peak velocities.

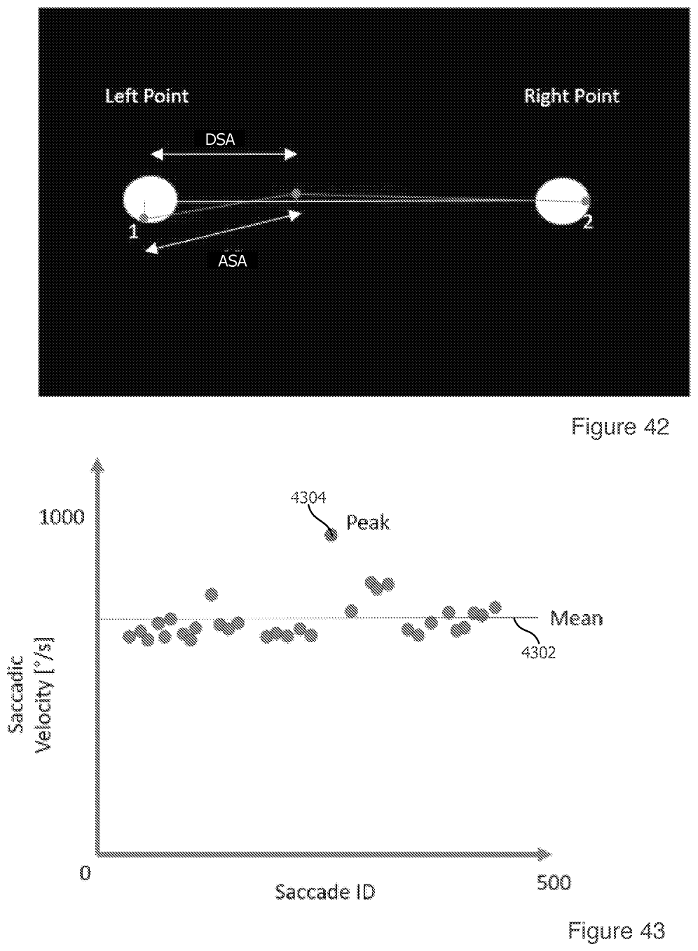

Saccadic Accuracy (SAC): For each mean saccade measured between two white dots displayed by the cognitive assessment system, SAC may be calculated as follows, where the actual and desired saccade amplitudes (ASA and DSA, respectively) are schematically illustrated in FIG. 42.

$$S A C = \frac{Actual Saccade Amplitude}{Desired Saccade Amplitude} = \frac{ASA}{DSA}$$

Single Saccadic Velocities (SV) and Saccadic Peak Velocity (SPV): Saccadic velocity may be calculated for each single saccade using corresponding timestamps and time spans T extracted therefrom as follows, whereby the largest SV may be considered as the SPV. FIG. 43 shows an exemplary plot of an illustrative distribution of single saccadic velocities and an associated mean velocity 4302, as well as the SPV 4304. In accordance with various embodiments, saccadic velocity data, distributions, and values extracted therefrom, such as those shows in FIG. 43, may have significance and be utilised in concussion diagnosis.

$${SV}_{i , i + 1} = \frac{{SA}_{i , i + 1}}{{T}_{i , i + 1}}$$

Saccadic Latency (SL): Saccadic latency may be measured, in accordance with some embodiments, in a test wherein a stimulus target is moved along a pre-defined, visible (i.e. predictable) trajectory at a constant (or, in other embodiments, a variable) speed. The different between timestamps corresponding to the initiation of the stimulus (e.g. via a timestamp noted from a dispatcher of the event stimulus) and the first recorded gaze point initiating the first saccade may yield the SL:

$$S L = {T}_{GAZE POINT} - {T}_{STIMULUS}$$

Catch-up Saccadic Velocity: Catch-up saccadic velocity may be measured for the first point identified as a saccade after a designated time (e.g. 120 ms) since the last identified fixation point.

Saccadic Overshoot/Undershoot (SOU): Upon completion of a test, the saccadic points may be identified. For example, after completion of Test 1, the x-coordinate of the saccadic points may be extracted and compared with the coordinates of the center of the target position to identify an overshoot/undershoot. In accordance with various embodiments, an overshoot may comprise the largest saccade magnitude past the target, an undershoot may comprise the last saccadic point before fixation outside of a target radius, and the Euclidean distance between the saccade points (x, y) and the coordinates of the closest white center (X_{w}, Y_{w}) may be calculated as:

$${d}_{S O U} = \sqrt{{( x - {X}_{w} )}^{2} + {( Y - {Y}_{w} )}^{2}}$$

wherein, if d_{SOU} is greater than the radius of the target circle R_{w}, then the overshoot or undershoot may be given as:

$$S O U = \vert \frac{{d}_{SOU} - {R}_{w}}{{R}_{w}} \vert * 100$$

In accordance with various embodiments, such a methodology may be employed in for either vertical or horizontal saccades (i.e. in consideration of the x or y coordinates), or both.

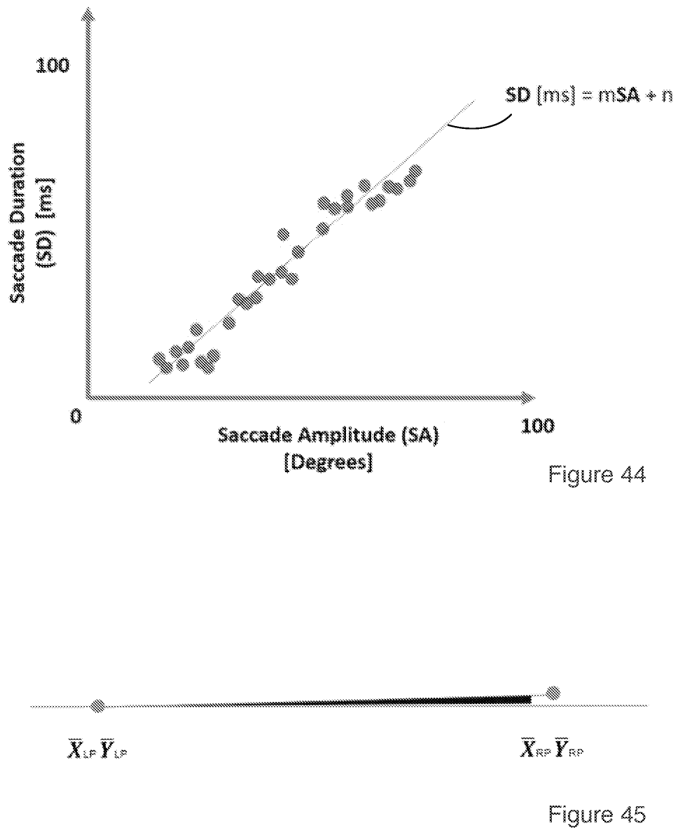

Main Sequence (MS): Main sequence (a term borrowed from astronomy) has been used in description of eye tracking due in part to the apparent fixed relationship that has been observed between the saccade duration and saccadic amplitude. For healthy individuals, the relationship is approximately linear. However, this has been reported to vary in concussed individuals. FIG. 44 shows an exemplary plot, in accordance with various embodiments, of such a correlation, where quantification of the linear correlation may be presented in terms of linear coefficients (m, n) for assessment of a potential impairment.

$$SA = mA + n$$

Further, a coefficient of determination may be presented, where ŷₗ is the predicted value, yᵢ is the actual value and y̅ is the mean.

$${R}^{2} = \frac{{Σ ( {\hat{y}}_{i} - {y}_{i} )}^{2}}{{Σ ( {y}_{i} - \overline{y} )}^{2}}$$

Upon moving a target in space, gaze points extracted from a participant as she follows a target moving at a constant speed, including initiation and maintenance, may be considered to be pursuit (i.e. no longer considered fixation and saccadic eye movements). However, similar methods to those for determining finding velocity and acceleration for saccades may be used, in accordance with various embodiments, to determine pursuit parameters, as described below.

Initial Acceleration (IA): Given a target that begins moving at a constant speed along a predefined route, the eye, upon the first motion of the target, typically follows the target after a certain latency (this may be considered as a parameter related to smooth pursuit). This may be measured, in accordance with various embodiments, a given duration (e.g. 20 ms) after the first identified saccade following the most recent fixation point. In one embodiment, the span of the saccades after the last identified fixation points may be calculated, with consideration only of, for instance, the saccade points noted after 20 ms. Conversely, the initial acceleration may be measured within a designated timespan (e.g. 80 ms from saccadic initiation, or from the 20 ms duration following motion), with only saccades within that span being considered. As such data is represented as single saccadic points, the velocities may be computed from the SV values described above. In one embodiment, the initial and final saccadic velocities within, for instance, the 80 ms span, are used to calculate IA as:

$$I A = \frac{S {V}_{f} - S {V}_{i}}{Δ t}$$

Pursuit Velocity (PV): The velocity of a pair of gaze points recorded during a pursuit may be calculated similarly to that of SV. According to the duration of movement, and in accordance with various non-limiting embodiments, datasets may be divided as:
0-100 ms after target appearance: prediction or anticipation
100-200 ms after target appearance: ability of pursuit in the absence of visual feedback
200-1000 ms after target appearance: Peak Pursuit Velocity (PPV) expected
1000 ms—End of examination: normal velocity with respect to following targets.

Pursuit Gain (PG): In part based on the PPV, the pursuit gain may be calculated as:

$$P G = \frac{Peak Pursuit Velocity}{Target Velocity}$$

Pursuit Latency (PL): Calculated similarly to the saccadic latency above.

Pupil Gaze Variation (PGV): When gaze points per eye are available, the distance between the two points and the eye-pair gaze may be compared during examination.

Eye Skewness: Pupil positions may be used to measure the skewness of the eye. The variation of the interpupillary distance in single-line tests, such as vertical motion tests, may, in accordance with some embodiments, provide insight to eye skewness. From coordinates of the left (subscript _{L}) and right (subscript _{R}) pupils (x, y, z), the vector may be computed as:

$$( {x}_{L} - {x}_{R} , {y}_{L} - {y}_{R} , {z}_{L} - {z}_{R} )$$

while the angle formed between the vector and the transverse plane may be defined as:

$${tan}^{- 1} ( \frac{\sqrt{{y}^{2} + {z}^{2}}}{x} ) .$$

In some embodiments, an image may be constructed for one eye at the stereoscopic position considering the interpupillary distance of the patient. The other eye image may then be moved by the patient or the examiner until a sharp binocular image is reported. The mismatch of the image position for the eye may be recorded and used to find the optical axes deviations from the calculated skew-free eyes values.

Degree of Line Straightness: This metric may be calculated as the angle between the desired line and actual line formed between fixation points, as schematically illustrated in FIG. 45.

Amplitude of Accommodation (AOA): The range of optical power that the eye can be achieved via adjustment of focus, as measured in, for instance, diopters. This may comprise, in accordance with various embodiments of a light-field based cognitive assessment device system or method, a dioptric measure between the near point (N) and far point (F) of accommodation such that:

$$AOA = N - F$$

Accommodative Facility (AF): Also referred to as inertia of accommodation, AF may relate to the evaluation of how quickly accommodation can change for an individual. In accordance with various embodiments, quantification of this metric may relate to simultaneous use of a light field display and eye tracking system, wherein the speed of and alternation may be calculated as the difference in time between the last gaze point identified as a fixation at the near (or far) object or plane, and the last gaze point identified as fixation at the far (or near) object or plane. The number of cycles (corresponding to the motion from near-to-far object then back from far-to-near) may be recorded per minute. In accordance with some embodiments, only the final fixation points may be utilised, as the user may not saccade back to the initial object until a focus is achieved, which may be assumed to be established at the last fixation gaze point.

$$A F = {t}_{last fixation at Object 1} - {t}_{last fixation at object 2}$$

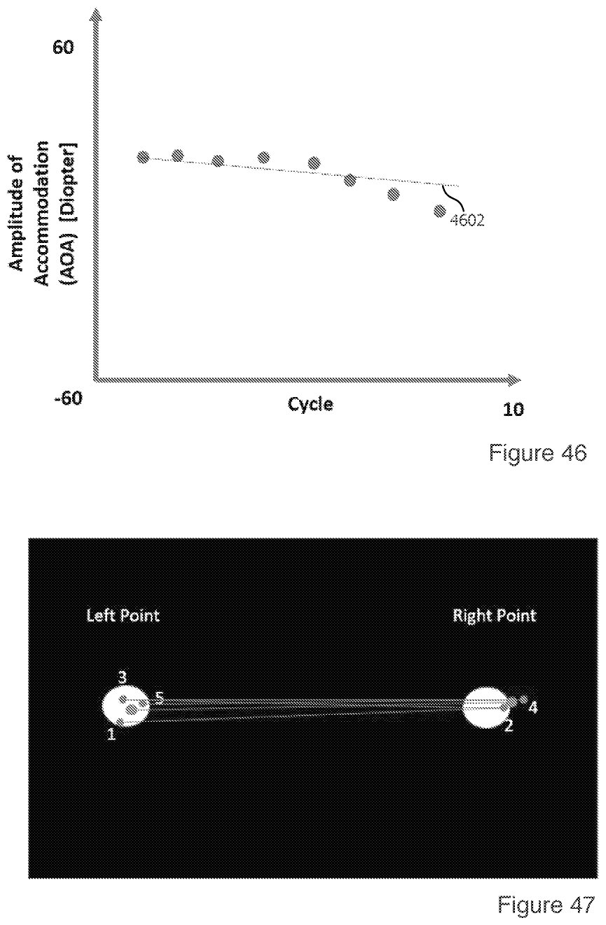

Accommodative Fatigue (AFat): AFat may be extracted through a repetition of the AOA test described above, whereby a decrease in the AOA recorded may signify fatigue of accommodation. FIG. 46 shows an exemplary plot of an illustrative graphical output that may be used is assessing AFat, wherein the slope of a linear fit 4602 (or other parameter associated with a fit curve) may provide an indication of a degree of fatigue (e.g. via a magnitude and sign of a slope).

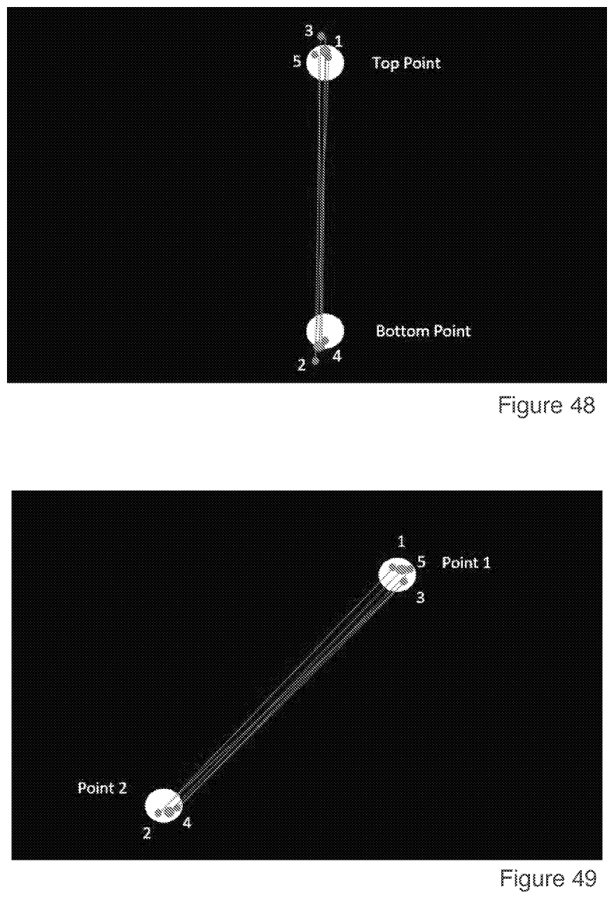

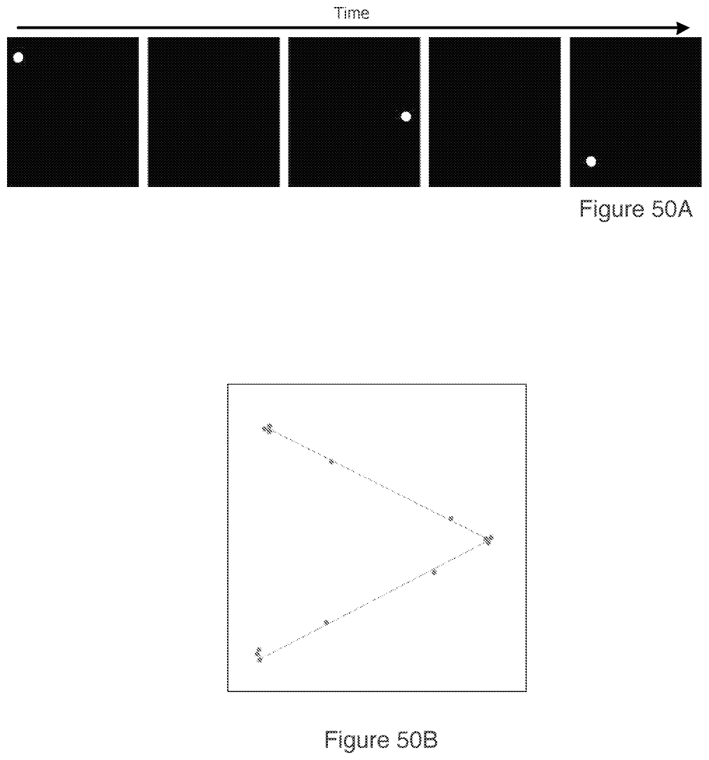

Having described various non-limiting examples of means and/or processes for calculating possible metrics of interest with respect to the assessment of a potential cognitive impairment, the following description will now elaborate on the exemplary tests 1 through 10 summarised above, with further discussion of test designs and with reference to exemplary metrics that may be at least in part determined thereby, in accordance with various embodiments. The skilled artisan will appreciate that these examples do not comprise an exhaustive list of assessments, and that the general scope and nature of the embodiments herein contemplated are not so limited. Further, it will be understood that, in accordance with some embodiments, metrics related to fixations and saccades as described below may be established using one or more appropriate tests, but not necessarily using others, even where it may be possible to do so. That is, specific tests may be designated as determining specific parameters without necessarily combining results between similar tests. For example, while it may be possible to extract saccade values from all tests within a testing profile, saccade assessment may comprise saccade characterisation using, for example, only tests 1 to 3 below, but not tests 4 to 6. Further, it will be appreciated that while the following tests comprise oculomotor assessments, various embodiments relate to performance of other tests related to other aspects of an individual's physiology (e.g. blood flow, oxygenation, or the like) that may be relevant to the diagnosis of a cognitive impairment.
Test 1: Two white dots fixed in space on a black background that form a horizontal line if connected. With reference to FIG. 47, the user may be asked to move their eyes between the two points (Left Point and Right Point in FIG. 47) as quickly and as accurately as possible. The data recorded may comprise a set of (x,y) coordinates, with their corresponding indices matched with a timestamp. The (x,y) coordinates, in accordance with various embodiments, may correspond to the 2D gaze points projected onto the display screens. The data may, in one embodiment, be filtered in real-time. In the example of FIG. 47, each white dot is 0.95° in diameter and 9.5° apart, and the test may last for a designated duration (e.g. 10 s). Metrics that may be determined form such a horizontal self-paced saccade test, in accordance with various embodiments, may include:
   Fixation Duration
   Number of Fixations
   Saccadic Amplitude
   Saccadic Accuracy
   Saccadic Peak Velocity
   Single Saccadic Velocities
   Main Sequence
   Pupil Diameter
   Pupil Variation
   Degree of Light Straightness
Test 2: Two white dots fixed in space on a black background that form a vertical line if connected. The user may be asked to move their eyes between the two points as quickly and as accurately as possible, as schematically shown in the illustrative example of FIG. 48. The data recorded may comprise a set of (x,y) coordinates and their corresponding indices matched with a timestamp. The (x, y) coordinates may correspond to the 2D gaze points projected onto the display screens. The data may, in some embodiments, be filtered in real-time. In the example of FIG. 48, each white dot is 0.95° in diameter and separated by an angle of 9.5°, while the test may last for, for instance, 10 s. Saccades identified within the context of this test may be identified as vertical self-paced saccades. Non-limiting examples of metrics that may be extracted from such a test may include:
   Fixation Duration
   Number of Fixations
   Saccadic Amplitude
   Saccadic Accuracy
   Saccadic Peak Velocity
   Single Saccadic Velocities
   Main Sequence
   Pupil Diameter
   Pupil Variation
   Degree of Light Straightness
   Eye Skewness
Test 3: Two white dots fixed in space on a black background that form a diagonal line if connected. The user may be asked to move their eyes between two points as quickly and as accurately as possible, as schematically illustrated in FIG. 49. The data recorded may comprise a set of (x, y) coordinates, with their corresponding indices matched by a timestamp. The (x,y) coordinates may correspond to the 2D gaze points (e.g. points 1 to 5 in FIG. 49) projected onto the display screens. In accordance with some embodiments, data may be filtered in real-time. In the embodiment of FIG. 49, each white dot is 0.95° in diameter and separated by an angle of 13.26°, wherein the value of the distance between the two dots may be chosen to correspond to analogous x and y separations between points in Tests 1 and 2 described above. Accordingly, such a diagonal self-paced saccade assessment, in accordance with various embodiments, may provide additional complementary data to (or replace) Tests 1 and 2 above in conventional saccade assessments. Metrics established from this test, in accordance with various embodiments, may include:
   Fixation Duration
   Number of Fixations
   Saccadic Amplitude
   Saccadic Accuracy
   Saccadic Peak Velocity
   Single Saccadic Velocities
   Main Sequence
   Pupil Diameter
   Pupil Variation
   Degree of Light Straightness
Test 4: A white dot randomly appears in a black background, then disappears. Schematically illustrated in FIGS. 50A and 50B, such a test may relate to the user moving their eyes as quickly as possible to a target stimulus (white dot) as it appears on the screen, and may relate to saccades-to-command metrics. Data may comprise a set of (x,y) coordinates, with their corresponding indices matched with a timestamp. The (x,y) coordinates may correspond to the 2D gaze points projected onto the display screens. In accordance with one embodiment, data may be filtered in real-time. In the example of FIG. 50A, each white dot is 0.95° in diameter and appears for 1 second, with time increasing from left to right. Both the spatial and temporal characteristics of the appearing/disappearing while points (i.e. their positions and durations of persistence) may, in accordance with various embodiments, be generated randomly. In some embodiments, a range of times may bracket otherwise randomly selected durations (e.g. points may persist for randomly selected durations between 1 and 3 seconds). Such randomness in duration and position may, in accordance with some embodiments, improve repeatability and/or reproducibility of such assessments while minimising effects of user prediction. In accordance with various embodiments, such an assessment may last between 20 s and 40 s. Alternatively, or additionally, assessment duration may relate to the appearance of a designated number of appearances of points with a random position and duration (i.e. ensuring that 10 points will appear, optionally for a total assessment duration of between 20 s and 40 s). FIG. 50B shows exemplary gaze tracking data points acquired as the user tracked the randomly selected white dot positions of FIG. 50A. Exemplary metrics that may be evaluated may include:
   Fixation Duration
   Number of Fixations
   Saccadic Amplitude
   Saccadic Accuracy
   Saccadic Peak Velocity
   Saccadic Latency
   Single Saccadic Velocities
   Saccadic Overshoot or undershoot
   Catch-up Saccade
   Main Sequence
   Pupil Diameter
   Pupil Variation
   Degree of Light Straightness

It will be appreciated that variants of such saccade tests may be performed using a portable cognitive impairment assessment device, in accordance with various embodiments. For instance, an anti-saccade test may be performed, wherein the subject is asked to look at the direction opposite to that of the appearance of a stimulus (e.g. a white dot). In one exemplary test, the subject is may be asked to imagine a mirrored point directly opposite to the dot that appears and fixate on it. Parameters such as those employed by O'Driscoll (O'Driscoll G A, Lenzenweger M F, Holzman P S. “Antisaccades and Smooth Pursuit Eye Tracking and Schizotypy, Arch Gen Psychiatry. 1998; 55(9):837-843) may be used, including the provision of anti-saccades measurement via display of a dot on the screen center for a period of 800 to 1200 ms which then disappears to reappear at 12° to the left/right side for 100 ms, wherein the subject then looks at the estimated mirrored point.

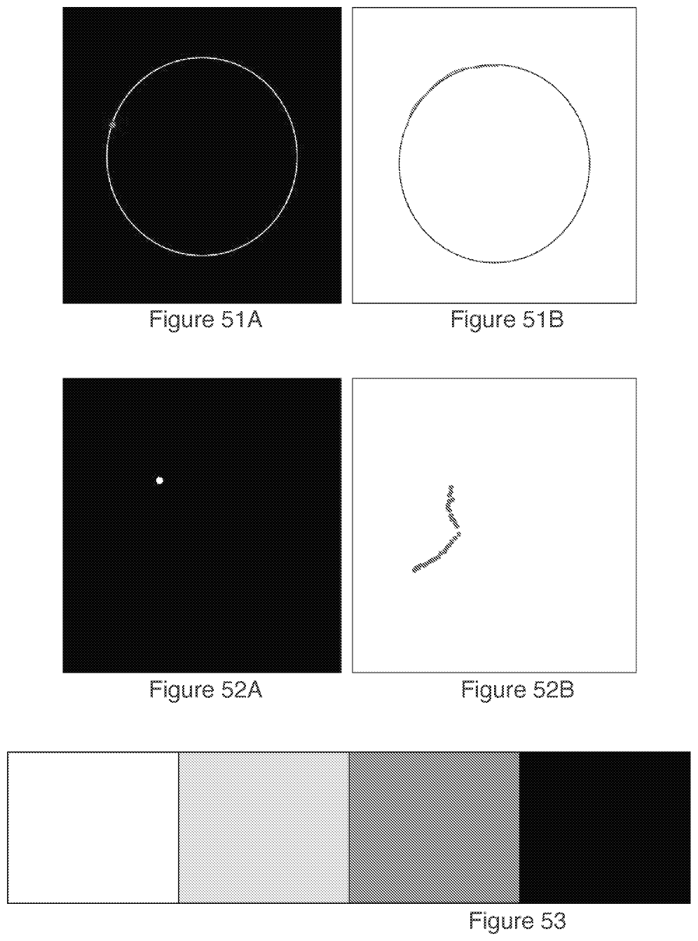

Test 5: Eyes follow the contours of a circular shape. This test, schematically illustrated in FIGS. 51A and 51B, may be point-guided (i.e. not self-paced) such that a point moves along the edge(s) of a shape(s), which, in accordance with one exemplary embodiment, lights up a point or segment of the edge as the point moves along the contour. This exemplary assessment may relate to predictive pursuits, and may relate the acquisition of metrics within a closed-loop state of smooth pursuit, with possible reporting/output relating to mean pursuit velocity, mean pursuit gain, maximum and minimum pursuit velocities, or the like. In such as assessment, the user may move their eyes to follow the dot as it moves along a circular path in FIG. 51A. The data output (FIG. 51B) may comprise a set of (x,y) coordinates, with corresponding indices matched by a timestamp. The (x,y) coordinates corresponding to the 2D gaze point projected onto the display screens may be filtered in real-time. In the embodiment of FIGS. 51A and 51B, the colored dot is 0.95° in diameter and moves at a speed of 25.13°/s, while the thickness of the circle was defined as 20% of the diameter of the colored dot (i.e. 0.19°). In one embodiment, the assessment may comprise two cycles of the dot traversing the contour of the circular trajectory (the circular trajectory having a diameter of 9.5° in FIGS. 51A and 51B). Exemplary metrics that may be established may include:
Pursuit Velocity at set time points
Pursuit Gain
Pupil Diameter
Pupil Variation
Test 6: A white dot moving along a pre-defined trajectory with no visual access to the trajectory itself. In this example, schematically illustrated in FIGS. 52A and 52B, pursuit gaze points (FIG. 52B) may be identified as non-predictive pursuits, wherein metrics evaluated and/or reported as output may comprise those related to the open-loop and closed-loop stages of smooth pursuit (e.g. pursuit mean velocity, mean pursuit gain, maximum/minimum pursuit velocities, mean/maximum/minimum pursuit latency, initial acceleration, etc.). In the example of FIG. 52A, the user may move their eyes to follow the white dot as it moves, when and where they observe it moving. In accordance with one embodiment the assessment may begin with no dot shown for a designated or random duration of time (e.g. 2 s), after which the dot may appear. The timestamp of the white circle appearance may define the beginning of the open-loop stage of smooth pursuit. The data recorded may comprise a set of (x,y) coordinates, with their corresponding indices matched with a timestamp. The (x,y) coordinates, corresponding to the 2D gaze points projected onto the display screens, and shown in the exemplary plot of FIG. 52B, may be filtered in real-time. In the exemplary embodiment of FIG. 52A, the dot is 0.95° in diameter and moves at a speed of 25.13°/s. The trajectory followed by the dot may, in accordance with various embodiments, require prior definition, or it may be generated randomly. Non-limiting examples of metrics that may be determined from such a test may include:
   Initial Acceleration
   Pursuit Velocity at set time points
   Pursuit Gain
   Pursuit Latency
   Pupil Diameter
   Pupil Variation
Test 7: Light and sound sensitivity. As an individual with a concussion may be sensitive to bright light and elevated volumes, various embodiments relate to performing a test via a light field-based cognitive impairment assessment device in which the individual is presented with variable levels of illumination and volume. For example, FIG. 53 schematically illustrates four possible light field display brightness levels. In one test, the user may be first presented with a dark screen (no illumination) which gradually increases in brightness until the individual reports discomfort (e.g. presented screens from right to left in FIG. 53). Conversely, a test may comprise presenting the user with a maximally or highly illuminated screen, which decreases in brightness until the user reports no discomfort (from left to right in FIG. 53). Variable brightness may be achieved via a pre-programmed ramp of display illumination (e.g. automatically), or via, for instance, a user-controlled knob for adjusting screen brightness until a level of comfort/discomfort is noted. While FIG. 53 shows four different brightness levels, it will be understood that any level of brightness within the possible illumination output from a light field display (e.g. up to the maximum output capability of the screen) may be employed in such a test. In accordance with various embodiments, a similar test may be performed with audio sound (e.g. via speakers or headphones), wherein the user reports when an audio level that comfortable/causes discomfort is achieved via automatic or user-controlled ramping of an output volume.
Test 8: Narrow beam of light in alternating eyes. In accordance with various embodiments, the assessment device may record, analyse, and/or be used by a physician to monitor how an individual's pupil diameter, pupil behaviour, pupil variation, or the like, behaves in response to intermittent or alternating illumination as governed by activation of pixels of a digital display and transmission of light through light field shaping elements.
Test 9: Readable target approaching until blurriness is reported. As schematically illustrated in FIGS. 54A and 54B, a light field may be generated that provides for an object 5402 comprising symbols (e.g. a Snellen chart, letters, numbers, or other like identifiable or recognisable characters) at various depth planes. Unlike conventional 2D display screens which isotropically emit light (e.g. with limited to no control over perceived object depth), a light field display may be operable to control or manipulate light such that light reaching the retina provides an image of the object as if rays had originated from an object at a designated depth. Accordingly, an object 5402 may be presented from, for instance, sequentially approaching (or retreating) depth planes within the field of view while displaying characters until characters or the object 5402 itself appears blurred, as schematically illustrated in FIGS. 54A and 54B. While grid lines in FIGS. 54A and 54B are shown for illustrative purposes to provide a sense of changing depth, various embodiments may alternatively relate to the display of an object 5402 without such depth cues, as the light field display inherently enables the display of objects on designated planes without requiring such cues. Further, it will be appreciated the object 5402 may itself constitute the characters to be read (rather than comprising a surface on which characters are displayed). In accordance with various embodiments, a user may report when blurriness is observed for recording and analysis by the cognitive assessment device or specialist. Such assessments may be performed in accordance with conventional parameters for assessment. For instance, the object 5402 may be displayed as a rectangular or “finger-like” shape (e.g. 90 mm length, 15 mm in width) which appears to approach the user from 1 m distance to 0.05 m distance as a speed of approximately 10 mm/s. In some embodiments, features to be read (e.g. letters) may have a width and height of approximately 10 mm.

Such tests may be further employed to determine accommodative fatigue. For example, the abovementioned target 5402 may be presented and fixated upon at a fixed distance until the target appears clearly while dioptric powers are increased/decreased via, for instance, exchange of lenses in the field of view.

Variants of such a test may be further employed to assess relative accommodation. For example, upon applying a perception adjustment to correct for a user's current visual acuity, a target may be presented at a designated distance (40 cm) from the eyes. In one embodiment, a lens may be presented in −0.25 D increments until the target is perceived as blurry. The total value of the lenses added to reach that point may comprise the PRA value. High PRA values (greater than or equal to 3.50 D) may, in some embodiments, provide a biomarker of a disorder related to accommodative excess, while those with accommodative insufficiency may exhibit PRA values below −1.50 D.

In some embodiments, relative accommodation tests may be performed once vision is corrected (e.g. via the light field display) while a small target is presented at a distance equivalent to, for instance, 40 cm from the eyes. Corrections of +0.25 D increments may be presented (e.g. via lenses or re-rendering of visual content) until the target is perceived as blurry. The total value of the lenses added to reach this point is the PRA value. High NRA values (above +2.50) might be evidence of uncorrected hyperopia or latent hyperopia.

Accommodative facility may further be assessed through such a test, or a similar test. Such assessment may be performed both monocularly and binocularly. In one embodiment, the subject may look to a small presented target through plus or minus lenses, or simulation thereof via light field rendering techniques. Once the target becomes clear, dioptric shifts may be applied (e.g. via light field rendering). This operation may be repeated several times, with assessment comprising a metric such as cycles per minute. Such assessments may typically comprise measurement with a ±2 D lens monocularly. Typical values for an average monocular accommodative facility, approximately 11±6 cycles per minute, may be used for comparison.

Assessment may comprise, for instance, comparison with previous examinations of the individual, with control groups of known healthy and concussed individuals, or the like. In some embodiments, metrics or other data may be reported as a comparison to an age-adjusted normal amplitude of accommodation calculated with, for instance, Hofstetter's formula (i.e. minimum monocular accommodative amplitude=15D−0.25×age).

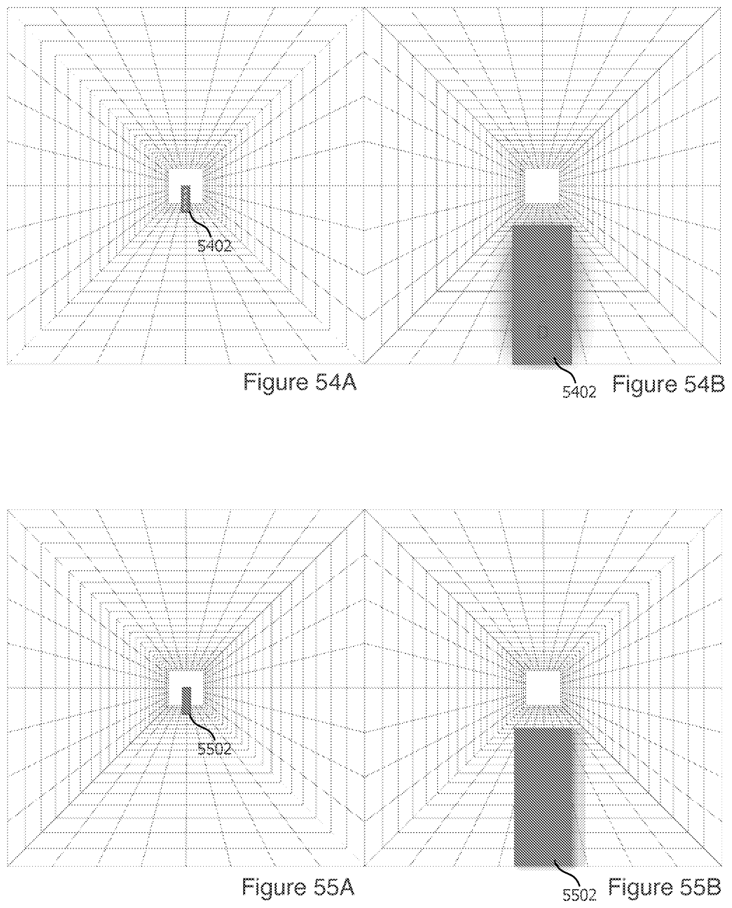

Such assessments may contribute to establishing metrics related to, for instance:
Amplitude of Accommodation
Accommodative Fatigue
Pupil Diameter
Pupil Variation
Accommodative Facility
Test 10: Moving target approaching the patient until double vision is reported. Schematically illustrated in FIGS. 55A and 55B, an object 5502 may be provided by a light field display at, for instance, optical infinity, and then moved forward (i.e. towards the user). A user may, in accordance with some embodiments, follow the target as it moves from far-to-near, and report on when double vision is observed. Reporting by a cognitive impairment system may comprise, for instance, a dioptric power that was displayed when double vision was observed. Such assessments (e.g. near point convergence tests) may facilitate establishment of metrics related to, for instance:
   Amplitude of Accommodation
   Accommodative Fatigue
   Pupil Diameter
   Pupil Variation
   Accommodative Facility

Non-limiting examples of further tests that may be performed with a portable cognitive impairment assessment system will now be described.

An optokinetic nystagmus horizontal test may be performed which mimics the rotating motion of a drum typically utilised within the OKN Full-Field testing setup. The setup is conventionally centered above the subject's head in a dark cylindrical room consisting of a light projecting bars onto the cylindrical walls, which may be simulated using embodiments herein described. The bars may simultaneously be sustained at a luminance of, for instance, 10.1 and 5.1 cm/m2 for the light and dark portions, respectively (i.e. at a 2:1 contrast ratio). The bars may initially be displayed at 0 degrees per second, which may be increased at a constant acceleration (e.g. 2 degrees per second squared) to reach a maximum velocity of, for instance, 55 degrees per second, to be maintained for a designated duration (e.g. 2 seconds) before decreasing back to 0 degrees per second. Such a test may be performed with eye sampling performed at, for instance, a minimum of 120 Hz. To better mimic a conventional test, and in accordance with one embodiment, a cylindrical drum (e.g. 25 cm in diameter and 45 cm in height with equally spaced black and white stripes) may be simulated above the subject's head in a virtual reality scene, with rendered lighting projecting onto cylindrical walls of the environment (e.g. 2 m diameter). A Spatial Frequency (SF) may be used to define the distance between the black and white bars. A number of SF values may be used and compared to each other during/after assessment (e.g. 0.022, 0.047, 0.094 and 1.5 cycles per degree). In general, higher spatial frequencies may show lower SCV values. Participants with a concussion or cognitive impairment may show lower values of SCV and SF. Accordingly, and in accordance with some embodiments, four tests may be performed to achieve a desired output. Upon conducting the clinical trials, a threshold may be implemented, wherein a single test may be preferentially performed.

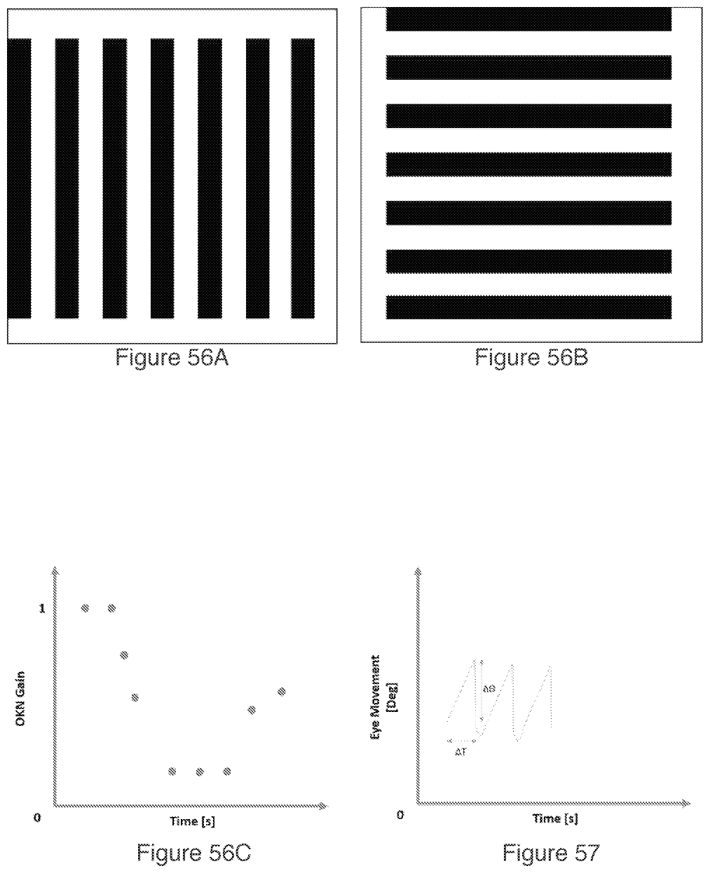

A schematic of an exemplary rendering that may be used for such an assessment is shown in FIG. 56A. In accordance with one embodiment, the slow component velocity may be plotted against time among a variation in the SF. Further, OKN gain metrics may be assessed by such a test. Similarly, and as schematically illustrated in FIG. 56B, optokinetic nystagmus vertical tests may be performed using similar procedures while employing a vertical formation. An exemplary plot of OKN gain versus time is illustratively shown in in FIG. 56C, where gain may be calculated as the SCV divided by the “drum velocity”.

A spontaneous and gaze-evoked nystagmus vertical test may by performed in which the subject is asked to look straight ahead while no image is initially displayed. Live video feedback of the eyes, as well as a velocity of pupil position, may be measured. The practitioner may then include a marked point with designated dimensions placed vertically upwards or downwards (e.g. 30° upwards or downwards). Such placement may, in some embodiments, be decided by the practitioner using a graphical interface or digital application associated with the assessment system. The subject may then be asked to look at the stimulus based on practitioner input. The difference between the velocity components with and without gazing may provide insight as to the source of a potential issue (e.g. peripheral or central). FIG. 57 shows an exemplary plot of eye movement versus time that may be employed in such an assessment, which may be used to establish metrics related to, for instance, slow component duration, slow component velocity, and vertical pupil position with respect to time. It will be appreciated that similar assessments may be enabled via embodiments herein disclosed for spontaneous and gaze-evoked nystagmus horizontal, wherein a stimulus is provided by the device at a designated angle rightward or leftward.

A subjective visual vertical test may be performed to assess a subject's ability to properly perceive vertical lines. In such an assessment, a light bar of designated dimensions (e.g. 30 cm by 1 cm) may be rendered at the screen and to be perceived as originating from a designated distance away (e.g. 1.5 m away). Such a test, in accordance with one embodiment, may comprise initially providing the bar at a designated angle. The practitioner may then rotate the bar via a user interface (e.g. via a digital application associated with the device), while the subject is asked to inform the practitioner via audio to identify when the light bar is vertically straight. The angle between the line and the vertical axis may then be reported. While such assessments are conventionally performed using a form of bucket, various embodiments relate to the use of such a test in a VR scene. It will further be appreciated that a Subjective Visual Horizontal assessment may be similarly performed using embodiments of a portable cognitive impairment assessment system as herein described.

A caloric test is typically employed to manipulate the vestibulo-ocular reflex eye movement through stimulation of the ear-canal with a small amount of material (e.g. a fluid, such as water) with hot and cold temperatures. The temperature differential between the human body and the injected material results with a nystagmus response of the eyes via the afferent nerves of the semicircular canal (three fluid-filled structures in the middle ear that act as sensors for spatial orientation). When a relatively cold temperature (e.g. 30°) is induced, a fast-beating nystagmus occurs in a direction opposite to the ear which was utilised, while the slow-beating nystagmus occurs to the contralateral side. With relatively warm water (e.g. 40°), the opposite typically occurs. Nystagmus beats occur over a timespan of 30 s to 45 s, whereby the amplitude may increase within that time span. The amount of water delivered may be approximately 250 cc over a 25 s to 30 s period with the subject's head placed at a designated angle (e.g. 30°). A head-mounted cognitive impairment assessment system, in accordance with various embodiments, may be outfitted with accessories to perform such caloric assessments. For example, material (e.g. relatively warm/cool water) may be introduced to the subject's ears via the head-mounted device, with tests performed with a designated wait time (e.g. 5 min) between material introduction in the ears. Such assessments may be performed by acquiring a baseline for the subject. Acquired metrics from such an assessment performed using a head-mounted cognitive assessment device may relate to, for instance, amplitude of the horizontal movement, time span of the fast component of a nystagmus, time span of the slow component of a nystagmus, slow-component velocity, and/or fast-component velocity.

Relative accommodation assessments may also be performed using various embodiments of a head-mounted cognitive impairment assessment system having light field functionality. For instance, positive relative accommodation (PRA) may be assessed as the subject's ability to accommodate a while maintaining a target clearly with binocular vision. Similarly, negative relative accommodation may be assessed.

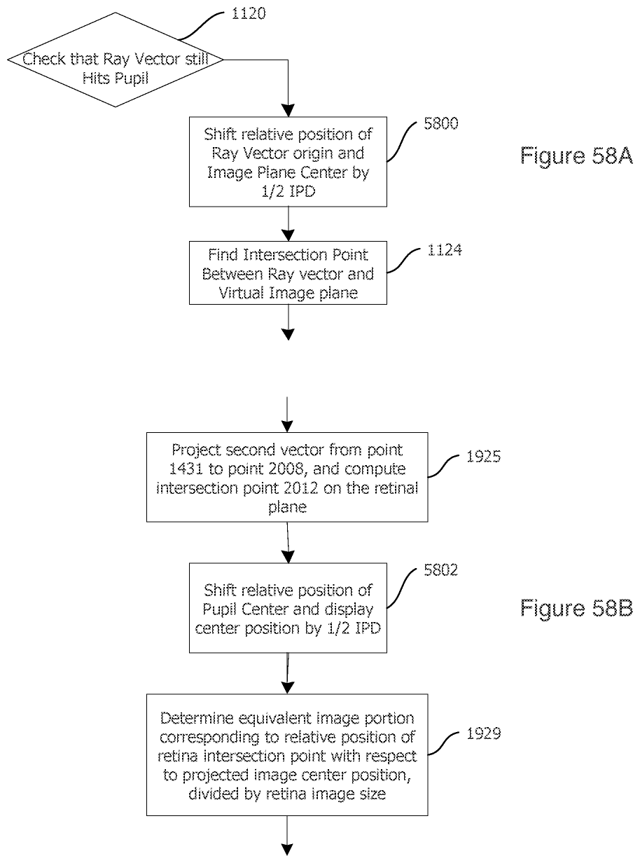

In some embodiments, for example in the binocular embodiment of FIG. 58A or 58B, the light field rendering methods described above may be slightly modified to account both eyes viewing the same image. Therein, the same image is used by both light field displays but the light field generated therefrom is shifted accordingly for each eye so as to appear centered therebetween.

This may be used, for example, for any kind of vergence-related cognitive impairment tests, including for example NPC assessment 3920 or Test 10 described above.

In some embodiments, the general position or location of the (light field image) may be re-centered between the eyes (i.e. shifted horizontally by a value equal to half the interpupillary distance).

In one embodiment and as illustrated in FIG. 58A, methods 1100 and 2400 may be modified so that before extending ray 1417 to intersect with the virtual image plane 1405 in step 1124 to identify the image portion, the origin point of ray 1417 (e.g. point 1431) may be shifted horizontally by half the interpupillary distance (IPD) (to the right if right eye, or to the left if left eye) in a preceding step 5800. Then ray 1417 is projected from this new location (but with the same orientation) to intersect with virtual image plane 1405 as discussed above. Inversely, the same result would be achieved by horizontally shifting the location of virtual image plane 1405 instead by the same distance, but in the opposite direction.

Similarly, methods 1900 or 2700 may be modified to also shift the light filed image so that it is perceived by each eye as originating somewhere in between. In this case, a new step 5802 is added in between steps 1925 and 1929, wherein the center position of the image on the retina (point 2020) is shifted horizontally so as to model the image center location 2018 being equally shifted by half the IPD.

In some embodiments, the IPD may be measured in real-time (via one or more cameras 3017 or a displacement sensor) or a pre-determined value may be used. The pre-determined value may be an average value, for example a value corresponding to the patient's demographics, or it may be the patient's IPD that has been measured prior to using the device.

In some embodiments, a more general implementation may be considered. For example, in accordance with one exemplary embodiment, a general light field image offsetting approach will be described below that can be used to offset the image for left and right eyes in monocular and stereoscopic vision settings by assigning a translation vector to the original image.

### Shifted/Stereoscopic Image Projection to a Retinal Image

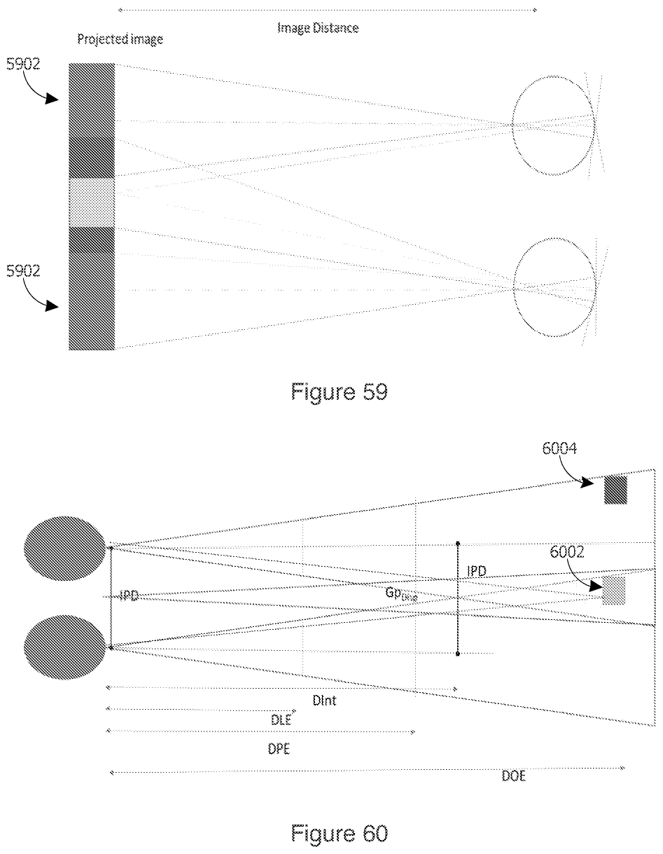

As illustrated schematically in FIG. 59, the original projected image/object plane (blue rectangles 5902) is assumed to be parallel to the pupil and retina planes. For a displaced image/object, where the image/object is not changing in size, a single vector for each eye is sufficient to describe the translation. Also, since the image/object distance to the eye (DOE) is an input parameter in Z direction, the translational vectors are in the x,y coordinates. The vectors for left and right eyes (L,R superscripts) can be defined as:

$${T}_{I}^{L , R} = {t}_{x}^{L , R} \hat{x} + {t}_{y}^{L , R} \hat{y} .$$

In some embodiments, the axis of rotation of the eye may be assumed to be located at the center of the eyeball or Eyeball Center (EBC). The retinal image plane may be defined to be parallel to the pupil at an eye depth offset distance and the optical axis may be defined to be equal to the central pupil normal vector, which passes from the center of the pupil center to the eyeball center. Then the optical axis vector (O̅A̅^{L,R}) may be found using:

$${\overline{O A}}^{L , R} = {T}_{I}^{L , R} + ( D O E + \frac{E D}{2} ) \hat{z} .$$

In addition, the Retina Image Origin location [symbol] RIO [symbol] ^{L,R} may be defined as the intersection point of the optical axis with the retina plane, while the Image Origin [symbol] IO [symbol] ^{L,R} may be defined as the intersection point of the optical axis with the image plane. Since the rotation of the eye happens around the EBC which is fixed in space, it can be taken as a reference point. The coordinates for this point can be easily found relative to the pupil coordinates in the system. Then the retinal image origin (RIO) is given by:

$${〈 R I O 〉}^{L , R} = {〈 E B C 〉}^{L , R} - \frac{{\overline{O A}}^{L , R}}{\vert \overline{{\overline{O A}}^{L , R}} \vert} \frac{E D}{2} .$$

Since the image is projected with rays nodal to the pupil center, the projection between parallel planes is done by scaling the coordinates around the nodal point. Offset vectors (OV) may be defined that map the image coordinates around the optical axis and scales it to the unshifted retinal plane coordinates (USRP), for example via:

$${〈 O V 〉}_{USRP}^{L , R} = - \frac{E D}{D O E} ( {〈 I C 〉}^{L , R} - {〈 I O 〉}^{L , R} ) .$$

To get the actual offset vector on the on the retinal plane gazing at the shifted image, this vector has to be projected onto the actual shifted retinal plane (no subscript) are projected, as shown below:

$${〈 O V 〉}^{L , R} = {〈 O V 〉}_{USRP}^{L , R} - \frac{{〈 O V 〉}_{USRP}^{L , R} \cdot \overline{O {A}^{L , R}}}{{\vert \overline{O {A}^{L , R}} \vert}^{2}} \overline{O {A}^{L , R}} .$$

Then the retinal image coordinates may be calculated using:

$${〈 R I C 〉}^{L , R} = {〈 O V 〉}^{L , R} + {〈 R I O 〉}^{L , R} .$$

For a stereoscopic view, the translational vectors may be calculated for the image coordinates in such a way that the left and right eyes images coincide. For moving the image within certain angular distance relative to a point like the eye pupil, the image translation vector may be calculated using tangent relationships in the corresponding directions and added together. Following this, the rotation of the light field display to the new optical axis may be computed as will be described below.

In rotated coordinates, the pupils may be set on the optical axis where the x,y coordinates of the pupil coincide the retinal image center. The z coordinate is offset from the retinal image center by −ED in the z direction.

### Shifted/Stereoscopic Image Projection Based on a Unified Implementation

In some embodiments, a more general treatment of the light field rendering may be used. This may include, for example, a more generalized version of method 1100 described above, in which planes are not parallel to each other. It may also include other light field rendering methods, for example the unified implementation described in PCT application PCT/IB2021/051868, the content of which is hereby incorporated by reference. In the abovementioned reference, a phase element or virtual optical element is considered during the ray-tracing so as to model any number of optical aberrations. The refracted or deviated rays generated from those phase elements may then be propagated backwards (towards the pixel display) to intersect with a virtual object at a designated location.

### Accommodation

For stereoscopic vision, the eye power generally changes so to allow the eye to focus the image on the retina in synchronization with the triangulation of the stereoscopic image.

As shown schematically in FIG. 60 is an exemplary elaboration where the virtual object 6002 is perceived by both eyes while the virtual object 6004 falls in the monocular region. For wide angle emission display where view for one eye can reach the other eye, a physical barrier can be used in between. Assuming that the gap between the two binoculars is given by G_{Disp} then the distance (D_{Int}) at which the displays projection intersect with zero gap is given by:

$${D}_{Int} = D P E \frac{I P D}{I P D - {G}_{Disp}} ;$$

where DPE is the pixels/display to eye distance and the binocular view width (W_{Bi}) is a function of DPO, the projected plane distance to pixels/display, =(IPD/DPE)DPO:

$${W}_{Bi} \approx I P D ( \frac{D O E}{{D}_{Int}} - 1 ) ; and \\ {FoV}_{Bi} \approx 2 a tan ( \frac{{W}_{Bi}}{2 DOE} ) = 2 a tan ( \frac{IPD}{2} ( \frac{1}{{D}_{Int}} - \frac{1}{DOE} ) ) .$$

In some embodiments, moving the projected scene closer to the eye, as the eye accommodate, results in the resolution decreasing. This might cause a problem with stereoscopic vision known as the Vergence-Accommodation Conflict. Relative to relaxed eye where the object is projected at infinity, the eye accommodation power as a function of virtual object distance is given by the reciprocal of the virtual object distance. To solve the Vergence-Accommodation Conflict, tunable lenses as described above may be used by directly applying negative of the accommodation power (added to any power the tunable lens has to account for) for a system designed to work with relaxed eyes. If the range of accommodation needed of the projected virtual object plane is small it can be handled by light field display.

For example, the image/object distance perceived by the eye (DOE) is related to the accommodation power (AP) of the eye via the following relationship:

$$D O E = \frac{1}{A P} .$$

With the above described systems and devices, ways to force the eye to accommodate to perceive a meaningful image may include:
1) Using external lens/tunable lens; or
2) Work within the correction range of the light field display to shift the correction power.

As noted in the above-mentioned reference, for un-aberrated eye, the intersection point on the retina of the incoming rays is only dependent on the angle of incidence at the pupil. Hence, in some embodiments, if the total system is reduced to a single lens and an un-aberrated eye, the light-field and image distance may be calculated more readily. Formulas for lenses addition are well known in the art. Using an external lens with accommodation power to give a perception of certain image distance, the net power (NP) can be calculated using the equivalent power of the external lens power (ELP) and the accommodation power of the eye, in addition to any spherical error (SE) of the eye:

$$N P = A P + S E + E L P - D E L ( A P + S E ) E L P$$

where DEL is the distance between the pupil/eye lens to the external lens.

If lightfield that corrects for power of P_{LF} (within the correction range of light field around the center of quality P_{Q}^{OS}), then NP has to be equal to this value to generate a meaningful image on retina:

$${P}_{L F} = A P + S E + E L P - D E L ( A P + S E ) E L P = \frac{1}{D O E} + S E + E L P - D E L ( A P + S E ) E L P .$$

Having this, the image distance/inverse distance can be calculated and passed to light field rendering algorithm based on the desired image distance. In some embodiments, in this is related to the P_{LF} as follows:

$$\frac{1}{D O {E}_{Algorithm}} = {P}_{L F} .$$

With the unified implementation the image/object is set at the real desired image distance then the phase element method is used to correct, using light field, for the power P_{LF} as calculated above.

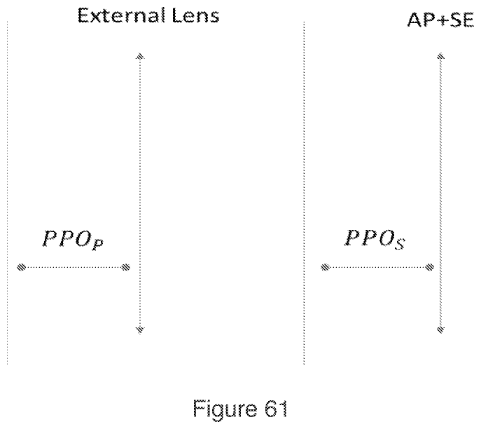

Also, as illustrated schematically in FIG. 61, in some embodiments, the primary (P subscript) and secondary (S subscript) principal planes offsets (PPO) of the external power and eye residual power (residual to the non-aberrated eye power that equals the reciprocal of the eye depth=AP+SE as noted in the equation above), consisting NP, can be calculated using:

$$P P {O}_{P} = \frac{E L P D E L}{N P} ; and \\ P P {O}_{S} = \frac{( A P + S E ) D E L}{N P} .$$

These values can be used to calculate the intersection point on the pupil along with the net power, where the distance between the principal planes is collapsed and the net power is modelled as a thin lens placed at the merged plane.

### Cases of Non-Axis Object/Viewer

In some embodiments, as discussed above, a light field raytracing algorithm or method may be designed to project light-field image through a source of aberration or optical element (i.e. eye lens or phase element) to the retina with the optical axis (z axis here) assumed to pass through the center of the display to the center of the optical element perpendicularly. For example, this is the case in the exemplary embodiments of methods 1100 and 1900. However, these methods may be expanded to consider cases where the pupil, aberration/optical element and display are not as described above.

For example, in some embodiments, the following steps may be followed:
1) The central position and directional vector of the optical element or source of aberration is noted. Given by V_{O}= [symbol] a_{O}^{x}, a_{O}^{y}, a_{O}^{z} [symbol] and P_{O}=(x_{O}, y_{O}, z_{O})
2) If the element directional vector is not aligned with the z axis (propagation axis) a_{O}^{x} or a_{O}^{y} does not equal zero. Do rotation transformation to align it to the optical axis.
3) If the optical element is not centered at the rotated optical axis (z axis in this case); translational transformation can be done to center pupil position at axis.
4) Use the rotated coordinates to calculate the virtual positions of the pixels and lenslets as explained. And either keep using it or rotate the coordinates back (using inverse rotation matrix for rotation) to use the lightfield rendering algorithm regularly. If the space is rotated back, then calculate the optical element characteristics such as surface normal and profile (PCT application PCT/IB2021/051868).

In some embodiments, rotations and translations may be handled using rotation/translation matrices for the reference coordinate system. However, in some cases, rotations may be implemented using quaternions or any known method in the art.

For example, in some embodiments, the rotation angles may be computed using:

$${γ}_{x} = \frac{abs ( {a}_{O}^{y} )}{{a}_{O}^{y}} a cos ( \frac{〈 {a}_{O}^{x} , {a}_{O}^{y} , {a}_{O}^{z} 〉 \cdot 〈 0 , 0 , 1 〉}{\vert 〈 0 , {a}_{O}^{y} , {a}_{O}^{z} 〉 \vert} ) ;$$

$${R}_{x} = [ \begin{matrix}1 & 0 & 0 \\ 0 & cos ( {γ}_{x} ) & - sin ( {γ}_{x} ) \\ 0 & sin ( {γ}_{x} ) & cos ( {γ}_{x} )\end{matrix} ] ;$$

$$〈 {a}_{Ox}^{x} , {a}_{Ox}^{y} , {a}_{Ox}^{z} 〉 = {R}_{x} 〈 {a}_{O}^{x} , {a}_{O}^{y} , {a}_{O}^{z} 〉 ;$$

$${γ}_{γ} = \frac{abs ( {a}_{Ox}^{x} )}{{a}_{Ox}^{x}} a cos ( \frac{〈 {a}_{Ox}^{x} , {a}_{Ox}^{y} , {a}_{Ox}^{z} 〉 \cdot 〈 0 , 0 , 1 〉}{\vert {R}_{x} 〈 {a}_{Ox}^{x} , 0 {a}_{Ox}^{z} 〉 \vert} ; and$$

$${R}_{y} = [ \begin{matrix}cos ( {γ}_{γ} ) & 0 & - sin ( {γ}_{γ} ) \\ 0 & 1 & 0 \\ sin ( {γ}_{γ} ) & 0 & cos ( {γ}_{γ} )\end{matrix} ] .$$

where in this example there is no need to transform around the z axis.

Then, the new rotated coordinates (rot superscripts) may be written as:

$$[ \begin{matrix}{x}^{rot} \\ {y}^{rot} \\ {z}^{rot}\end{matrix} ] = {R}_{y} {R}_{x} [ \begin{matrix}x \\ y \\ z\end{matrix} ]$$

which allows the calculation of the transformed coordinates of the pixel and LFSE centers using the rotation matrices. Then the translations (tr superscripts) may be computed as:

$${x}^{rot , tr} = x - {x}_{0}^{rot} ; and \\ {y}^{rot , tr} = y - {y}_{0}^{rot} .$$

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are hereby expressly incorporated by reference and are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

While the present disclosure describes various exemplary embodiments, the disclosure is not so limited. To the contrary, the disclosure is intended to cover various modifications and equivalent arrangements included within the general scope of the present disclosure.

1. A vision-based testing device for digitally implementing a vision-based test for a user using both their left and right eye simultaneously, the device comprising:
left and right display portions comprising respective pixel arrays;
corresponding light field shaping element (LFSE) arrays of light field shaping elements respectively disposed at a distance from said display portions so to at least partially govern respective left and right light fields projected on the user's left and right eye, respectively, wherein perception of said respective left and right light fields is at least partially constrained to the left and right eye, respectively; and
a digital data processor operable on pixel data for designated visual digital test content, to simultaneously render said designated visual digital test content via said respective pixel arrays in accordance with the vision-based test to be respectively projected toward respective user pupil locations in accordance with respective light field view zones generated via said respective pixel arrays and corresponding LFSE arrays to be simultaneously perceived by the left and right eye, respectively, to be at a common virtual position relative to the left and right eye so to invoke a natural binocular eye vergence response corresponding to said common virtual position.

2. The vision-based testing device of claim 1, wherein said common virtual position comprises a virtual depth position relative to said display portions.

3. The vision-based testing device of claim 1, wherein said left and right display portions comprise respective displays, and wherein said corresponding LFSE arrays comprise respective microlens arrays.

4. The vision-based testing device of claim 1, wherein said perception of said respective left and right light fields is at least partially constrained to the left and right eye via a physical barrier.

5. The vision-based testing device of claim 1, wherein said LFSE arrays comprise a microlens array.

6. The vision-based testing device of claim 1, wherein said common virtual position is a variable three-dimensional (3D) position that varies during execution of the vision-based test to dynamically adjust a perceived depth location of said designated visual digital test content and thereby invoke a variable binocular eye vergence response thereto.

7. The vision-based testing device of claim 6, wherein the vision-based test comprises a vergence test.

8. The vision-based testing device of claim 1, wherein said common virtual position is a variable two-dimensional (2D) location on a plane parallel to said display portions that varies during execution of the test to dynamically adjust a common perceived lateral location of said designated visual digital test content.

9. The vision-based testing device of claim 8, wherein the vision-based test comprises at least one of a saccades test or a smooth pursuit test.

10. The vision-based testing device of claim 1, wherein said designated visual digital test content comprises at least one of an optotype, a symbol, an image, a spot or a flash.

11. The vision-based testing device of claim 1, wherein said digital data processor is operable to adjust rendering of said designated visual digital test content via said corresponding LFSE arrays so to accommodate for a visual aberration in at least one of the left or right eye.

12. The vision-based testing device of claim 11, wherein said visual aberration comprises distinct respective visual aberrations for the left and right eye.

13. The vision-based testing device of claim 1, further comprising a pupil or eye tracking interface for tracking a motion of the left and right eye during execution of the vision-based test.

14. The vision-based testing device of claim 1, wherein said digital data processor is operable on said pixel data for each of the left and right display portions, respectively, to digitally:
project a given ray trace between each given pixel and a given pupil location given a direction of a light field emanated by said given pixel based on a given LFSE intersected thereby, to intersect said designated visual digital test content at said common virtual position or at its respective corresponding retinal image projections thereof; and
for each said given pixel, associate a given adjusted image pixel value designated as a function of said intersection.

15. The vision-based testing device of claim 6, further comprising a selectable or tunable lens to extend a dynamic range of said perceived depth location.

16. The vision-based testing device of claim 1, further comprising respective selectable or tunable lenses tunable to dynamically optically force the left and right eye to accommodate such that said designated visual digital test content is simultaneously perceived by the left and right eye, respectively, to be at said common virtual position relative.

17. The vision-based testing device of claim 1, wherein said digital data processor is further operable on pixel data for said designated visual digital test content to further adjust perception thereof in dynamically optically forcing the left and right eye to accommodate such that said designated visual digital test content is simultaneously perceived by the left and right eye, respectively, to be at said common virtual position.

18. The vision-based testing device of claim 1, wherein said digital data processor is further operable on pixel data for said designated visual digital test content to accommodate for a reduced user visual acuity such that said designated visual digital test content is simultaneously perceived by the left and right eye, respectively, to be at said common virtual position relative to the left and right eye without an intervening corrective lens adapted for said reduced visual acuity.

19. The vision-based testing device of claim 18, wherein said reduced user visual acuity comprises distinct respective reduced visual acuities for each of the right and left eye.
